(19) United States
(12) Patent Application Publication
(10) Pub. No.: US 2026/0298932 A1
(43) Pub. Date: Oct. 1, 2026

(54) GLYCOPROTEIN BIOMARKERS FOR OVARIAN CANCER DIAGNOSIS

(71) Applicant: PROSEEK BIO PTY LTD., Brisbane, Queensland (AU)

(72) Inventor: Michelle Mei Chih HILL, Brisbane, Queensland (AU)

(73) Assignee: PROSEEK BIO PTY LTD., Brisbane, Queensland (AU)

(21) Appl. No.: 19/480,758

(22) PCT Filed: May 3, 2024
(86) PCT No.: PCT/AU2024/050435

§ 371 Date: Oct. 31, 2025

(30) Foreign Application Priority Data
May 3, 2023 (AU) 2023901317

(51) Int. Cl. G01N 33/57545; G01N 33/575; G01N 33/68

(52) U.S. Cl. CPC G01N 33/57545; G01N 33/57585; G01N 33/6842; G01N 2333/4713; G01N 2333/4716; G01N 2333/4724; G01N 2333/4745; G01N 2333/8125; G01N 2333/918

(57) ABSTRACT

Disclosed herein are biomarkers for ovarian cancer and uses thereof, such as in methods for detecting the presence, and recurrence of ovarian cancer. Also disclosed are methods for treating and methods of monitoring the treatment and relapse of ovarian cancer, as well as to kits and compositions for use in such methods.

This application claims priority to Australian Provisional Patent Application No. 2023901317 entitled “Glycoprotein Biomarkers for Ovarian Cancer Diagnosis” filed 3 May 2023, the contents of which are incorporated herein by reference in their entirety.

### FIELD OF THE INVENTION

This invention relates generally to biomarkers for ovarian cancer and uses thereof, such as in methods for detecting the presence, and recurrence of ovarian cancer. The invention also relates to methods for treating and methods of monitoring the treatment and relapse of ovarian cancer, as well as to kits and compositions for use in such methods.

### BACKGROUND OF THE INVENTION

With non-specific symptoms and lack of screening program, most ovarian cancers are diagnosed at a late stage and with poor outcomes (see, Nash & Menon, 2020). The existing ovarian cancer tests, transvaginal ultrasound and serum CA125, are not specific to cancer, being unable to accurately distinguish benign mass from cancer. Indeed, two large ovarian cancer screening trials using a mixture of these modalities for screening found a lack of mortality benefit after long-term follow-up (see, Menon et al., 2021; Pinsky et al., 2016). Furthermore, randomized trials of ovarian cancer screening using existing tests have reported unnecessary surgery for women without cancer (see, Henderson, Webber, & Sawaya, 2018). The search for more specific ovarian cancer biomarkers has produced approved blood marker HE4 for monitoring ovarian cancer disease progression, as well as several multi-marker index assays for triaging (see, Muinao, Deka Boruah, & Pal, 2019; Trinidad, Tetlow, Bantis, & Godwin, 2020) but a highly specific and sensitive test for ovarian cancer screening remains elusive.

Cancer is associated with alterations in glycosylation machinery and glycan structures on circulating proteins (see, Pinho & Reis, 2015). In ovarian cancer, the expression of several glycosylation enzymes are reported to be altered, including GnT-III (see, Lu et al., 2016), MGAT3 (see, Kohler et al., 2016), B3GNT5 (see, Alam et al., 2017), St6Gal-I sialyltransferase (see, Schultz et al., 2016), as well as several members of GALNT family (see, Sheta et al., 2017). Serum glycomics and glycoproteomics profiling studies in ovarian cancer have reported differences in N-glycans (see, Biskup et al., 2013; Biskup, Braicu, Sehouli, Tauber, & Blanchard, 2014; Dedova, Braicu, Sehouli, & Blanchard, 2019; Mitra et al., 2013), glycopeptides (see, Hayashi et al., 2019; Miyamoto et al., 2018) and glycoproteins (see, Wu et al., 2012; Wu, Xie, Nie, Buckanovich, & Lubman, 2013). As serum contains high abundance proteins that may cause interference in proteomics workflows, some researchers used ascites (see, Biskup, Braicu, Sehouli, Tauber, & Blanchard, 2017; Miyamoto et al., 2016), ovarian cyst fluid (see, Kuzmanov et al., 2013) or mouse model of ovarian cancer for their glycomic or glycoproteomic discovery (see, Huttenhain et al., 2019).

While most existing cancer markers (including CA125 and HE4) are glycoproteins, current tests do not monitor the glycosylation structures. However, several studies have shown that monitoring specific glycoforms of cancer markers improves cancer-specificity. For ovarian cancer, it was shown that measuring particular glycosylated forms of CA125 using lectin immunoassay (see, Gidwani et al., 2016) or glycosylation-specific antibodies (see, Bayoumy et al., 2020) significantly improved discrimination between benign endometriosis and ovarian cancer. Therefore, the inventors reasoned that a multi-marker panel of glycospecies-targeted glycoprotein biomarkers may achieve the high specificity and sensitivity required for cancer screening. To facilitate glycoprotein biomarker discovery and development, the inventors previously established a lectin magnetic bead array (LeMBA)-coupled mass spectrometry platform and successfully applied it to oesophageal adenocarcinoma (see, Shah et al., 2015; Shah et al., 2018) as well as canine hemangiosarcoma (see, Oungsakul et al., 2021).

LeMBA features single-step glycoprotein isolation from serum (without depletion) in a 96-well plate format, followed by on-bead trypsin digest and direct loading of the released peptides to the mass spectrometer (see, Choi, Loo, Dennis, O'Leary, & Hill, 2011). This 96-well plate-based workflow enables high throughput mass spectrometry screening and validation using the same technology. This approach differs from previous studies, where discovery and validation generally used different technologies. For example, Wu et al. screened high-abundant protein-depleted serum samples using 16 lectins printed on a slide with fluorescence detection, identifying higher binding of ovarian cancer serum proteins to the lectins LCA, UEA-I and SNA, compared to benign (see, Wu et al., 2012). LCA and UEA-I bind fucose on glycoproteins, while SNA binds sialic acid on glycoproteins. Column chromatography was then used to discover the differential LCA, UEA-I or SNA binding proteins in 34 serum samples, followed by in-plate lectin-ELISA assay for validation in 85 serum samples (see, Wu et al., 2012; Wu et al., 2013).

Apart from consistent technical platform, the quality and subtype homogeneity of clinical samples can have a significant impact on the success of biomarker discovery and validation. Ovarian cancer is a heterogenous disease, with subtypes classified by histology. The majority of ovarian cancers are epithelial ovarian cancers, of which ~70% are aggressive high grade serous ovarian cancers (HGSOC) that has high mortality (see, Nash & Menon, 2020), while the remaining 30% have relatively indolent disease, including low grade serous, endometrioid, clear cell carcinomas, and mucinous carcinomas. As biomarkers may differ for ovarian cancer subtypes, there exists a need for products and processes to identify and validate serum glycoprotein biomarkers for HGSOC, the most common and aggressive epithelial ovarian cancer subtype.

It is an object of the invention to address and/or ameliorate at least one of the foregoing problems or at least provide the public with a useful choice.

### SUMMARY OF THE INVENTION

The present invention is predicated in part on the identification of glycospecies of serum glycoproteins that are reliable indicators of a subject having ovarian cancer, as compared to healthy subjects (i.e., subjects having a healthy condition) and/or subjects with a benign ovarian neoplasm. Thus, subjects with ovarian cancer have a different serum glycosylation “signature” or “profile” to healthy patients and to subjects with a benign ovarian neoplasm. Accordingly, as described herein, detecting the level of one or more of these different types of glycosylation in a biological sample (such as a blood, serum or plasma sample) from a subject can be used to determine the likelihood of the presence or absence of ovarian cancer in a subject. Monitoring the levels of one or more of the types of glycosylation identified herein can also be used to monitor ovarian cancer relapse after treatment. Accordingly, in some aspects, monitoring the levels of one or more types of glycosylation identified herein can also be used to monitor the efficacy of treatment of ovarian cancer.

The present invention thus represents a significant advance over current technologies for the management of ovarian cancer. In certain advantageous embodiments, it relies upon measuring the level of at least one glycospecies of a glycoprotein. The present invention also provides robust biomarkers for determining the likelihood of the presence or absence of ovarian cancer.

In one aspect, the invention provides a method of determining an indicator used in assessing a likelihood of the presence or absence of an ovarian cancer in a subject, the method comprising, consisting, or consisting essentially of:
(i) determining a biomarker value that is measured or derived for a glycospecies of at least one glycoprotein biomarker (e.g., at least 1, 2, 3, 4, 5, 6, 7, 8, or 10 glycoprotein biomarkers) in a sample obtained from the subject, wherein the at least one glycoprotein is selected from alpha-1-acid glycoprotein 1 (A1AG1), alpha-1-acid glycoprotein 2 (A1AG2), alpha-1-antitrypsin (A1AT), alpha-1-antichymotrypsin (AACT), alpha-1-microglobulin (AMBP), alpha-2-antiplasmin (A2AP), alpha-2-HS-glycoprotein (FETUA), alpha-2-macroglobulin (A2MG), angiotensinogen (ANGT), carboxypeptidase N subunit 2 (CPN2), cholinesterase (CHLE), coagulation factor X (FA10), coagulation factor XII (FA12), complement C1q subcomponent subunit C (C1QC), complement component 2 (CO2), complement component 6 (CO6), complement component C7 (CO7), complement component C9 (CO9), complement factor B (CFAB), corticosteroid-binding globulin (CBG), fibrinogen alpha (FIBA), fibrinogen beta (FIBB), galectin-3-binding protein (LG3BP), gelsolin (GELS), haptoglobin (HPT), haptoglobin-related protein (HPTR), hemopexin (HEMO), heparin cofactor 2 (HEP2), inter-alpha-trypsin inhibitor heavy chain 1 protein (ITIH1), inter-alpha-trypsin inhibitor heavy chain 2 protein (ITIH2), inter-alpha-trypsin inhibitor heavy chain 3 protein (ITIH3), inter-alpha-trypsin inhibitor heavy chain 4 protein (ITIH4), insulin-like growth factor-binding protein 3 (IBP3), insulin-like growth factor-binding protein complex acid labile subunit (ALS), kininogen-1 (KNG1), lumican (LUM), N-acetylmuramoyl-L-alanine amidase (PGRP2), plasma protease C1 inhibitor (IC1), plasminogen (PLMN), prothrombin (THRB), serotransferrin (TRFE), and serum paraoxonase/arylesterase 1 (PON1); and
(ii) determining the indicator using the biomarker value(s), wherein the indicator is at least partially indicative of the likelihood of the presence or absence of the ovarian cancer in the subject.

In some embodiments, the at least one glycoprotein is selected from alpha-1-antitrypsin (A1AT), alpha-1-antichymotrypsin (AACT), complement component C9 (CO9), haptoglobin (HPT), Inter-alpha-trypsin inhibitor heavy chain H3 (ITIH3), alpha-2-macroglobulin (A2MG), insulin-like growth factor-binding protein complex acid labile subunit (ALS), insulin-like growth factor binding protein 3 (IBP3), and paraoxonase/arylesterase 1 (PON1).

In another aspect, the invention provides a method of determining an indicator used in assessing the likelihood of an early onset of ovarian cancer being present in a subject, the method comprising, consisting, or consisting essentially of:
(i) determining a biomarker value that is measured or derived for a glycospecies of one or more glycoprotein biomarkers (e.g., at least 1, 2, 3, 4, 5, 6, 7, 8, or 10 glycoprotein biomarkers) in a sample obtained from the subject, wherein the one or more glycoprotein is selected from alpha-1-acid glycoprotein 1 (A1AG1), alpha-1-acid glycoprotein 2 (A1AG2), alpha-1-antitrypsin (A1AT), alpha-1-antichymotrypsin (AACT), alpha-1-microglobulin (AMBP), alpha-2-antiplasmin (A2AP), alpha-2-HS-glycoprotein (FETUA), alpha-2-macroglobulin (A2MG), angiotensinogen (ANGT), carboxypeptidase N subunit 2 (CPN2), cholinesterase (CHLE), coagulation factor X (FA10), coagulation factor XII (FA12), complement C1q subcomponent subunit C (C1QC), complement component 2 (CO2), complement component C9 (CO9), complement factor B (CFAB), corticosteroid-binding globulin (CBG), fibrinogen alpha (FIBA), fibrinogen beta (FIBB), galectin-3-binding protein (LG3BP), gelsolin (GELS), haptoglobin (HPT), haptoglobin-related protein (HPTR), hemopexin (HEMO), heparin cofactor 2 (HEP2), inter-alpha-trypsin inhibitor heavy chain 1 protein (ITIH1), inter-alpha-trypsin inhibitor heavy chain 2 protein (ITIH2), inter-alpha-trypsin inhibitor heavy chain 3 protein (ITIH3), inter-alpha-trypsin inhibitor heavy chain 4 protein (ITIH4), insulin-like growth factor-binding protein 3 (IBP3), insulin-like growth factor-binding protein complex acid labile subunit (ALS), kininogen-1 (KNG1), lumican (LUM), N-acetylmuramoyl-L-alanine amidase (PGRP2), plasma protease C1 inhibitor (IC1), plasminogen (PLMN), prothrombin (THRB), serotransferrin (TRFE), and serum paraoxonase/arylesterase 1 (PON1); and
(ii) determining the indicator using the biomarker value(s), wherein the indicator is at least partially indicative of the likelihood of the of an early onset of ovarian cancer being present in the subject.

In some embodiments, the subject is asymptomatic.

In some embodiments, the indicator is determined by comparing a biomarker value in a first sample to a respective biomarker value in a second sample, wherein the second sample was taken at a later time than the first sample.

In a further aspect, the invention provides method for treating an ovarian cancer in a subject, the method comprising, consisting, or consisting essentially of:
(i) determining a biomarker value that is measured or derived for a glycospecies of one or more glycoprotein biomarkers (e.g., at least 1, 2, 3, 4, 5, 6, 7, 8, or 10 glycoprotein biomarkers) in a sample obtained from the subject, wherein the one or more glycoprotein is selected from alpha-1-acid glycoprotein 1 (A1AG1), alpha-1-acid glycoprotein 2 (A1AG2), alpha-1-antitrypsin (A1AT), alpha-1-antichymotrypsin (AACT), alpha-1-microglobulin (AMBP), alpha-2-antiplasmin (A2AP), alpha-2-HS-glycoprotein (FETUA), alpha-2-macroglobulin (A2MG), angiotensinogen (ANGT), carboxypeptidase N subunit 2 (CPN2), cholinesterase (CHLE), coagulation factor X (FA10), coagulation factor XII (FA12), complement C1q subcomponent subunit C (C1QC), complement component 2 (CO2), complement component C9 (CO9), complement factor B (CFAB), corticosteroid-binding globulin (CBG), fibrinogen alpha (FIBA), fibrinogen beta (FIBB), galectin-3-binding protein (LG3BP), gelsolin (GELS), haptoglobin (HPT), haptoglobin-related protein (HPTR), hemopexin (HEMO), heparin cofactor 2 (HEP2), inter-alpha-trypsin inhibitor heavy chain 1 protein (ITIH1), inter-alpha-trypsin inhibitor heavy chain 2 protein (ITIH2), inter-alpha-trypsin inhibitor heavy chain 3 protein (ITIH3), inter-alpha-trypsin inhibitor heavy chain 4 protein (ITIH4), insulin-like growth factor-binding protein 3 (IBP3), insulin-like growth factor-binding protein complex acid labile subunit (ALS), kininogen-1 (KNG1), lumican (LUM), N-acetylmuramoyl-L-alanine amidase (PGRP2), plasma protease C1 inhibitor (IC1), plasminogen (PLMN), prothrombin (THRB), serotransferrin (TRFE), and serum paraoxonase/arylesterase 1 (PON1);
(ii) determining the indicator using the biomarker value(s); and
(iii) administering an effective amount of an anti-cancer treatment to the subject on the basis that the indicator is at least partially indicative of the likelihood of the presence of ovarian cancer in the subject.

In some embodiments, the anti-cancer treatment is chemotherapy, radiotherapy, and/or immunotherapy. By way of an example, the anti-cancer treatment may be an immunotherapy that targets an immune checkpoint molecule (e.g., PD1, PD-L1, CTLA4, and the like).

Typically, the biomarker value is at least partially indicative of a concentration of the glycospecies of one or more glycoproteins in the sample obtained from the subject. In some embodiments, the biomarker value includes the abundance of the glycospecies of one or more glycoproteins.

In some embodiments, the level of the glycospecies of a glycoprotein is reduced relative to the level of the biomarker that correlates with a healthy subject, and the indicator is thereby determined to be at least partially indicative of the subject having ovarian cancer.

In some embodiments, the level of the glycospecies of a glycoprotein is about the same as the level of the biomarker that correlates with a healthy subject, and the indicator is determined to be at least partially indicative of the absence of ovarian cancer in the subject.

In some embodiments, the indicator comprises a biomarker value for at least 2, 3, 4, 5, 6, 7, 8, 9, 10, 15, 20 (and every integer in between) glycoprotein biomarkers.

In some embodiments, the biomarker value comprises a ratio of the level of the glycospecies of a glycoprotein to the total level of the glycoprotein in the sample.

In some embodiments, a first biomarker value is measured or derived for a first glycospecies of a glycoprotein and a second biomarker value is measured or derived for a second glycospecies of the glycoprotein, wherein the first glycospecies of the glycospecies is differently expressed between a sample from a subject with ovarian cancer and a sample from a healthy subject, and the second glycospecies of the glycoprotein is not so differentially expressed.

Typically, the level of an individual glycospecies of a glycoprotein is determined by contacting the sample with a glycan-binding molecule specific for the glycospecies of the glycoprotein, under conditions that permit binding of the glycan-binding molecule to the glycospecies of the glycoprotein. The glycan-binding molecule is generally selected from the group consisting of a lectin, a glycospecific antibody, a glycospecific aptamer, a glycospecific peptide, and a glycospecific small molecule.

In some embodiments, wherein the lectin is selected from Aleuria aurantia lectin (AAL), Solanum tuberosum lectin (STL), and Sambucus nigra agglutinin (SNA).

In some embodiments, the individual glycospecies (i.e., defined by the glycan-binding molecule (e.g., lectin) and the glycoprotein to which it binds) that are differentially expressed between a sample from a subject with ovarian cancer and a sample from a healthy subject are selected from Table 1:

TABLE 1

| | Glycoprotein | |
|---|---|---|
| Lectin | Overexpressed | Underexpressed |
| AAL | A1AG1, A1AG2, A1AT, AACT, | A2MG, AMBP, C1QC, CBG, CHLE, |
| | CFAB, CO2, CO9, A10, FIBA, | FETUA, GELS, IBP3, ITIH1, ITIH2, |
| | FIBB, HPT, HPTR, ITIH3, | PON1, TRFE |
| | ITIH4 | |
| SNA | A1AG1, A1AT, AACT, CFAB, | A1AG2, A2AP, A2MG, ALS, AMBP, |
| | CO9, FA10, FIBA, FIBB, HPT, | ANGT, C1QC, CBG, CHLE, CPN2, |
| | HPTR, IC1, ITIH3 | FA10, FA12, FETUA, GELS, |
| | | HEMO, HEP2, IBP3, ITIH1, ITIH2, |
| | | KNG1, PLMN, PON1, PGRP2, |
| | | THRB, TRFE |
| STL | CO9, FIBA, FIBB, HPT, HPTR, | A2MG, A2AP, ALS, AMBP, ANGT, |
| | ITIH3, LUM | C1QC, CBG, CHLE, CPN2, FA10, |
| | | FA12, FETUA, GELS, HEP2, |
| | | HPTR, IBP3, ITIH1, ITIH2, ITIH4, |
| | | LG3BP, KNG1, PGRP2, PLMN, |
| | | PON1, TRFE |

In some embodiments, the method comprises determining two or more biomarker values that are measured or derived from two or more glycospecies of the same glycoprotein. Alternatively, the method may comprise determining three or more biomarker values that are measured or derived from three or more glycospecies of the same glycoprotein. Alternatively, the method may comprise determining four or more biomarker values that are measured or derived from four or more glycospecies of the same glycoprotein. Alternatively, the method may comprise determining five or more biomarker values that are measured or derived from five or more glycospecies of the same glycoprotein.

In some embodiments, the ovarian cancer is high grade serous ovarian cancer (HGSOC).

In some embodiments, upon determining that a subject is likely to have an ovarian cancer, the methods further comprise exposing the subject to a diagnosis and treatment regimen for treating the ovarian cancer. For example, the diagnosis and treatment regimen may comprise tissue sampling for pathology, surgery, radiotherapy, chemotherapy, or an immunotherapy. In some embodiments, the surgery removes all or part of the ovaries.

In some embodiments, the determination methods described above and/or elsewhere herein, are performed by a person who exposes the subject to the treatment regimen. In some alternative embodiments, the sample from the subject is provided to another person (e.g., a person in a laboratory) who performs the determination method and provides the results of the determination method to the person who exposes the subject to the treatment regimen.

In another aspect, the present invention provides a composition for determining an indicator used in assessing a likelihood that a subject has ovarian cancer, the composition comprising, consisting, or consisting essentially of at least one glycospecies of a glycoprotein, and at least one glycan-binding molecule specific for the glycospecies of the glycoprotein, wherein the glycoprotein is selected from alpha-1-acid glycoprotein 1 (A1AG1), alpha-1-acid glycoprotein 2 (A1AG2), alpha-1-antitrypsin (A1AT), alpha-1-antichymotrypsin (AACT), alpha-1-microglobulin (AMBP), alpha-2-antiplasmin (A2AP), alpha-2-HS-glycoprotein (FETUA), alpha-2-macroglobulin (A2MG), angiotensinogen (ANGT), carboxypeptidase N subunit 2 (CPN2), cholinesterase (CHLE), coagulation factor X (FA10), coagulation factor XII (FA12), complement C1q subcomponent subunit C (C1QC), complement component 2 (CO2), complement component C9 (CO9), complement factor B (CFAB), corticosteroid-binding globulin (CBG), fibrinogen alpha (FIBA), fibrinogen beta (FIBB), galectin-3-binding protein (LG3BP), gelsolin (GELS), haptoglobin (HPT), haptoglobin-related protein (HPTR), hemopexin (HEMO), heparin cofactor 2 (HEP2), inter-alpha-trypsin inhibitor heavy chain 1 protein (ITIH1), inter-alpha-trypsin inhibitor heavy chain 2 protein (ITIH2), inter-alpha-trypsin inhibitor heavy chain 3 protein (ITIH3), inter-alpha-trypsin inhibitor heavy chain 4 protein (ITIH4), insulin-like growth factor-binding protein 3 (IBP3), insulin-like growth factor-binding protein complex acid labile subunit (ALS), kininogen-1 (KNG1), lumican (LUM), N-acetylmuramoyl-L-alanine amidase (PGRP2), plasma protease C1 inhibitor (IC1), plasminogen (PLMN), prothrombin (THRB), serotransferrin (TRFE), and serum paraoxonase/arylesterase 1 (PON1).

In some preferred embodiments, the glycoprotein is selected from alpha-1-antitrypsin (A1AT), alpha-1-antichymotrypsin (AACT), complement component C9 (CO9), haptoglobin (HPT), Inter-alpha-trypsin inhibitor heavy chain H3 (ITIH3), alpha-2-macroglobulin (A2MG), insulin-like growth factor-binding protein complex acid labile subunit (ALS), insulin-like growth factor binding protein 3 (IBP3), and paraoxonase/arylesterase 1 (PON1).

In some embodiments, the composition comprises two or more glycospecies of a glycoprotein.

In yet another aspect, the present invention provides a complex comprising, consisting, or consisting essentially of at least one glycospecies of a glycoprotein a glycan-binding molecule that is specific for the glycospecies of the glycoprotein, wherein the glycoprotein is selected from alpha-1-acid glycoprotein 1 (A1AG1), alpha-1-acid glycoprotein 2 (A1AG2), alpha-1-antitrypsin (A1AT), alpha-1-antichymotrypsin (AACT), alpha-1-microglobulin (AMBP), alpha-2-antiplasmin (A2AP), alpha-2-HS-glycoprotein (FETUA), alpha-2-macroglobulin (A2MG), angiotensinogen (ANGT), carboxypeptidase N subunit 2 (CPN2), cholinesterase (CHLE), coagulation factor X (FA10), coagulation factor XII (FA12), complement Clq subcomponent subunit C (C1QC), complement component 2 (CO2), complement component C9 (CO9), complement factor B (CFAB), corticosteroid-binding globulin (CBG), fibrinogen alpha (FIBA), fibrinogen beta (FIBB), galectin-3-binding protein (LG3BP), gelsolin (GELS), haptoglobin (HPT), haptoglobin-related protein (HPTR), hemopexin (HEMO), heparin cofactor 2 (HEP2), inter-alpha-trypsin inhibitor heavy chain 1 protein (ITIH1), inter-alpha-trypsin inhibitor heavy chain 2 protein (ITIH2), inter-alpha-trypsin inhibitor heavy chain 3 protein (ITIH3), inter-alpha-trypsin inhibitor heavy chain 4 protein (ITIH4), insulin-like growth factor-binding protein 3 (IBP3), insulin-like growth factor-binding protein complex acid labile subunit (ALS), kininogen-1 (KNG1), lumican (LUM), N-acetylmuramoyl-L-alanine amidase (PGRP2), plasma protease C1 inhibitor (IC1), plasminogen (PLMN), prothrombin (THRB), serotransferrin (TRFE), and serum paraoxonase/arylesterase 1 (PON1).

In some preferred embodiments, the glycoprotein is selected from alpha-1-antitrypsin (A1AT), alpha-1-antichymotrypsin (AACT), complement component C9 (CO9), haptoglobin (HPT), inter-alpha-trypsin inhibitor heavy chain H3 (ITIH3), alpha-2-macroglobulin (A2MG), insulin-like growth factor-binding protein complex acid labile subunit (ALS), insulin-like growth factor binding protein 3 (IBP3), and paraoxonase/arylesterase 1 (PON1).

In some embodiments, the glycan-binding molecule is selected from the group consisting of a lectin, a glycospecific antibody, a glycospecific aptamer, a glycospecific peptide, and a glycospecific small molecule.

In some embodiments, the lectin is selected from Aleuria aurantia lectin (AAL), Erythrina cristagalli agglutinin (ECA), floribunda agglutinin (WFA), Solanum tuberosum lectin (STL), concanavalin A (ConA), Sambucus nigra agglutinin (SNA), and Phaseolus vulgaris Leucoagglutinin (L-PHA).

In some embodiments, the lectin is selected from AAL, SNA and STL.

In some embodiments, the individual glycospecies (i.e., defined by the glycan-binding molecule (e.g., lectin) and the glycoprotein to which it binds) are selected from Table 1.

In another aspect, the present invention provides a composition comprising a sample obtained from a subject, and at least one reagent for measuring a glycospecies.
In still yet another aspect, the invention provides a method of determining an indicator used in distinguishing between an ovarian cancer in a subject and benign ovarian biomass in a subject, the method comprising, consisting, or consisting essentially of:
(i) determining a biomarker value that is measured or derived for a glycospecies of at least one glycoprotein biomarker (e.g., at least 1, 2, 3, 4, 5, 6, 7, 8, or 10 glycoprotein biomarkers) in a sample obtained from the subject, wherein the at least one glycoprotein is selected from alpha-1-acid glycoprotein 1 (A1AG1), alpha-1-acid glycoprotein 2 (A1AG2), alpha-1-antitrypsin (A1AT), alpha-1-B glycoprotein (A1BG), alpha-2-antiplasmin (A2AP), alpha-2-macroglobulin (A2MG), alpha-1-antichymotrypsin (AACT), insulin-like growth factor-binding protein complex acid labile subunit (ALS), alpha-1-microglobulin (AMBP), angiotensinogen (ANGT), apolipoprotein B (APOB), ceruloplasmin (CERU), complement C1q subcomponent subunit C (C1QC), corticosteroid-binding globulin (CBG), cholinesterase (CHLE), complement factor B (CFAB), complement factor I (CFAI), complement component 2 (CO2), complement component 6 (CO6), complement component 7 (CO7), complement component 9 (CO9), carboxypeptidase N subunit 2 (CPN2), coagulation factor V (FA5), coagulation factor X (FA10), coagulation factor XII (FA12), alpha-2-HS-glycoprotein (FETUA), fibrinogen alpha (FIBA), fibrinogen beta (FIBB), gelsolin (GELS), hemopexin (HEMO), heparin cofactor 2 (HEP2), haptoglobin (HPT), haptoglobin-related protein (HPTR), insulin-like growth factor-binding protein 3 (IBP3), plasma protease C1 inhibitor (IC1), inter-alpha-trypsin inhibitor heavy chain 1 protein (ITIH1), inter-alpha-trypsin inhibitor heavy chain 2 protein (ITIH2), inter-alpha-trypsin inhibitor heavy chain 3 protein (ITIH3), inter-alpha-trypsin inhibitor heavy chain 4 protein (ITIH4), kininogen-1 (KNG1), galectin-3 binding protein (LG3BP), pigment epithelium-derived factor (PEDF), plasminogen (PLMN), paraoxonase/arylesterase 1 (PON1), N-acetylmuramoyl-L-alanine amidase (PGRP2), prothrombin (THRB), serotransferrin (TRFE), and vitamin D-binding protein (VTDB); and
(ii) determining the indicator using the biomarker value(s), wherein the indicator is at least partially indicative of the likelihood of a subject having an ovarian cancer or a benign ovarian biomass.

Typically, the level of an individual glycospecies of a glycoprotein is determined by contacting the sample with a glycan-binding molecule specific for the glycospecies of the glycoprotein, under conditions that permit binding of the glycan-binding molecule to the glycospecies of the glycoprotein. The glycan-binding molecule is generally selected from the group consisting of a lectin, a glycospecific antibody, a glycospecific aptamer, a glycospecific peptide, and a glycospecific small molecule.

In some embodiments, wherein the lectin is selected from Aleuria aurantia lectin (AAL), Solanum tuberosum lectin (STL), and Sambucus nigra agglutinin (SNA).

In some embodiments, the individual glycospecies (i.e., defined by the glycan-binding molecule (e.g., lectin) and the glycoprotein to which it binds) that are differentially expressed between a sample from a subject with ovarian cancer and a sample from a subject with a benign ovarian biomass are selected from Table 2:

TABLE 2

| | Glycoprotein | |
|---|---|---|
| Lectin | Overexpressed | Underexpressed |
| AAL | A1AG1, A1AG2, A1AT, A1BG, | A2MG, GELS, IBP3, ITIH2 |
| | AACT, CERU, CFAB, CFAI, | |
| | CO2, CO9, FA5, FA10, FIBA, | |
| | FIBB, HPT, HPTR, IC1, ITIH3, | |
| | ITIH4, PEDF, VTDB | |
| SNA | A1AT, AACT, CO9, FA10, | A1AG2, A2AP, A2MG, ALS, AMBP, |
| | FIBA, FIBB, HPT, HPTR, | ANGT, APOB, C1QC, CBG, CHLE, |
| | ITIH3 | CO6, CO7, CPN2, FA10, FA12, |
| | | FETUA, GELS, IBP3, ITIH1, ITIH2, |
| | | KNG1, PLMN, PON1, PGRP2, |
| | | THRB, TRFE |
| STL | CO9, FIBB, HPT, HPTR | A2MG, A2AP, ALS, AMBP, ANGT, |
| | | CBG, CHLE, CPN2, FA10, FETUA, |
| | | GELS, HPTR, IBP3, ITIH1, ITIH2, |
| | | KNG1, PLMN, PON1, TRFE |

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1 provides a schematic of the biomarker study design. Discovery and validation of high grade serous ovarian cancer (HGSOC) biomarkers was conducted in two phases, starting from separate clinical cohorts (1) and sera collection (2). Lectin selection (3) was based on literature for discovery phase and discovery results for validation phase. Both phases use LeMBA (4), liquid handler-assisted pulldown (5) and on-bead digestion (6). Shotgun mass spectrometry was conducted for discovery phase (7) followed by discovery of candidates (8) for development of a targeted mass spectrometry assay (9) for validation phase. Both univariate and multivariate analyses were conducted for biomarker validation (10).

FIG. 2 illustrates the biomarker discovery. Volcano and two-way scatter plots visualising the differentially abundant proteins and correlated proteins respectively, between the benign and HGSOC (A, C, E) and healthy and HGSOC (B, D, F) clinical comparisons for UKOPS and UKCTOCS sample sets. The volcano plots highlight all differential glycoproteins according to the criteria p<0.05, Log₂ Fold Change >1. The scatter plots highlight select glycoproteins (Log₂ Fold Change >0.5) that are upregulated (green dots) and downregulated (red dots) in both sample sets. All candidates are labelled by the nomenclature “lectin-UniProt ID”.

FIG. 3 shows biomarker validation data. LeMBA-MRM data were analysed for peptides that are differentially abundant between benign and HGSOC (A, C, E, G), and healthy and HGSOC (B, D, F, H) for AAL lectin (A, B,), SNA lectin (C, D), STL lectin (E, F). Each dot in the volcano plot indicates a peptide, labelled only by the corresponding UniProt ID for the protein for visualization. The overlap between candidates for each lectin is shown in G, and H.

FIG. 4 illustrates the protein-protein interaction network for selected ovarian cancer protein biomarkers. STRING database was used to develop protein-protein interaction network for (A) the nine validated glycoprotein biomarkers from univariate analysis, (B) the six biomarkers from the multivariate signature and (C) the three predictive biomarkers. Nodes are the glycoprotein biomarkers labelled by GeneName. Edges denote interactions between proteins

FIG. 5 exemplifies the potential predictive biomarkers for ovarian cancer screening. Comparison of glycoprotein biomarker data between preclinical UKCTOCS (left panel, protein-level data) discovery case control set, and (right panel, showing all peptides measured) STL-pulldown validation case control for (A) CO9, (B) ITIH3, and (C) A2MG. For comparison, the unadjusted p-values are shown for both data sets. The UKCTOCS serum samples were collected 11.1±5.1 months prior to HGSOC diagnosis in a longitudinal study.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

It will be appreciated that the indefinite articles “a” and “an” are not to be read as singular indefinite articles or as otherwise excluding more than one or more than a single subject to which the indefinite article refers. For example, “a glycospecies” means one glycospecies or more than one glycospecies.

As used herein the terms “abundance,” “level,” and “amount” are used interchangeably herein to refer to a quantitative amount (e.g., weight or moles), a semi-quantitative amount, a relative amount (e.g., weight % or mole % within class), a concentration, and the like. Thus, these terms encompass absolute or relative amounts or concentrations of biomarkers in a sample.

As used herein, “and/or” refers to and encompasses any and all possible combinations of one or more of the associated listed items, as well as the lack of combinations when interpreted in the alternative (or).

The term “biomarker” broadly refers to any detectable compound, such as a protein, a peptide, a proteoglycan, a glycoprotein (e.g., a glycospecies of a glycoprotein), a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a sample. “Derived from” as used in this context refers to a compound that, when detected, is indicative of a particular molecule being present in the sample. For example, detection of a particular cDNA can be indicative of the presence of a particular RNA transcript in the sample. As another example, detection of or binding to a particular lectin can be indicative of the presence of a particular glycospecies of a glycoprotein in the sample. Here, a discriminating molecule or fragment is a molecule or fragment that, when detected, indicates presence or abundance of an above-identified compound. A biomarker can, for example, be isolated from a sample, directly measured in a sample, or detected in or determined to be in a sample. A biomarker can, for example, be functional, partially functional, or non-functional. In specific embodiments, the “biomarkers” include specific “glycospecies of a glycoprotein”, which are described in more detail below.

The term “biomarker value” refers to a value measured or derived for at least one corresponding biomarker of a subject and which is typically at least partially indicative of an abundance or concentration of a biomarker in a sample taken from the subject. Thus, the biomarker values could be measured biomarker values, which are values of biomarkers measured for the subject, or alternatively could be derived biomarker values, which are values that have been derived from one or more measured biomarker values, for example by applying a function to the one or more measured biomarker values. Biomarker values can be of any appropriate form depending on the manner in which the values are determined. For example, the biomarker values could be determined using high-throughput technologies such as sequencing platforms, array and hybridization platforms, mass spectrometry, immunoassays, immunofluorescence, flow cytometry, or any combination of such technologies. In one preferred example, the biomarker values relate to a level of abundance of a glycoprotein quantified using a technique such as mass spectrometry (e.g., LeMBA-coupled tandem mass spectrometry), lectin-immunoassay, or the like. In this case, the biomarker values can be in the form of peak intensity or area under the peak, which are a representation of the concentration of the biomarker within a sample, as will be appreciated by persons skilled in the art and as will be described in more detail below. In other preferred examples, the biomarker values are quantified using microfluidic-assisted lectin-capture immunoassays with fluorescence or spectroscopy-based detection.

The term “biomarker profile” refers to one or a plurality of one or more types of biomarkers (e.g., a glycospecies of a glycoprotein, etc.), or an indication thereof, together with a feature, such as a measurable aspect (e.g., biomarker value) of the biomarker(s). A biomarker profile may comprise a single biomarker whose level, abundance or amount correlates with a condition or clinical state (e.g., presence of ovarian cancer). Alternatively, a biomarker profile may comprise at least two such biomarkers or indications thereof, where the biomarkers can be in the same or different classes, such as, for example, a glycospecies of a glycoprotein and a nucleic acid. Thus, a biomarker profile may comprise at least 2, 3, 4, 5, 6, 7, 8, 9, 10, 15, 20, 25, 30, 35, 40, 45, 50, 55, 60, 65, 70, 75, 80, 85, 90, 95, or 100 or more biomarkers or indications thereof. A biomarker profile can further comprise one or more controls or internal standards. In certain embodiments, the biomarker profile comprises at least one biomarker, or indication thereof, that serves as an internal standard. In other embodiments, a biomarker profile comprises an indication of one or more types of biomarkers. The term “indication” as used herein in this context merely refers to a situation where the biomarker profile contains symbols, data, abbreviations or other similar indicia for a biomarker, rather than the biomarker molecular entity itself. The term “biomarker profile” is also used herein to refer to a biomarker value or combination of at least two biomarker values, wherein individual biomarker values correspond to values of biomarkers that can be measured or derived from one or more subjects, which combination is characteristic of a condition or clinical state or a prognosis for a condition or clinical state (e.g., presence of ovarian cancer). The term “profile biomarkers” is used to refer to a subset of the biomarkers that have been identified for use in a biomarker profile that can be used in performing a clinical assessment, such as to rule in or rule out a specific conditions or clinical states. The number of profile biomarkers will vary, but is typically of the order of 10 or less. In one example, a biomarker profile includes a profile of biomarkers selected from a glycospecies of A1AT, a glycospecies of AACT, a glycospecies of CO9, a glycospecies of HPT, a glycospecies of ITIH3, a glycospecies of A2MG, a glycospecies of ALS, a glycospecies of IBP3, and a glycospecies of PON1.

Throughout this specification and the claims which follow, unless the context requires otherwise, the work “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The term “control subject”, as used in the context of the present invention, may refer to a subject known to be affected with a disease conditions (e.g., ovarian cancer) (positive control), or to a subject known to be not affected or diagnosed with the disease condition (e.g., benign ovarian neoplasm or healthy subject) (negative control). It should be noted that a control subject that is known to be healthy, i.e., not suffering from the disease condition, may possibly suffer from another disease not tested/known. It is also understood that control subjects and healthy controls include data obtained and used as a standard, i.e., it can be used over and over again for multiple different subjects. In other words, for example, when comparing a subject sample to a control sample, the data from the control sample could have been obtained in a different set of experiments, for example, it could be an average obtained from a number of healthy subjects and not actually obtained at the time the data for the subject was obtained.

The term “correlating” generally refers to determining a relationship between one type of data with another or with a state. In various embodiments, correlating a glycospecies profile with the presence or absence of a conditions (e.g., a condition selected from a healthy control, ovarian cancer, or benign ovarian neoplasm) comprises determining the presence, absence or amount of at least one glycospecies in a subject that suffers from that condition, or in persons known to be free of that condition. In specific embodiments, a profile of glycospecies levels, absences or presences is correlated to a global probability or a particular outcome, using receiver operating characteristic (ROC) curves.

By “corresponding glycoprotein” is meant a glycoprotein biomarker that is structurally and/or functionally similar to or the same as a glycospecies biomarker. Representative corresponding glycoproteins include other glycospecies of the glycoprotein, and isoforms of the glycoprotein without any glycosylation.

As used herein, the terms “diagnosis”, “diagnosing”, “screening” and the like are used interchangeably herein to encompass determining the likelihood that a subject will develop or has a condition or clinical state (e.g., responsiveness or non-responsiveness to cancer therapy). These terms also encompass, for example, determining the level of clinical state (e.g., the presence of ovarian cancer), as well as in the context of rational therapy, in which the diagnosis guides therapy, including initial selection of therapy, modification of therapy (e.g., adjustment of treatment regimen), and the like. By “likelihood” is meant a measure of whether a subject with particular measured or derived biomarker values actually has a condition or clinical state (or not) based on a given mathematical model. An increased likelihood for example may be relative or absolute and may be expressed qualitatively or quantitatively. For instance, an increased likelihood may be determined simply by determining the subject's measured or derived biomarker values for one or more cancer therapy biomarkers and placing the subject in an “increased likelihood” category, based upon previous population studies. The term “likelihood” is also used interchangeably herein with the term “probability”.

The term “differential expression” of glycospecies as used herein, means qualitative and/or quantitative differences in the temporal and/or local glycospecies expression patterns, e.g., between a biological sample take from subjects with a condition as compared to a comparable sample taken from subjects lacking the condition. Thus, a differentially expressed glycospecies may qualitatively have its expression altered, including an activation or inactivation in, for example, a biological sample from a subject with a disease condition (e.g., ovarian cancer) or differential diagnosis condition (e.g., benign ovarian neoplasm) versus a healthy subject. The difference in glycospecies abundance may also be quantitative, e.g., in that abundance is modulated, i.e., either overexpressed, resulting in an increased amount of glycospecies, or underexpressed, resulting in a decreased amount of glycospecies. The degree to which glycospecies abundance differs need only be large enough to be quantified via standard quantification or characterisation techniques. For example, a glycospecies is differentially present between the samples if the amount of the glycospecies in one sample is significantly different (i.e., p<0.05) from the amount of the glycospecies in the other sample. It should be noted that if the glycospecies is detectable in one sample and not detectable in the other, then the glycospecies can be considered to be differentially present.

As used herein, a “glycoprotein” refers to a protein having oligosaccharides structures covalently attached to its amino acid side-chains. Glycoproteins can be associated with one or more types of glycosylation at a single or different sites. Glycoproteins that differ with respect to type of glycosylation generally have the same amino acid sequence or essentially the same amino acid sequence (e.g., isoforms, allelic variants and other variants are considered to have essentially the same amino acid sequence), while the glycan structures associated with a particular type of glycosylation differ by at least one glycan or linkage.

Most naturally occurring secreted proteins (or peptides) comprise carbohydrate or oligosaccharide moieties attached to the peptide via specific linkages to a select number of amino acids along the length of the primary peptide chain. Thus, many naturally occurring peptides are termed “glycopeptides” or “glycoproteins” or are referred to as “glycosylated” proteins or peptides,

The predominant sugars found on glycoproteins are glucose, galactose, mannose, fucose, N-acetylgalactosamine (GalNAc), N-acetylglucosamine (GlcNAc) and sialic acid (e.g., N-acetylneuraminic acid (NANA or NeuAc, where “Neu” is neuraminic acid) and “Ac” refers to acetyl). The processing of the sugar groups occurs co-translationally in the lumen of the ER and continues in the Golgi apparatus for N-linked glycoproteins.

The glycan structures found in naturally occurring glycopeptides are typically divided into two classes, “N-linked glycans” or “N-linked oligosaccharides” and “O-linked glycans” or “O-linked oligosaccharides”. Peptides expressed in eukaryotic cells typically comprise N-glycans. “N-glycans” are N-glycosylated at an amide nitrogen of an asparagine or an arginine residue in a protein via an N-acetylglucosamine residue. These “N-linked glycosylation sites” occur in the peptide primary structure containing, for example, the amino acid sequence asparagine-X-serine/threonine, where X is any amino acid residue except proline and aspartic acid.

A “glycan-binding molecule” refers to any molecule that is capable of binding to a glycan component of a glycoprotein. Typically, the glycan-binding molecule is glycospecies-specific (or glycospecific) in that it selectively binds the glycan structure of one glycospecies of a glycoprotein but not another, such that it can be used to distinguish different glycospecies of the glycoprotein. Glycan-binding molecules can be natural or synthetic, and include, for example, lectins, glycospecific antibodies, glycospecific aptamers (e.g., RNA aptamer, DNA aptamer, or peptide aptamer), glycospecific peptides, and glycospecific small molecule.

The term “glycospecies” refers to a glycoprotein with a distinct type of glycosylation. The “type” or glycosylation is characterised by the glycans and glycan structures present on the glycoprotein, or by binding affinity to a glycospecific binding Tagent. For example, a glycosylation may comprise fucose-related glycans, mannose-related glycans, sialic acid glycans, etc. A glycoprotein may be characterised as belonging to one, two, three, four, five or more than five different glycospecies (for example, a glycosylation may comprise both a fucose-related glycan, a mannose-related glycan and sialic acid-based glycan).

As used herein, a “healthy” subject is a subject that does not have ovarian cancer. As used herein, a subject with a “benign ovarian mass” refers to a symptomatic subject who does not have ovarian cancer.

The term “indicator” as used herein refers to a result or representation of a result, including any information, number, ratio, signal, sign, mark, or note by which a skilled artisan can estimate and/or determine a likelihood of whether or not a subject has ovarian cancer. In the case of the present invention, the “indicator” may optionally be used together with other clinical characteristics to arrive at a determination that the subject is or is not likely to have ovarian cancer. That such an indicator is “determined” is not meant to imply that the indicator is 100% accurate. The skilled clinician may use the indicator together with other clinical indicia to arrive at a conclusion.

The term “immobilized” means that a molecular species of interest is fixed to a solid support, suitably by covalent linkage. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above-described physicochemical interactions typically occur in interactions between molecules. In particular embodiments, all that is required is that the molecules (e.g., glycan-binding molecules) remain immobilized or attached to a support under conditions in which it is intended to use the support, for example to isolate glycoproteins.

The “level”, “abundance” or “amount” of a biomarker is a detectable level or amount in a sample. These can be measured by methods known to one skilled in the art and also disclosed herein. These terms encompass a quantitative amount or level (e.g., weight or moles), a semi-quantitative amount or level, a relative amount or level (e.g., weight % or mole % within class), a concentration, and the like. Thus, these terms encompass absolute or relative amounts or levels or concentrations of a biomarker in a sample. The expression level or amount of biomarker assessed can be used to determine the response to treatment. In specific embodiments in which the level of a biomarker is “reduced” relative to a reference or control, the reduced level may refer to an overall reduction of any of at least about 10%, 20%, 30%, 40%, 50%, 60%, 70%, 80%, 90%, 95%, 96%, 97%, 98%, 99% or greater, in the level of biomarker (e.g., glycospecies of a glycoprotein), detected by standard art known methods such as those described herein, as compared to a reference sample, reference cell, reference tissue, control sample, control cell, or control tissue. In certain embodiments, reduced level refers to a decrease in level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the level/amount of the respective biomarker in a reference sample, reference cell, reference tissue, control sample, control cell, or control tissue. In certain embodiments in which the level of a biomarker is “about the same” as a reference or control, the level of biomarker varies by less than about 10%, 9%, 8%, 7%, 6%, 5%, 4%, 3%, 2%, 1%, 0.5%, 0.1%, or even less, as compared to the level of biomarker (e.g., glycospecies of a glycoprotein), detected by standard art known methods such as those described herein, in a reference sample, reference cell, reference tissue, control sample, control cell, or control tissue. Similarly, the term “underexpressed” and the like refer to a downward deviation in the level of expression of an ovarian cancer biomarker as compared to a baseline expression level of a corresponding ovarian cancer biomarker in a control sample. The term “overexpressed” refers to an upward deviation in the level of glycospecies as compared to a baseline expression level of a corresponding glycospecies in a control sample.

By “obtained” is meant to come into possession. Samples so obtained include, for example, nucleic acid extracts or polypeptide extracts isolated or derived from a particular source. For instance, the extract may be isolated directly from a biological fluid or tissue of a subject.

As used herein, the term “predetermined threshold” refers to a value, above or below which, indicates the presence of disease, such as ovarian cancer, or a healthy condition. For example, for the purposes of the present invention, a predetermined threshold may represent the level of a particular glycospecies of a glycoprotein, or the ratio of the level of a particular glycospecies of a glycoprotein to the total glycoprotein level, in a sample from an appropriate control subject, such as a healthy subject or a subject with benign ovarian neoplasm, or in pooled samples from multiple control subjects or medians or averages of multiple control subjects. Thus, a level or ratio above or below the threshold indicates the presence of ovarian cancer, as taught herein. In other examples, a predetermined threshold may represent a value larger or smaller than the level or ratio determined for a control subject so as to incorporate a further degree of confidence that a level or ratio above or below the predetermined threshold is indicative of the presence of disease, such as ovarian cancer. For example, the predetermined threshold may represent the average or median level of a glycospecies in a group of control subjects, plus or minus 1, 2, 3 or more standard deviations. Those skilled in the art can readily determine an appropriate predetermined threshold based on analysis of biological samples from appropriate control subjects.

The term “receiver operating characteristic (ROC) curves” means a graphical measure of sensitivity (y-axis) vs. 1-specificity (x-axis) for a clinical test. An important measure of the accuracy of the clinical test is the area under the ROC curve value (AUC value). If this area is equal to 1.0 then this test is 100% accurate because both the sensitivity and specificity are 1.0, so there are no false positives and no false negatives. On the other hand, a test that cannot discriminate that is the diagonal line from 0,0 to 1,1. The ROC area for this line is 0.5. ROC curve areas (AUC-values) are typically between 0.5 and 1.0, but also ROC values below 0.5 can—according to information theory—be as good, if the result is interpreted inversely. Therefore, according to the present invention an AUC-value close to 1 (e.g., 0.95) represents the same good measure for a clinical test as an AUC-value close to 0 (e.g., 0.05).

The terms “sample”, “biological sample”, and the like mean a material known or suspected of containing one or more glycospecies or other ovarian cancer biomarkers. A test sample can be used directly as obtained from the source or following a pretreatment to modify the character of the sample. The sample is suitably derived from blood, serum or plasma fractions, including cell fractions (e.g., comprising tumour cells) or lysates thereof, cell-free or cell-depleted fractions, and the like. The sample can be treated prior to use, such as diluting viscous fluids, and the like. Methods of treatment can involve filtration, distillation, extraction, concentration, inactivation of interfering components (e.g., inhibiting nucleases such as RNAses and DNAses), the addition of reagents, and the like.

The term “solid support” as used herein refers to a solid inert surface or body to which a molecular species, such as a nucleic acid and polypeptides can be immobilized. Non-limiting examples of solid supports include magnetic beads, glass surfaces, plastic surfaces, latex, dextran, polystyrene surfaces, polypropylene surfaces, polyacrylamide gels, gold surfaces, and silicon wafers. In some embodiments, the solid supports are in the form of membranes, chips or particles. For example, the solid support may be a glass surface (e.g., a planar surface of a flow cell channel). In some embodiments, the solid support may comprise an inert substrate or matrix which has been “functionalized”, such as by applying a layer or coating of an intermediate material comprising reactive groups which permit covalent attachment to molecules such as polynucleotides. By way of non-limiting example, such supports can include polyacrylamide hydrogels supported on an inert substrate such as glass. The molecules (e.g., polynucleotides) can be directly covalently attached to the intermediate material (e.g., a hydrogel) but the intermediate material can itself be non-covalently attached to the substrate or matrix (e.g., a glass substrate). The support can include a plurality of particles or beads each having a different attached molecular species.

The terms “subject”, “individual” or “patient”, used interchangeably herein, refer to any animal subject, particularly a mammalian subject, more particularly a human subject. In some embodiments, the subject presents with clinical signs of a condition as defined herein. As used herein, the term “clinical sign”, or simply “sign”, refers to objective evidence of a disease present in a subject. Symptoms and/or signs associated with diseases referred to herein and the evaluation of such signs are routine and known in the art. Examples of signs of disease vary depending upon the disease. Signs of ovarian cancer may include tumourigenesis, metastasis, angiogenesis. Typically, whether a subject has a disease, and whether a subject is responding to treatment, may be determined by evaluation of signs associated with the disease.

The terms “treat” and “treating” as used herein, unless otherwise indicated, refer to both therapeutic treatment and prophylactic or preventative measures, wherein the object is to prevent, either partially or completely, ameliorate or slow down (lessen) the targeted condition or disorder (e.g., ovarian cancer), or one or more symptom associated therewith. The terms are also used herein to denote delaying the onset of, inhibiting (e.g., reducing or arresting the growth of), alleviating the effects of or prolonging the life of a patient suffering from, cancer, in particular, ovarian cancer. Those in need of treatment include those diagnosed with the disorder, those suspected of having the disorder, those predisposed to have the disorder as well as those in whom the disorder is to be prevented. Hence, the subject to be treated herein may have been diagnosed as having the disorder or may be predisposed or susceptible to the disorder. In some embodiments, treatment refers to the eradication, removal, modification, or control of primary, regional, or metastatic cancer tissue that results from the administration of one or more therapeutic agents according to the methods of the invention. In other embodiments, such terms refer to the minimising or delaying the spread of cancer resulting from the administration of one or more therapeutic agents to a subject with such a disease. In other embodiments, such terms refer to elimination of disease-causing cells. The term, “treatment” as used herein, unless otherwise indicated, refers to the act of treating.

As used herein, the term “treatment regimen” encompasses natural substances and pharmaceutical agents (i.e., “drugs”) as well as any other treatment regimen including but not limited to chemotherapy, radiotherapy, proton therapy, immunotherapy, hormone therapy, phototherapy, cryotherapy, cryosurgery, toxin therapy or pro-apoptosis therapy, high intensity focused ultrasound, dietary treatments, physical therapy or exercise regimens, surgical interventions, and combinations thereof.

Those skilled in the art will appreciate that the aspects and embodiments described herein are susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

It will be appreciated that the terms used herein and associated definitions are used for the purpose of explanation only and are not intended to be limiting.

### 2. Biomarkers for Ovarian Cancer and Uses Therefore

The present invention concerns methods, compositions, solid supports and kits for assessing the likelihood of a subject having ovarian cancer. Thus, the methods, compositions, apparatus and kits can be used to stratify subjects into those who are likely to have ovarian cancer and those that are unlikely to have ovarian cancer. In some embodiments, the ovarian cancer is a high grade serous ovarian cancer (HGSOC).

2.1 Biomarkers that Distinguish Subjects with Ovarian Cancer from Healthy Subjects

Glycosylation is a post-translational modification on proteins secreted to the bloodstream that can be altered during the development and progression of a cancer, such as ovarian cancer, or a precancerous condition. As a result, the same blood glycoprotein may be detected both before and after oncogenic transformation, but the glycosylation of the glycoprotein before and after oncogenic transformation may be different.

Differences in the type of glycosylation include the removal of a glycan component, the addition of a glycan component, a change in the glycan component such as the substitution of one glycan component for another, the change in the branching of glycans, and the rearrangement of one or more glycan components on the glycoprotein, as where a glycan component is shifted from one position on the polypeptide sequence to another. Differential glycosylation can be detected using any of method known in the art, including but not limited to, methods that detect binding of a particular type of glycosylation to a glycan-binding molecule, such as a lectin, glycospecific antibody or glycospecific aptamer, as farther described herein, that is selective and/or specific for the particular type of glycosylation. Differences in glycosylation can also be detected spectroscopically. For example, mass spectrometry can be used to characterise the glycan component and distinguish between different types of glycosylation.

The present invention is predicated in part on the identification of serum glycoproteins that are differentially glycosylated in subjects with ovarian cancer, benign ovarian neoplasm and in healthy patients. Accordingly, as described herein, detection of the levels of one or more of these different types of glycosylation on particular glycoproteins (i.e., one or more glycospecies) in a biological sample, such as a blood, serum or plasma sample, from a subject can be used to determine whether the subject has ovarian cancer or is healthy (i.e., does not have ovarian cancer). The glycospecies can thus be considered biomarkers for ovarian cancer.

In some instances, the present invention provides a method comprising determining the ratio of a level of a glycospecies of a glycoprotein to the total level of the glycoprotein in the sample, which glycospecies is differentially expressed between ovarian cancer and healthy control (i.e., subjects that are known not to have ovarian cancer), and determining a likelihood of the subject having or not having an ovarian cancer based on whether the ratio is above or below a predetermined threshold. Monitoring the levels or ratios of one or more of the glycospecies identified herein can also be used to monitor the effectiveness and/or efficacy of a treatment regimen. For example, levels or ratios of a particular glycospecies identified herein as being increased or decreased in subjects with ovarian cancer compared to a healthy control group (i.e., subject(s) known not to have ovarian cancer) can be monitored during or after treatment. A change in the level or ratio of one or more glycospecies in the subject over time to be more similar to those levels or ratios observed in control subjects indicates that the disease has regressed. Conversely, a change over time in the level or ratio of one or more glycospecies in the subject to be less similar to those levels or ratios observed in control subjects may indicate that the disease has progressed and/or the treatment regimen as not been effective at treating the ovarian cancer. Methods of monitoring the disease progression of disease by assessing levels or ratios of one or more glycospecies are thus also useful in assessing the efficacy of treatment, e.g., for assessing whether the treatment has resulted in a regression of disease.

The glycoproteins identified herein as being differentially glycosylated in subjects with ovarian cancer are selected from: alpha-1-acid glycoprotein 1 (A1AG1), alpha-1-acid glycoprotein 2 (A1AG2), alpha-1-antitrypsin (A1AT), alpha-1-antichymotrypsin (AACT), alpha-1-microglobulin (AMBP), alpha-2-antiplasmin (A2AP), alpha-2-HS-glycoprotein (FETUA), alpha-2-macroglobulin (A2MG), angiotensinogen (ANGT), carboxypeptidase N subunit 2 (CPN2), cholinesterase (CHLE), coagulation factor X (FA10), coagulation factor XII (FA12), complement C1q subcomponent subunit C (C1QC), complement component 2 (CO2), complement component C9 (CO9), complement factor B (CFAB), complement factor I (CFAI), corticosteroid-binding globulin (CBG), fibrinogen alpha (FIBA), galectin-3-binding protein (LG3BP), gelsolin (GELS), haptoglobin (HPT), haptoglobin-related protein (HPTR), hemopexin (HEMO), heparin cofactor 2 (HEP2), inter-alpha-trypsin inhibitor heavy chain 1 protein (ITIH1), inter-alpha-trypsin inhibitor heavy chain 2 protein (ITIH2), inter-alpha-trypsin inhibitor heavy chain 3 protein (ITIH3), inter-alpha-trypsin inhibitor heavy chain 4 protein (ITIH4), insulin-like growth factor-binding protein 3 (IBP3), insulin-like growth factor-binding protein complex acid labile subunit (ALS), kininogen-1 (KNG1), lumican (LUM), N-acetylmuramoyl-L-alanine amidase (PGRP2), plasma protease C1 inhibitor (IC1), plasminogen (PLMN), prothrombin (THRB), serotransferrin (TRFE), and serum paraoxonase/arylesterase 1 (PON1).

In some embodiments, the one or more glycoprotein is selected from alpha-1-antitrypsin (A1AT), alpha-2-macroglobulin (A2MG), complement component C9 (CO9), insulin-like growth factor-binding protein 3 (IBP3), insulin-like growth factor-binding protein complex acid labile subunit (ALS), inter-alpha-trypsin inhibitor heavy chain H3 (ITIH3), serum paraoxonase/arylesterase 1 (PON1), haptoglobin (HPT), and alpha-1-antichymotrypsin (AACT).

The various types of glycosylation associated with the above serum glycoproteins exhibit different lectin-binding properties resulting from the different glycan structures on the proteins. These types of glycosylation include, for example, glycosylations that bind to lectins having a general reactivity with α/β-D-Galactose, including Bauhinia purpurea lectin (BPL, known to bind at least Galβ1-3GaINAc), Etythrina cristagalli agglutinin (ECA, known to bind at least Gal β1-4GlcNAc), and jacalin (JAC, known to bind at least Gala1-6GalNAc and Galβ1-3GaINAc), lectins having a general reactivity with D-N-Acetylgalactosamine, including soybean agglutinin (SBA, known to bind to at least GalNAca1-3Gal), Helix pomalia agglutinin (HPA, known to bind to at least α-GalNAc), Wisteria florihunda agglutinin (WFA, known to bind to at least GalNAca1-6Gal and GalNAca1-3GalNAc), Datum stramonium lectin (DSA, known to bind to at least β1-4GlcNAc oligomers), Helix aspersa agglutinin (HAA, known to bind to at least α-GlcNAc and α-GalNAc), Solanum tuberosum lectin (STL, known to bind to at least GlcNAcβ1-4GlcNAc oligomers), and wheat germ agglutinin (WGA, known to bind to at least GlcNAcβ1-4GlcNAc and Neu5Ac), lectins having a general reactivity with D-Mannose, including concanavalin A (ConA, known to bind to at least α-Man, α-Glc, and α-GlcNAc), Gaianthus nivalis lectin (GNL, known to bind to at least Mana1-3Man) and Narcissus pseadonarcissm (NPL, known to bind to at least Mana1-6Man), lectins having a general reactivity with α-L-Fucose, including Aleuria aurantia lectin (AAL, known to bind to at least Fuca1-2,-3,-6 linked), Pisum sativum agglutinin (PSA, known to bind to at least Fuca1-6GlcNAc of N-linked glvcans) and Ulex europeus agglutinin-I (UEA, known to bind to at least Fucα1-2Galβ1-4GlcNAc), lectins having a general reactivity with sialic acid, including Maackia amurensis agglutinin-II (MAA, known to bind to at least Neu5Acα2-3Galβ1-3 linkages) and Sambucus nigra agglutinin (SNA, known to bind to at least Neu5Acα2-6 linkages), and lectins having a general reactivity with complex specificities, including erythroagglutinating phytohemagglutinin (EPHA, known to bind to at least bisecting GlcNAc) and leukoagglutinating phytohemagglutinin (L-PHA, known to bind to at least tri/tetra-antennary β1-6GlcNAc).

Accordingly, the lectin-binding properties of the glycoproteins provided herein indicate the type of glycosylation. For example, an AAL-binding glycoprotein, such as an AAL-binding glycospecies of alpha-1-acid glycoprotein 2 (A1AG2), is presumed to be fucosylated type, possibly containing Fuca1-2,-3,-6 linked oligosaccharides, while an SNA-binding glycoprotein, such as a SNA-binding glycospecies of alpha-1-acid glycoprotein 2 (A1AG2) likely contains Neu5Acα2-6 linkages. The identity of the glycans on the glycoprotein biomarkers described herein can be more precisely determined using standard methods well known in the art, such as, for example, mass spectrometry, high-pressure liquid chromatography, nuclear magnetic resonance, correlation spectroscopy, gas-liquid chromatography, or gas chromatography.

As described herein, the levels of the various types of glycosylation present on a single glycoprotein in a sample, such as a blood, serum or plasma sample, from subjects with ovarian cancer is different to that of subjects with benign ovarian neoplasm condition or healthy subjects. Accordingly, the glycospecies provided herein are useful as biomarkers in methods for detecting and monitoring the recurrence of ovarian cancer, and related methods and uses.

Accordingly, a determination of the likelihood of whether a subject has ovarian cancer or is healthy and does not have ovarian cancer can be made by assessing the level of one or more of these glycospecies in a biological sample from the subject, such as a serum, plasma or blood sample, and comparing it to the level of the same glycoprotein of the same glycosylation type in a corresponding sample from a healthy control subject (i.e. a subject that is known to not have ovarian cancer) or samples from multiple healthy control subjects, wherein an increase or decrease indicates that the subject has ovarian cancer. In some instances, the level of the glycospecies is compared to a predetermined level or threshold, wherein an increase or decrease in the level of the subject compared to the threshold indicates that the subject has ovarian cancer. The predetermined threshold may be calculated based on the level of the same glycospecies in a corresponding sample from a healthy control subject or from a group of healthy subjects, such that a level of the glycospecies above or below the predetermined level indicates that the subject has ovarian cancer. In some instances, the ratio of the level of one or more glycospecies to the total level of the glycoprotein is also increased or decreased in a subject with ovarian cancer compared to a healthy control subject or a group of healthy control subjects, and can thus also be used to determine the presence of ovarian cancer. Where two or more types of glycosylation are assessed for a single glycoprotein, a separate ratio for each glycospecies can be determined. Alternatively, a single ratio of the combined levels of the two or more types of glycosylation with a single glycoprotein to the total level of the glycoprotein can be determined.

Preferably, the individual glycospecies that are differentially expressed between subjects with ovarian cancer and healthy subjects in a subject are selected from the glycospecies identified in TABLE 1:

TABLE 1
DIFFERENTLY ABUNDANT GLYCOSPECIES
BETWEEN OVARIAN CANCER AND HEALTHY

| | Glycoprotein | |
|---|---|---|
| Lectin | Overexpressed | Underexpressed |
| AAL | A1AG1, A1AG2, A1AT, A1BG, | A2MG, AMBP, C1QC, CBG, CHLE, |
| | AACT, CERU, CFAB, CFAI, | FETUA, GELS, IBP3, ITIH1, ITIH2, |
| | CO2, CO9, FA5, FA10, FIBA, | PON1, TRFE |
| | FIBB, HPT, HPTR, IC1, ITIH3, | |
| | ITIH4, PEDF, VTDB | |
| SNA | A1AG1, A1AT, AACT, CFAB, | A1AG2, A2AP, A2MG, ALS, AMBP, |
| | CO9, FA10, FIBA, FIBB, HPT, | ANGT, APOB, C1QC, CBG, CHLE, |
| | HPTR, IC1, ITIH3 | CO6, CO7, CPN2, FA10, FA12, |
| | | FETUA, , GELS, HEMO, HEP2, |
| | | IBP3, ITIH1, ITIH2, KNG1, PLMN, |
| | | PON1, PGRP2, THRB, TRFE |
| STL | CO9, FIBA, FIBB, HPT, HPTR, | A2MG, A2AP, ALS, AMBP, ANGT, |
| | ITIH3, LUM | C1QC, CBG, CHLE, CPN2, FA10, |
| | | FA12, FETUA, GELS, HEP2, |
| | | HPTR, IBP3, ITIH1, ITIH2, ITIH4, |
| | | LG3BP, KNG1, PGRP2, PLMN, |
| | | PON1, TRFE |

Accordingly, a determination of the likelihood of whether a subject has ovarian cancer or is healthy (i.e., is not likely to have ovarian cancer) can be made by assessing the level of one or more glycospecies in a biological sample from the subject, such as a serum, plasma or blood sample, and comparing it to the level of the same one or more glycospecies in a corresponding sample from a healthy control subject (i.e. a subject that is known to not have ovarian cancer) or samples from multiple healthy control subjects, wherein an increase or decrease indicates that the subject has ovarian cancer. In some instances, the level of the one or more glycospecies is compared to a predetermined level or threshold, wherein an increase or decrease in the level of the subject compared to the threshold indicates that the subject has ovarian cancer. The predetermined threshold may be calculated based on the level of the same glycospecies in a corresponding sample from a healthy control subject or from a group of healthy subjects, such that a level of the glycospecies above or below the predetermined level indicates that the subject has ovarian cancer. In some instances, the ratio of the level of a glycospecies to the total level of the corresponding glycoprotein is also increased or decreased in a subject with ovarian cancer compared to a healthy control subject or a group of healthy control subjects, and can thus also be used to determine the likelihood of the presence of ovarian cancer. Where two or more glycospecies are assessed for a single glycoprotein, a separate ratio for each glycospecies can be determined. Alternatively, a single ratio of the combined levels of the glycospecies of the single glycoprotein to the total level of the glycoprotein can be determined.

Illustrative glycospecies that have increased levels in subjects with ovarian cancer compared to healthy subjects include, for example, those glycospecies identified in Table 1 as being overexpressed in subjects with ovarian cancer. Thus, a determination that a subject has an increased level of one or more of these types of glycosylation compared to a healthy control subject or compared to a predetermined threshold indicates that the subject has ovarian cancer. Similarly, a determination that a subject has an increased ratio of the level of a glycoprotein with one or more types of glycosylation to the total level of the glycoprotein compared to the ratio in a healthy control subject or compared to a predetermined threshold indicates that the subject has ovarian cancer.

Illustrative glycospecies of glycoproteins that have decreased levels in subjects with ovarian cancer compared to healthy subjects include, for example, those glycospecies of glycoproteins identified in Table 1 as being underexpressed in subjects with ovarian cancer, Thus, a determination that a subject has a decreased level of one or more of these glycospecies of glycoprotein compared to a healthy control subject or compared to a predetermined threshold indicates that the subject has ovarian cancer. Similarly, a determination that a subject has a decreased ratio of the level of one or more of these glycospecies of glycoprotein to the total level of the glycoprotein compared to the ratio in a healthy control subject or compared to a predetermined threshold indicates that the subject has ovarian cancer.

The levels or ratios of the glycospecies of glycoprotein identified above as being useful biomarkers for ovarian cancer can also be used to monitor the progress of disease in a subject that has ovarian cancer. For example, the progress of ovarian cancer can be assessed or monitored before, during or after treatment by assessing the level or ratio (i.e., the ratio of the level of a glycospecies of glycoprotein to the total level of the glycoprotein) of one of more glycospecies of glycoprotein in samples taken at various time points. Accordingly, the efficacy of treatment can also be assessed by determining the level or ratio of one or more glycospecies of glycoprotein in samples taken at various time points, wherein at least one of those time points is during or after treatment. An increase over time in the level or ratio of one or more of the glycospecies of glycoprotein identified above as being increased in subjects with ovarian cancer compared to healthy subjects indicates that the disease has progressed, while a decrease in one or more of these glycospecies of glycoprotein indicates that the disease has regressed. Conversely, a decrease over time in the level or ratio of one or more of the glycospecies of glycoprotein identified above as being decreased in subjects with ovarian cancer compared to healthy subjects indicates that the disease has progressed, while an increase in one or more of these glycospecies of glycoprotein indicates that the disease has regressed. In instances where the subject has undergone or is undergoing treatment for ovarian cancer, progression of the disease may indicate that such treatment has not been effective, or there is a relapse of disease, while regression of the disease may indicate that such treatment has been at least partially effective.

Accordingly, an increase over time in the level or ratio of one or more of the glycospecies of glycoprotein identified in TABLE 1, as being overexpressed in those subjects which are diagnosed with ovarian cancer indicates that the subject's ovarian cancer has progressed or relapsed over time, while a decrease of any one or more of these glycospecies indicates that the subject's ovarian cancer has regressed.

A decrease over time in the level or ratio of one or more of the glycospecies identified in TABLE 1, as being underexpressed in those subjects diagnosed with ovarian cancer indicates that the subject's ovarian cancer has progressed or relapsed over time, while an increase of any one or more of these glycospecies indicates that the subject's ovarian cancer has regressed.

In some instances, the level or ratio of more than one type of glycosylation with a single glycoprotein identified above is assessed to determine the presence or progression of ovarian cancer in a subject. For example, 2, 3, 4, 5, 6, 7, 8, 9, 10, 15, 20 or more types of glycosylation with a single glycoprotein may be assessed. Thus, a panel of more than one glycospecies may be assessed to determine a glycoprotein's glycosylation profile or signature for a subject. In some embodiments, this profile can be compared to a corresponding profile from a control subject or a group of control subjects to determine the presence or progression of ovarian cancer, wherein a change in the profile resulting from increases or decreases in the levels or ratios of the various types of glycosylation for a single glycoprotein, as described above, indicates the presence or progression of ovarian cancer. In some alternative embodiments a value (e.g., a numeric value) could be determined by the panel, in which the value provides an indication of the likelihood that the subject has, or does not have, ovarian cancer.

2.2 Markers for Distinguishing Ovarian Cancer from Benign Ovarian Condition

As described herein, detection of the levels of one or more of different types of glycosylation on particular glycoproteins (i.e., one or more glycospecies) in a biological sample, such as a blood, serum or plasma sample, from a subject can be used to distinguish between a subject that has ovarian cancer and a subject with a benign ovarian neoplasm.

In some instances, the present invention provides a method comprising determining the ratio of a level of a glycospecies of a glycoprotein to the total level of the glycoprotein in the sample, which glycospecies is differentially expressed between ovarian cancer and benign ovarian neoplasm, and determining a likelihood of the subject having or not having an ovarian cancer based on whether the ratio is above or below a predetermined threshold.

The glycoproteins identified herein as being differentially glycosylated in subjects with ovarian cancer as compared to subjects with a benign ovarian neoplasm are selected from alpha-1-acid glycoprotein 1 (A1AG1), alpha-1-acid glycoprotein 2 (A1AG2), alpha-1-antitrypsin (A1AT), alpha-1-B glycoprotein (A1BG), alpha-2-antiplasmin (A2AP), alpha-2-macroglobulin (A2MG), alpha-1-antichymotrypsin (AACT), insulin-like growth factor-binding protein complex acid labile subunit (ALS), alpha-1-microglobulin (AMBP), angiotensinogen (ANGT), apolipoprotein B (APOB), ceruloplasmin (CERU), complement C1q subcomponent subunit C (C1QC), corticosteroid-binding globulin (CBG), cholinesterase (CHLE), complement factor B (CFAB), complement factor I (CFAI), complement component 2 (CO2), complement component 6 (CO6), complement component 7 (CO7), complement component 9 (CO9), carboxypeptidase N subunit 2 (CPN2), coagulation factor V (FA5), coagulation factor X (FA10), coagulation factor XII (FA12), alpha-2-HS-glycoprotein (FETUA), fibrinogen alpha (FIBA), fibrinogen beta (FIBB), gelsolin (GELS), hemopexin (HEMO), heparin cofactor 2 (HEP2), haptoglobin (HPT), haptoglobin-related protein (HPTR), insulin-like growth factor-binding protein 3 (IBP3), plasma protease C1 inhibitor (IC1), inter-alpha-trypsin inhibitor heavy chain 1 protein (ITIH1), inter-alpha-trypsin inhibitor heavy chain 2 protein (ITIH2), inter-alpha-trypsin inhibitor heavy chain 3 protein (ITIH3), inter-alpha-trypsin inhibitor heavy chain 4 protein (ITIH4), kininogen-1 (KNG1), galectin-3 binding protein (LG3BP), pigment epithelium-derived factor (PEDF), plasminogen (PLMN), paraoxonase/arylesterase 1 (PON1), N-acetylmuramoyl-L-alanine amidase (PGRP2), prothrombin (THRB), serotransferrin (TRFE), and vitamin D-binding protein (VTDB).

The various types of glycosylation associated with the above serum glycoproteins exhibit different lectin-binding properties resulting from the different glycan structures on the proteins. These types of glycosylation include, for example, glycosylations that bind to lectins having a general reactivity with α/β-D-Galactose, including Bauhinia purpurea lectin (BPL, known to bind at least Galβ1-3GaINAc), Etythrina cristagalli agglutinin (ECA, known to bind at least Galβ1-4GlcNAc), and jacalin (JAC, known to bind at least Gala1-6GalNAc and Galβ1-3GaINAc), lectins having a general reactivity with D-N-Acetylgalactosamine, including soybean agglutinin (SBA, known to bind to at least GalNAca1-3Gal), Helix pomalia agglutinin (HPA, known to bind to at least α-GalNAc), Wisteria florihunda agglutinin (WFA, known to bind to at least GalNAca1-6Gal and GalNAca1-3GalNAc), Datum stramonium lectin (DSA, known to bind to at least B1-4GlcNAc oligomers), Helix aspersa agglutinin (HAA, known to bind to at least α-GlcNAc and α-GalNAc), Solanum tuberosum lectin (STL, known to bind to at least GlcNAcβ1-4GlcNAc oligomers), and wheat germ agglutinin (WGA, known to bind to at least GlcNAcβ1-4GlcNAc and Neu5Ac), lectins having a general reactivity with D-Mannose, including concanavalin A (ConA, known to bind to at least α-Man, α-Glc, and α-GlcNAc), Gaianthus nivalis lectin (GNL, known to bind to at least Mana1-3Man) and Narcissus pseadonarcissm (NPL, known to bind to at least Mana1-6Man), lectins having a general reactivity with α-L-Fucose, including Aleuria aurantia lectin (AAL, known to bind to at least Fuca1-2,-3,-6 linked), Pisum sativum agglutinin (PSA, known to bind to at least Fuca1-6GlcNAc of N-linked glvcans) and Ulex europeus agglutinin-I (UEA, known to bind to at least Fuca1-2Galβ1-4GlcNAc), lectins having a general reactivity with sialic acid, including Maackia amurensis agglutinin-II (MAA, known to bind to at least Neu5Acα2-3Galβ1-3 linkages) and Sambucus nigra agglutinin (SNA, known to bind to at least Neu5Acα2-6 linkages), and lectins having a general reactivity with complex specificities, including erythroagglutinating phytohemagglutinin (EPHA, known to bind to at least bisecting GlcNAc) and leukoagglutinating phytohemagglutinin (L-PHA, known to bind to at least tri/tetra-antennary β1-6GlcNAc).

Accordingly, the lectin-binding properties of the glycoproteins provided herein indicate the type of glycosylation. For example, an AAL-binding glycoprotein, such as an AAL-binding glycospecies of alpha-1-acid glycoprotein 1 (A1AG1), is presumed to be fucosylated type, possibly containing Fuca1-2,-3,-6 linked oligosaccharides, while an SNA-binding glycoprotein, such as a SNA-binding glycospecies of alpha-1-antitrypsin (A1AT) likely contains Neu5Acα2-6 linkages. The identity of the glycans on the glycoprotein biomarkers described herein can be more precisely determined using standard methods well known in the art, such as, for example, mass spectrometry, high-pressure liquid chromatography, nuclear magnetic resonance, correlation spectroscopy, gas-liquid chromatography, or gas chromatography.

In some embodiments, the presence, absence, or amount of glycospecies of a glycoprotein is measured using one or more peptides from TABLE 4 and/or TABLE 5. In some embodiments of this type, an increase in the amount of a peptide with an amino acid sequence set forth in any one of SEQ ID NOs: 1-46 is indicative of ovarian cancer being present in the subject. Similarly, a decrease in the amount of a peptide with an amino acid sequence set forth in any one of SEQ ID NOs: 47-123 is indicative of ovarian cancer being present in the subject (as compared to a control or reference sample being a subject that does not have ovarian cancer, for example, a healthy subject or a subject with a benign ovarian neoplasm).

As described herein, the levels of the various types of glycosylation present on a single glycoprotein in a sample, such as a blood, serum or plasma sample, from subjects with ovarian cancer is different to that of subjects with benign ovarian neoplasm. Accordingly, the glycospecies provided herein are useful as biomarkers in methods for detecting and monitoring the recurrence of ovarian cancer, and related methods and uses.

Accordingly, a determination of the likelihood of whether a subject has ovarian cancer or a benign ovarian neoplasm can be made by assessing the level of one or more of these glycospecies in a biological sample from the subject, such as a serum, plasma or blood sample, and comparing it to the level of the same glycoprotein of the same glycosylation type in a corresponding sample from a subject with a benign ovarian neoplasm (i.e. a subject that is known to not have ovarian cancer and has a benign ovarian neoplasm) or samples from multiple subjects with benign ovarian neoplasm, wherein an increase or decrease indicates that the subject has ovarian cancer. In some instances, the level of the glycospecies is compared to a predetermined level or threshold, wherein an increase or decrease in the level of the subject compared to the threshold indicates that the subject has ovarian cancer. The predetermined threshold may be calculated based on the level of the same glycospecies in a corresponding sample from a subject with a benign ovarian neoplasm or from a group of subjects with a benign ovarian neoplasm, such that a level of the glycospecies above or below the predetermined level indicates that the subject has ovarian cancer. In some instances, the ratio of the level of one or more glycospecies to the total level of the glycoprotein is also increased or decreased in a subject with ovarian cancer compared to a subject with a benign ovarian neoplasm or a group of control subjects with a benign ovarian neoplasm, and can thus also be used to determine the presence of ovarian cancer. Where two or more types of glycosylation are assessed for a single glycoprotein, a separate ratio for each glycospecies can be determined. Alternatively, a single ratio of the combined levels of the two or more types of glycosylation with a single glycoprotein to the total level of the glycoprotein can be determined.

Preferably, the individual glycospecies that are differentially expressed between subjects with ovarian cancer and subjects with a benign ovarian neoplasm are selected from the glycospecies identified in TABLE 2:

TABLE 2
DIFFERENTLY ABUNDANT GLYCOSPECIES BETWEEN
OVARIAN CANCER AND BENIGN OVARIAN NEOPLASM

| | Glycoprotein | |
|---|---|---|
| Lectin | Overexpressed | Underexpressed |
| AAL | A1AG1, A1AG2, A1AT, A1BG, | A2MG, GELS, IBP3, ITIH2 |
| | AACT, CERU, CFAB, CFAI, | |
| | CO2, CO9, FA5, FA10, FIBA, | |
| | FIBB, HPT, HPTR, IC1, ITIH3, | |
| | ITIH4, PEDF, VTDB | |
| SNA | A1AT, AACT, CO9, FA10, | A1AG2, A2AP, A2MG, ALS, AMBP, |
| | FIBA, FIBB, HPT, HPTR, | ANGT, APOB, C1QC, CBG, CHLE, |
| | ITIH3 | CO6, CO7, CPN2, FA10, FA12, |
| | | FETUA, GELS, IBP3, ITIH1, ITIH2, |
| | | KNG1, PLMN, PON1, PGRP2, |
| | | THRB, TRFE |
| STL | CO9, FIBB, HPT, HPTR | A2MG, A2AP, ALS, AMBP, ANGT, |
| | | CBG, CHLE, CPN2, FA10, FETUA, |
| | | GELS, HPTR, IBP3, ITIH1, ITIH2, |
| | | KNG1, PLMN, PON1, TRFE |

Accordingly, a determination of the likelihood of whether a subject has ovarian cancer or a benign ovarian neoplasm (i.e., is not likely to have ovarian cancer) can be made by assessing the level of one or more glycospecies in a biological sample from the subject, such as a serum, plasma or blood sample, and comparing it to the level of the same one or more glycospecies in a corresponding sample from a control subject with a benign ovarian neoplasm or samples from multiple control subjects with benign ovarian neoplasm, wherein an increase or decrease indicates that the subject has ovarian cancer. In some instances, the level of the one or more glycospecies is compared to a predetermined level or threshold, wherein an increase or decrease in the level of the subject compared to the threshold indicates that the subject has ovarian cancer. The predetermined threshold may be calculated based on the level of the same glycospecies in a corresponding sample from a control subject with benign ovarian neoplasm or from a group of subjects with benign ovarian neoplasm, such that a level of the glycospecies above or below the predetermined level indicates that the subject has ovarian cancer. In some instances, the ratio of the level of a glycospecies to the total level of the corresponding glycoprotein is also increased or decreased in a subject with ovarian cancer compared to a control subject with a benign ovarian neoplasm or a group of control subjects with benign ovarian neoplasm, and can thus also be used to determine the likelihood of the presence of ovarian cancer. Where two or more glycospecies are assessed for a single glycoprotein, a separate ratio for each glycospecies can be determined. Alternatively, a single ratio of the combined levels of the glycospecies of the single glycoprotein to the total level of the glycoprotein can be determined.

Illustrative glycospecies that have increased levels in subjects with ovarian cancer compared to subjects with a benign ovarian neoplasm include, for example, those glycospecies identified in TABLE 2 as being overexpressed in subjects with ovarian cancer. Thus, a determination that a subject has an increased level of one or more of these types of glycosylation compared to a control subject with a benign ovarian neoplasm or compared to a predetermined threshold indicates that the subject has ovarian cancer. Similarly, a determination that a subject has an increased ratio of the level of a glycoprotein with one or more types of glycosylation to the total level of the glycoprotein compared to the ratio in a control subject with a benign ovarian neoplasm or compared to a predetermined threshold indicates that the subject has ovarian cancer.

Illustrative glycospecies of glycoproteins that have decreased levels in subjects with ovarian cancer compared to subjects with a benign ovarian neoplasm include, for example, those glycospecies of glycoproteins identified in TABLE 2 as being underexpressed in subjects with ovarian cancer, Thus, a determination that a subject has a decreased level of one or more of these glycospecies of glycoprotein compared to a control subject with a benign ovarian neoplasm or compared to a predetermined threshold indicates that the subject has ovarian cancer. Similarly, a determination that a subject has a decreased ratio of the level of one or more of these glycospecies of glycoprotein to the total level of the glycoprotein compared to the ratio in a control subject with benign ovarian neoplasm or compared to a predetermined threshold indicates that the subject has ovarian cancer.

In some instances, the level or ratio of more than one type of glycosylation of a single glycoprotein identified above is assessed to distinguish between ovarian cancer in a subject and a subject with a benign ovarian neoplasm. For example, 2, 3, 4, 5, 6, 7, 8, 9, 10, 15, 20 or more types of glycosylation with a single glycoprotein may be assessed. Thus, a panel of more than one glycospecies may be assessed to determine a glycoprotein's glycosylation profile or signature for a subject. In some embodiments, this profile can be compared to a corresponding profile from a control subject or a group of control subjects to determine the presence or progression of ovarian cancer, wherein a change in the profile resulting from increases or decreases in the levels or ratios of the various types of glycosylation for a single glycoprotein, as described above, indicates the presence of ovarian cancer. In some alternative embodiments a value (e.g., a numeric value) could be determined by the panel, in which the value provides an indication of the likelihood that the subject has, or does not have, ovarian cancer.

### 2.3Methods for Assessing the Levels of Biomarkers

The levels of glycospecies identified herein as being useful biomarkers for detecting the likelihood of the presence or absence of, or monitoring the recurrence of, ovarian cancer, can be assessed by any method known in the art. Such methods include, but are not limited to, methods that detect specific glycospecies, using a binding reagent such as a lectin, glycospecific antibody, or glycospecific aptamer that is selective. These methods may include mass spectrometry, western blots, ELISAs and other immunoassay-based techniques with varying readout methods. Spectroscopic methods can also be used to assess the level of a glycospecies in a sample.

The level of one or more glycospecies as described herein is assessed in a biological sample from a subject. Most typically, the biological sample is a blood, serum, plasma or blood fraction sample, although other types of samples are contemplated. The sample may be obtained from the subject before or after diagnosis of ovarian cancer. For example, in the methods of the present invention that are used to detect ovarian cancer, the sample may be obtained from a subject. In the methods of the present invention that are used to monitor the recurrence of ovarian cancer, the sample is obtained from the subject after they have been exposed to a treatment regimen prescribed for the treatment of ovarian cancer. For example, in some instances, the subject has been diagnosed with ovarian cancer, then been assessed as having cleared the disease before the sample is taken to assess the levels of one or more glycospecies. In instances where the subject has been diagnosed as having or having had ovarian cancer, the subject may have undergone or be undergoing treatment, such as surgical or medical treatment. One or more samples can be taken from the subject at one or more time points. For example, to monitor the progress of disease, at least two samples are taken at two different time points, so as to compare the levels of one or more glycoproteins over time. In particular examples, 2, 3, 4, 5, 6, 7, 8, 9, 10 or more samples are taken from the subject over a period of days, weeks, months or years. In some embodiments the subject suffers with one or more symptoms of ovarian cancer, or alternatively, the subject may be asymptomatic. In some embodiments, the subject may have one or more risk factors for ovarian cancer (e.g., age, hereditary, etc).

In particular embodiments of the methods of the invention, the level of a glycospecies is assessed by detecting the binding of the glycospecies to an appropriate glycan-binding molecule. In one example, the glycan-binding molecule is a lectin. Lectins are proteins or glycoproteins that bind to all or part of a glycan structure. A lectin may bind to a specific glycan moiety that is part of a glycoprotein or another glycan-containing molecule such as a glycolipid, glycophosphatidylinositol or glycosaminoglycan. Advantageously, the high affinity of a lectin for a particular glycan moiety facilitates the precipitation, isolation and/or detection of glycoproteins with a particular single type of glycosylation from or in a biological sample by specifically binding to those types of glycosylation.

Lectins useful for binding glycospecies of glycoprotein are described herein so as to determine the levels of the glycospecies in a sample include Bauhinia purpurea lectin (BPL), Erythrina eristagalli agglutinin (EGA), jacalin (JAC), soybean agglutinin (SBA), Helix pomatia agglutinin (HPA), Wisteria floribunda agglutinin (WFA), Datura stramonium lectin (DSA), Helix aspersa agglutinin (HAA), Solanum tuberosum lectin (STL), wheat germ agglutinin (WGA), concanavalin A (ConA), Galanthus nivalis lectin (GNL), Narcissus pseudonarcissus lectin (NPL), Aleuria aurantia lectin (AAL), Piston sativum agglutinin (PSA), Ulex europeus agglutinin-I (UEA), Maackia amurensis agglutinin-II (MAA), Sambucus nigra agglutinin (SNA), erythroagglutinating phytohemagglutinin (E-PHA), and leukoagglutinating phytohemagglutinin (L-PBA). For example, AAL can be used to detect the levels of any AAE-binding glycoprotein, such as AAL-gelsolin and AAL-binding complement component C9. Similarly, PSA can be used to detect the levels of any PSA-binding glycoprotein, including, but not limited to, PSA-binding complement C5 and PSA-binding retinol-binding protein 4.

Assays and techniques to detect binding of a glycospecies to a lectin are well known in the art, and any such assay or technique can be used in the methods of the present invention. In one example, lectin-magnetic bead array-coupled mass spectrometry (LeMBA-MS), as described by Choi et al. (Electrophoresis (2011) 32, 3564-3575) and also below in the Examples, is used. In other examples, immunoassays that utilise lectin to capture the glycoprotein with a single glycosylation and a glycoprotein-specific antibody to detect and quantify the captured glycoprotein are used. Such assays include, for example, western blots, ELISAs, lectin-AlphaLISA assays, and immunofluorescence. For example, lectin-based ELISAs (or lectin-based immunosorbent assays (LIAs)) may involve coating a surface, such as a multiwell plate, with an antibody specific for the polypeptide backbone of the glycoprotein, then adding the biological sample containing the glycoprotein to form an immobilised complex. The complex is then contacted with the appropriate biotinylated lectin and detected using streptavidin. In other example, the plate is coated with lectin and the biological sample is added to form lectin-glycoprotein complexes, which are then detected using antibodies specific for the polypeptide backbone of the glycoprotein. These types of techniques are amenable to modification for use in clinical and diagnostic applications that require high sensitivity and accuracy with relatively low cost. For example, automated analysers based on liquid-phase binding can be used to detect and quantify specific glycoproteins in a biological sample, such as a serum sample. Choi et al. (Clinica Chimica Acta (2012) 413:170-174) and Kagebayashi et al. (Anal Biochem (2009) 388;306-311) describe the use of the micro-total analysis systems (μT/AS) immunoassays, performed using fully automated instruments such as the μTASWako® i30 Immunoanalyzer (Wako Pure Chemicals Industries, Ltd) to detect and quantify an LCA-binding glycoprotein of alpha-fetoprotein, and such systems are readily adaptable to detect and quantify the glycoproteins described herein.

Techniques involving the use of electrochemical sensors are also suitable for use in the methods of the invention to assess levels of a glycospecies in a sample by binding the glycospecies to a lectin. Electrochemical sensors include a biorecognition element, such as a lectin, coupled to an electrode transducer surface. The specific interaction of a biological sample containing, for example, a glycospecies, to its corresponding lectin on the electrode surface is detected by electrical current or potential changes occurring at the transducer/biomolecule interface. This type of technique is described by Shah, A.K., Electrochemical detection of glycan and protein epitopes of glycoproteins in serum, Analyst, 2014, 139(22): 5970-6, and is particularly suited to point of care applications. In some alternative embodiments, techniques involving surface-enhanced Raman scattering (SERS) integrated in microfluidic systems can be used. As described in detail in Webster et al., “Development of EndoScreen Chip, a Microfluidic Pre-Endoscopy Triage Test for Esophageal Adenocarcinoma”, Cancers (Basel), 2021 13(12): 2865, the SERS technique is based on Raman scattering of light in proximity to plasmonic structures such as metallic (e.g., gold) nanoparticles. The plasmonic structures enhance the relatively weak Raman signal by multiple orders of magnitude, enabling ultrasensitive biomarker analysis.

Thus, the present invention is also directed to the use of lectins and compositions comprising lectins to detect the levels of one or more glycospecies in a biological sample, and thus uses of lectins to determine the likelihood of the presence or absence of, or monitor the progress of, ovarian cancer in subject. For example, provided are uses of a lectin to determine the level of one or more glycospecies in a sample, comprising contacting the sample with the lectin under conditions that permit binding of the glycospecies to the lectin, and detecting and determining the level of the glycospecies in the sample, wherein a level of the glycospecies above or below a predetermined threshold indicates the presence of ovarian cancer, as described above in section 2. Similarly, lectins can be used to determine the ratio of the level of a glycospecies in a sample to the total level of the glycoprotein, as described above, wherein a ratio of the glycospecies above or below a predetermined threshold indicates the presence of ovarian cancer, as described above in section 2.

In some of the methods of the present invention, a ratio of the level of a glycospecies to the total level of the glycoprotein (i.e., the combined level of all types or glycosylation of the glycoprotein) in a sample is determined. Thus, the methods of the present invention may also require assessing the total level of a glycoprotein in a sample. Any method for determining the level of a glycoprotein, such as the concentration or amount of a glycoprotein, in a sample can be used, and such methods are widely known to those of skill in the art. Exemplary methods include, but are not limited to, immunoassays that utilise glycoprotein-specific antibodies to capture the glycoproteins and a secondary antibody to detect and quantify the captured glycoprotein. Such assays include, for example, western blots, ELISAs and immunofluorescence. The μTAS immunoassays described above are also suitable for detecting and quantifying levels of a glycoprotein, as are techniques involving the use of electrochemical sensors, as described above. In some embodiments, the detection and quantitation of the glycoprotein is performed simultaneously with, and using the same platform, as detection and quantitation of the glycospecies. For example, the levels of a particular glycospecies and the total levels of the glycoprotein can be simultaneously assessed using the μTAS immunoassays referred to above and described by Choi et al (Clinica Chimica Acta (2012) 413:170-174) and Kagebayashi et al. (Anal Biochem (2009) 388:306-311).

In other methods of the invention, a score may be used to integrate the information from multiple glycoprotein biomarker measures. Common methods for multivariate marker selection and modelling include, among others, cluster analysis, discriminant function analysis, factor analysis, machine learning, multidimensional scaling, neural network, principal component, multiple regression analysis.

### 2.4 Deriving Biomarker Values

Biomarker values can be measured biomarker values, which are values of biomarkers directly measured for the subject, or alternatively could be “derived” biomarker values, which are values that have been derived from one or more measured biomarker values, for example by applying a function to the one or more measured biomarker values. As used herein, biomarkers to which a function has been applied are referred to as “derived biomarkers.”

The biomarker values may be determined in any one of a number of ways that are well known in the art. For example, a comprehensive description of biomarker value determination can be found in International Patent Publication No. WO2015/117204, which is incorporated herein by reference in its entirety. In one example, the process of determining biomarker values can include measuring the biomarker values, for example by performing tests on the subject or on sample(s) obtained from the subject.

More typically, however, the step of determining the biomarker values includes having an electronic processing device receive or otherwise obtain biomarker values that have been previously measured or derived. This could include for example, retrieving the biomarker values from a data store such as a remote database, obtaining biomarker values that have been manually input, using an input device, or the like. Suitably, the indicator may be determined using a combination of a plurality of biomarker values, the indicator being at least partially indicative of the likelihood that a subject has ovarian cancer. Assuming the method is performed using an electronic processing device, an indication of the indicator is optionally displayed or otherwise provided to the user.

In some embodiments, biomarker values are combined, for example by adding, multiplying, subtracting, or dividing biomarker values to determine an indicator value. This step is performed so that multiple biomarker values can be combined into a single indicator value, providing a more useful and straightforward mechanism for allowing the indicator to be interpreted and hence used in determining the likelihood of a subject responding to cancer therapy.

It will be understood that in this context, the biomarkers used within the above-described method can define a biomarker profile for cancer therapy responsiveness, which includes a minimal number of biomarkers (e.g., at least one biomarker), whilst maintaining sufficient performance to allow the biomarker profile to be used in making a clinically relevant determination. Minimizing the number of biomarkers used minimizes the costs associated with performing diagnostic or prognostic tests and in the case of polypeptide biomarkers, allows the test to be performed utilizing relatively straightforward techniques such as immunoassay or lectin-immunoassay, and allowing the test to be performed rapidly in a clinical environment. In this regard, the indication provided by the methods described herein could be a graphical or alphanumeric representation of an indicator value. Alternatively, however, the indication could be the result of a comparison of the indicator value to predefined thresholds or ranges, or alternatively could be an indication of the likelihood of a subject having ovarian cancer.

Furthermore, producing a single indicator value allows the results of the test to be easily interpreted by a clinician or other medical practitioner, so that test can be used for reliable diagnosis in a clinical environment.

Solely by way of an illustration, the indicator-determining methods suitably include determining at least one biomarker value, wherein the biomarker value is a value measured or derived for at least one glycospecies of a glycoprotein and is at least partially indicative of a concentration or abundance of the glycospecies of the glycoprotein in a sample taken from the subject.

The derived biomarker value is then used to determine the indicator for use in determining the likelihood of a subject responding to cancer therapy, either by using the derived biomarker value as an indicator value, or by performing additional processing, such as comparing the derived biomarker value to a reference or the like, as generally known in the art and as described in more detail below, or to another biomarker value. In some embodiments, the indicator is indicative of a level, concentration or abundance of an expression product of AAL-A1AT. In other embodiments, the indicator is indicative of a level, concentration or abundance of AAL-AACT. In some embodiments, the indicator is indicative of a level, concentration or abundance of AAL-CO9. In some embodiments, the indicator is indicative of a level, abundance or concentration of AAL-HPT. In some embodiments, the indicator is indicative of to a level, abundance, or concentration of AAL-A2MG. In further embodiments, the indicator is indicative of a level or abundance ofSNA-A1AT. In some embodiments, the indicator is indicative of a level, concentration, or abundance of SNA-CO9. In some embodiments, the indicator is indicative of a level, abundance or concentration of SNA-ITIG3. In some embodiments, the indicator is indicative of a level, abundance or concentration of SNA-A2MG. In some embodiments, the indicator is indicative of a level, abundance or concentration of SNA-ALS. In some embodiments, the indicator is indicative of a level, abundance or concentration of SNA-IBP3. In some embodiments, the indicator is indicative of a level, abundance or concentration of STL-CO9. In some embodiments, the indicator is indicative of a level, abundance or concentration of STL-ITIH3. In some embodiments, the indicator is indicative of a level, abundance or concentration of STL-A2MG. In some embodiments, the indicator is indicative of a level, abundance or concentration of STL-ALS. In some embodiments, the indicator is indicative of a level, abundance or concentration of STL-IBP3. In some embodiments, the indicator is indicative of a level, abundance or concentration of STL-PON1. Furthermore, the invention may also include determining the presence or absence of mutations in tissue pathology.

The derived biomarker values could be combined using a combining function such as an additive model; a linear model; a support vector machine; a neural network model; a random forest model; a regression model; a genetic algorithm; an annealing algorithm; a weighted sum; a nearest neighbour model; and a probabilistic model. In some embodiments, the indicator is compared to an indicator reference, with a likelihood of responsiveness to cancer being determined in accordance with results of the comparison. The indicator reference may be derived from indicators determined for a number of individuals in a reference population. The reference population typically includes individuals having different characteristics, such as a plurality of individuals of different sexes; and/or ethnicities, with different groups being defined based on different characteristics, with the subject's indicator being compared to indicator references derived from individuals with similar characteristics. The reference population can include a plurality of individuals known to have ovarian cancer; or a plurality of individuals known to not to have ovarian cancer (i.e., healthy subjects).

In specific embodiments, the indicator-determining methods of the present invention are performed using at least one electronic processing device, such as a suitably programmed computer system or the like. In this case, the electronic processing device typically obtains at least one measured biomarker value, either by receiving this from a measuring or other quantifying device, or by retrieving these from a database or the like. The processing device then determines the indicator by any suitable means, for example, by calculating a value that is indicative of a ratio of concentrations or amounts of a first glycospecies of a glycoprotein and concentrations or amounts of a second glycospecies of the same or different glycoprotein. In one aspect, the present invention encompasses an apparatus comprising such electronic processing device(s).

The processing device can then generate a representation of the indicator, for example by generating a sign or alphanumeric indication of the indicator, a graphical indication of a comparison of the indicator to one or more indicator references or an alphanumeric indication of the likelihood that the subject has ovarian cancer.

The indicator-determining methods of the present invention typically include obtaining a sample from a subject who is suspected to have, or is at risk of having ovarian cancer, wherein the sample includes one or more glycospecies of a glycoprotein and quantifying or otherwise assessing at least one of the biomarkers within the sample to determine biomarker values. This can be achieved using any suitable technique, and will depend on the nature of the biomarker, as described above. Suitably, an individual measured or biomarker value corresponds to the level, abundance or concentration of a glycospecies of a glycoprotein. For example, if the indicator in some embodiments of the indicator-determining method of the present invention, which uses a plurality of glycospecies, is based on a ratio of concentrations of two glycospecies of a glycoprotein. This process would typically include quantifying the glycospecies by any means known in the art, including measuring binding of the glycospecies to a glycan-binding agent.

In some embodiments, the likelihood of a subject having ovarian cancer is established by determining one or more glycospecies values, wherein a glycospecies value is indicative of a value measured or derived for a glycospecies of a glycoprotein in a subject or in a sample obtained from the subject. These biomarkers are referred to herein as “sample glycospecies of a glycoprotein.” In accordance with the present invention, a sample glycospecies of a glycoprotein will correspond to a reference glycospecies of a glycoprotein (also referred to herein as a “corresponding glycospecies of a glycoprotein”).

### 3. Biomarker Detection Kits, Compositions and Supports

All the essential reagents required for detecting and quantifying the ovarian cancer biomarkers of the invention may be assembled together in a kit. In some embodiments, the kit comprises a reagent that permits quantification of at least one glycospecies of a glycoprotein. In some embodiments, the kit comprises: (i) at least one reagent that allows quantification (e.g., determining the abundance, concentration or level) of a glycospecies of a glycoprotein in a biological sample; and optionally (ii) instructions for using the at least one reagent. In some embodiments, the kit further comprises (iii) at least one reagent that allows quantification of the total amount of the glycoprotein in the biological sample; and/or (iv) at least one reagent that allows quantification of a different glycospecies of the glycoprotein in the biological sample.

In the context of the present invention, “kit” is understood to mean a product containing the different reagents necessary for carrying out the methods of the invention packed so as to allow their transport and storage. Materials suitable for packing the components of the kit include crystal, plastic (polyethylene, polypropylene, polycarbonate and the like), bottles, vials, paper, envelopes and the like. Additionally, the kits of the invention can contain instructions for the simultaneous, sequential or separate use of the different components contained in the kit. The instructions can be in the form of printed material or in the form of an electronic support capable of storing instructions such that they can be read by a subject, such as electronic storage media (magnetic disks, tapes and the like), optical media (CD-ROM, DVD) and the like. Alternatively, or in addition, the media can contain internet addresses that provide the instructions.

Reagents that allow quantification of a glycospecies include compounds or materials, or sets of compounds or materials, which allow quantification of the glycospecies. In specific embodiments, the compounds, materials or sets of compounds or materials permit determining the binding to a glycan-binding agent.

The kits may also optionally include appropriate reagents for internal standards, positive and negative controls, washing solutions, blotting membranes, microtiter plates, dilution buffers and the like. For example, a protein-based detection kit may include (i) at least one glycospecies of a glycoprotein; and (ii) a glycan-binding agent that binds specifically to the glycospecies. In some embodiments the glycan-binding agent includes a lectin. Lectins can be pre-conjugated on a support substrate such as magnetic bead, or nanoparticles with surface-enhanced Raman spectroscopy (SERS) barcodes. In some of the same embodiments and some alternative embodiments lectins could be pre-coupled to fluorescent molecules for detection.

The kit can also feature various devices (e.g., one or more) and reagents (e.g., one or more) for performing one of the assays described herein; and/or printed instructions for using the kit to quantify the level or abundance of a glycospecies of the glycoprotein.

Also provided may be compositions and/or solid supports for determining an indicator used in assessing a likelihood of a subject having ovarian cancer.

### 4. Therapeutic Applications

The present invention also extends to the management and treatment of subjects with ovarian cancer. For example, where the methods of the present invention are used to detect the presence of ovarian cancer in a subject, the methods can further comprise informing treatment selection for the ovarian cancer based on the biomarker profile. Therapies for ovarian cancer are well known in the art and an appropriate therapeutic regimen for a particular subject, based on the severity of the disease and other factors, such as age and general health of the subject, can be determined by a skilled practitioner and administered appropriately without undue experimentation.

Treatment options for ovarian cancer can vary depending on the stage of the cancer, i.e, stage 1, 2, 3 or 4, and can include surgery to remove the ovaries, fallopian tubes (oophorectomy) or uterus (hysterectomy) that contain the cancer, chemotherapy, immunotherapies and/or radiation therapy.

Radiotherapies include radiation and waves that induce DNA damage for example, γ-irradiation, X-rays, UV irradiation, microwaves, electronic emissions, radioisotopes, and the like. Therapy may be achieved by irradiating the localised tumour site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Non-limiting examples of radiotherapies include conformal external beam radiotherapy (50-100 Grey given as fractions over 4-8 weeks), either single shot or fractionated, high dose rate brachytherapy, permanent interstitial brachytherapy, systemic radio-isotopes (e.g., Strontium 89). In some embodiments the radiotherapy may be administered in combination with a radiosensitizing agent. Illustrative examples of radiosensitizing agents include but are not limited to efaproxiral, etanidazole, fluosol, misonidazole, nimorazole, temoporfin and tirapazamine.

Chemotherapeutic agents may be selected from any one or more of the following categories:
(i) antiproliferative,/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas), antimetabolites (for example antifolates such as fluoropyridines like 5-fluorouracil and tegafur, raltitrexed, methotrexate, cytosine arabinoside and hydroxyurea, anti-tumour antibiotics (for example anthracyclines like adriamycin, bleomycin, doxorubicin, daunomycin, epirubiein, idarubicin, mitomycin-C, daetinomycin and mitliramycin), antimitotic agents (for example vinea alkaloids like vincristine, vinblastine, vindesine and vinorelbine and taxoids like paclitaxel and docetaxel, and topoisomefase inhibitors (for example epipodophyllotoxins like etoposide and teniposide, amsaerine, topotecan and camptothecin);
(ii) cytostatic agents such as antiestrogens (for example tamoxifen, toremifene, raloxifene, droloxiiene and idoxifene), oestrogen receptor down regulators (for example furvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorozole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy) quinazolin-4-amine (gefitinib, AZD1839), N-(3-ethynylphenyl)-6,7-bis(2-methoxyethoxy) quinazolin-4-amine (erlotinib, OSI-774) and 6-actylamido-N-(3-chloro-4-fiuorophenyl)-7-(3-morpholinopropoxy) quinazoli-n-4-amine (CI 1033)), for example inhibitors of the platelet-derived growth factor family and for example inhibitors of the hepatocyte growth factor family;
(v) anti-angiogenic agents such as those which inhibit the effects of vascular endothelial growth, factor, (for example the anti-vascular endothelial cell growth factor antibody bevacizumab [Avastin™], compounds such as those disclosed in International Patent Publication Nos. WO 97/22596, WO 97/30035, WO 97/32856 and WO 98/13354) and compounds that work by other mechanisms (for example linomide, inhibitors of integrin avβ3 function and angiostatin);
(vi) vascular damaging agents such as Combretastatin A4 and compounds disclosed in International Patent Publication Nos. WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense, and
(viii) gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Immunotherapy approaches, include for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

Examples of other cancer therapies include phototherapy, cryotherapy, toxin therapy or pro-apoptosis therapy. One of skill in the art would know that this list is not exhaustive of the types of treatment modalities available for cancer and other hyperplastic lesions.

In instances where the cancer is HER2-positive, treatment may also include administration of an anti-HER2 antibody, such as trastuzumab.

Where the methods of the present invention are used to monitor the progress of ovarian cancer in a subject that has undergone or is undergoing treatment, and/or assess the efficacy of treatment, the methods may also include modifying or altering the treatment. For example, if the level or ratio of one or more glycospecies identified herein indicates that the disease has progressed and the current or previous treatment protocol has been ineffective, a skilled practitioner may devise a modified or altered treatment protocol. For example, if the subject has undergone surgery to remove the ovaries, and the level or ratio of one or more glycospecies identified herein indicates that the disease has progressed post-surgery, the subject may be administered chemotherapy and/or radiotherapy. Conversely, if the level or ratio of one or more glycospecies identified herein indicates that the disease has regressed and the current or previous treatment protocol has been effective, a skilled practitioner may continue the current or previous treatment protocol to continue regression of the disease, or may choose to reduce or discontinue the current or previous treatment protocol. Further, the methods of the present invention can also be used to determine the likelihood of a subject who has undergone a treatment regimen (e.g., surgery) having a relapse of ovarian cancer. Preferably, the subject would be monitored at a time after the treatment regimen (e.g., after 1 day, 2 days, 3 days, 4 days, 5 days, 6 days, 1 week, 2 weeks, 3 weeks, 1 month, 2 months, 3 months, 4 months 6 months or more than 6 months) to determine the likelihood of the subject having a relapse of ovarian cancer.

Typically, therapeutic agents as described for example above will be administered in pharmaceutical compositions together with a pharmaceutically acceptable carrier and in an effective amount to achieve their intended purpose. The dose of active compounds administered to a subject should be sufficient to achieve a beneficial response in the subject over time such as a reduction in, or relief from, the symptoms of ovarian cancer. The quantity of the pharmaceutically active compounds(s) to be administered may depend on the subject to be treated inclusive of the age, sex, weight and general health condition thereof. In this regard, precise amounts of the active compound(s) for administration will depend on the judgment of the practitioner.

### 5. Methods of Monitoring Treatment

The present invention can be practiced in the field of predictive medicine for the purposes of diagnosis or monitoring the presence or development of a condition such as ovarian cancer in a subject, and/or monitoring response to therapy efficacy.

The glycospecies profiles of the present invention further enable determination of endpoints in pharmacotranslational studies. For example, clinical trials can take many months or even years to establish the pharmacological parameters for a medicament to be used in treating or preventing ovarian cancer. However, these parameters may be associated with a glycospecies profile associated with a health state (e.g., healthy control). Hence, the clinical trial can be expedited by selecting a treatment regimen medicament and pharmaceutical parameters, which results in a glycospecies profile associated with the desired health state (e.g., healthy control). This may be determined for example by (1) providing a correlation of a reference glycospecies profile with the likelihood of having healthy control, (2) obtaining a corresponding glycospecies profile of a subject having ovarian cancer, after treatment with a treatment regimen, wherein a similarity of the subject's glycospecies profile after treatment to the reference glycospecies profile indicates the likelihood that the treatment regimen is effective for changing the health status of the subject to the desired health state (e.g, healthy control). This aspect of the present invention advantageously provides methods of monitoring the efficacy of a particular treatment regimen in a subject (for example, in the context of a clinical trial) already diagnosed with a condition selected from ovarian cancer.

These methods take advantage of glycospecies biomarkers that correlate with treatment efficacy, for example, to determine whether the glycospecies profile of a subject undergoing treatment partially or completely normalizes during the course of or following therapy or otherwise shows changes associated with responsiveness to the therapy.

The glycospecies profiles further enable stratification of patients prior to enrolment in pharmacotranslational studies. For example, a clinical trial can be expedited by selecting a priori patients with a particular glycospecies profile that would most benefit from a particular treatment regimen (e.g., medicament and pharmaceutical parameters). For instance, patient enrolment into a clinical trial testing the efficacy of a new ovarian cancer therapeutic would best include patients with a glycospecies profile that indicated that they had ovarian cancer rather than benign cancer and as such the selected patients would most likely benefit from the new therapy.

Thus, the invention provides methods of correlating a reference glycospecies profile with an effective treatment regimen for a condition selected from ovarian cancer, wherein the reference glycospecies profile evaluates at least one (e.g., 1, 2, 3, 4, 5, 6, 7, 8, 9, 10, etc.) glycospecies biomarker. These methods generally comprise: (a) determining a sample glycospecies profile from a subject with the condition prior to treatment (i.e., baseline), wherein the sample glycospecies profile evaluates for an individual glycospecies biomarker in the reference glycospecies profile a corresponding glycospecies biomarker, and correlating the sample glycospecies profile with a treatment regimen that is effective for treating that condition.

The invention further provides methods of determining whether a treatment regimen is effective for treating a subject with ovarian cancer. These methods generally comprise: (a) correlating a reference glycospecies profile prior to treatment (i.e., baseline) with an effective treatment regimen for the condition, wherein the reference glycospecies profile evaluates at least one (e.g., 1, 2, 3, 4, 5, 6, 7, 8, 9, 10, etc) glycospecies biomarker, and (b) obtaining a sample glycospecies profile from the subject after treatment, wherein the sample glycospecies profile evaluates for an individual glycospecies biomarker in the reference glycospecies profile a corresponding glycospecies biomarker, and wherein the sample glycospecies profile after treatment indicates whether the treatment regimen is effective for treating the condition in the subject.

The invention can also be practiced to evaluate whether a subject is responder (i.e., a positive response) or non-responder (i.e., no response) to a treatment regimen. This aspect of the invention provides methods of correlating a glycospecies profile with a positive and/or negative response to a treatment regimen. These methods generally comprise: (a) obtaining a glycospecies profile from a subject with a condition selected from ovarian cancer, following commencement of the treatment regimen, wherein the glycospecies profile evaluates at least one (e.g., 1, 2, 3, 4, 5, 6, 7, 8, 9, 1.0, etc.) glycospecies biomarker, and (b) correlating the glycospecies from the subject with a positive and/or negative response to the treatment regimen.

The invention also provides methods of determining a positive and/or negative response to a treatment regimen by a subject with ovarian cancer. These methods generally comprise: (a) correlating a reference glycospecies profile with a positive and/or negative response to the treatment regimen, wherein the reference glycospecies profile evaluates at least one (e.g., 1, 2, 3, 4, 5, 6, 7, 8, 9, 10, etc.) glycospecies biomarker, and (b) determining a sample glycospecies profile from the subject, wherein the subject's sample glycospecies profile evaluates for an individual glycospecies biomarker in the reference glycospecies profile a corresponding glycospecies biomarker and indicates whether the subject is responding to the treatment regimen.

In some embodiments, the methods further comprise determining a first sample glycospecies profile from the subject prior to commencing the treatment regimen (i.e., a baseline profile), wherein the first sample glycospecies profile evaluates at least one (e.g., 3, 2, 3, 4, 5, 6, 7, 8, 9, 10, etc.) glycospecies biomarker, and comparing the first sample glycospecies profile with a second sample glycospecies profile from the subject after commencement of the treatment regimen, wherein the second sample glycospecies profile evaluates for an individual glycospecies biomarker in the first sample glycospecies profile a corresponding glycospecies biomarker. This aspect of the invention can be practiced to identify responders or non-responders relatively early in the treatment process, i.e., before clinical, manifestations of efficacy. In this way, the treatment regimen can optionally be discontinued, a different treatment protocol can be implemented and/or supplemental therapy can be administered. Thus, in some embodiments, a sample glycospecies profile is obtained within about 2 hours, 4 hours, 6 hours, 32 hours, 1 day, 2 days, 3 days, 4 days, 5 days, 1 week, 2 weeks, 3 weeks, 4 weeks, 6 weeks, 8 weeks, 10 weeks, 12 weeks, 4 months, six months or longer of commencing therapy.

In order that the invention may be readily understood and put into practical effect, particular preferred embodiments will now be described by way of the following non-limiting examples.

### EXAMPLES

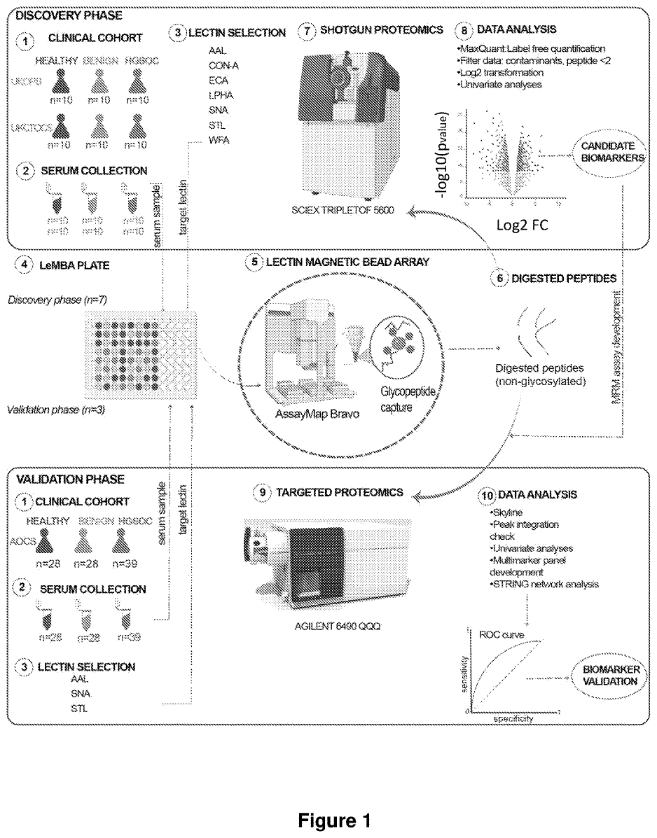

To discover and validate glycoprotein biomarkers for ovarian cancer, the inventors undertook case-control studies using serum samples from women with HGSOC patients (cases) and two age-matched groups (controls)—women with a benign ovarian neoplasm and healthy women. Lectin magnetic bead array-coupled mass spectrometry (LeMBA-MS) technology in two study phases, using three independent cohorts of serum samples as illustrated in FIG. 1.

The discovery phase analysed two cohorts, (1) a clinical set of 30 serum samples from the United Kingdom Ovarian Population Study (UKOPS) collected from women at diagnosis of HGSOC (n=10), benign ovarian neoplasms (n=10) and healthy controls (n=10), and (2) a pre-clinical set of 30 serum samples from the UKCTOCS (United Kingdom Collaborative Trial of Ovarian Cancer Screening) trial (Menon et al.) collected from women at a mean interval of 11.1±5.1 months prior to diagnosis of HGSOC (n=10), benign ovarian neoplasms (n=10) and healthy controls (n=10). Discovery phase data were analysed for glycoprotein candidates that are differentially abundant between ovarian cancer group and healthy or benign groups, to generate a list of biomarker candidates and a multiple reaction monitoring mass spectrometry (MRM-MS) method to measure proteotypic peptides from candidate glycoproteins in the validation phase. The candidate glycoproteins are indicated by the nomenclature “lectin-UniProt entry name”. The validation phase analysed a clinical set of 95 serum samples from the Australian Ovarian Cancer Study (AOCS) collected from women at diagnosis of HGSOC (n=39), benign ovarian neoplasms (n=28) and healthy controls (n=28).

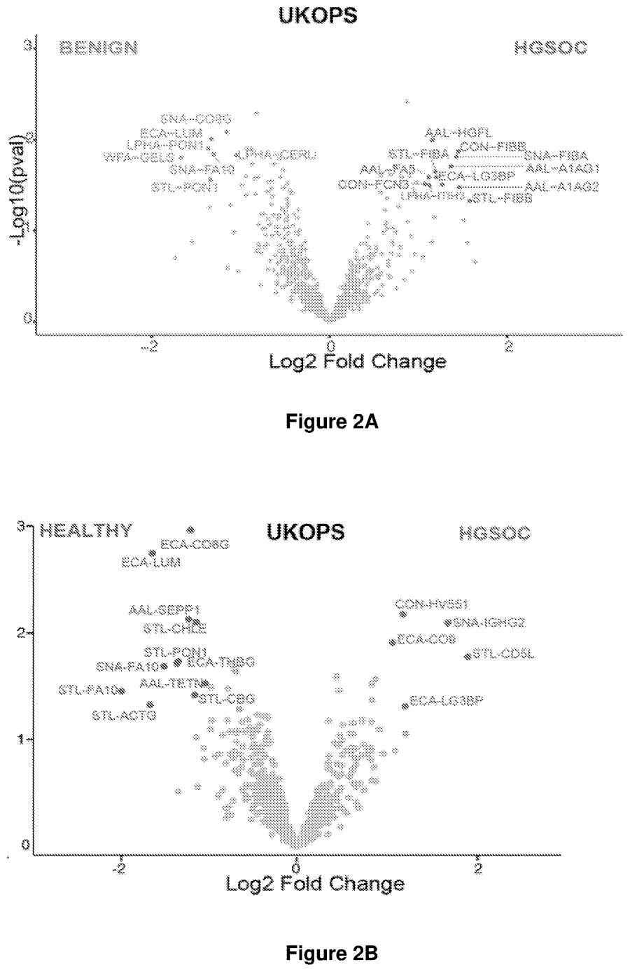

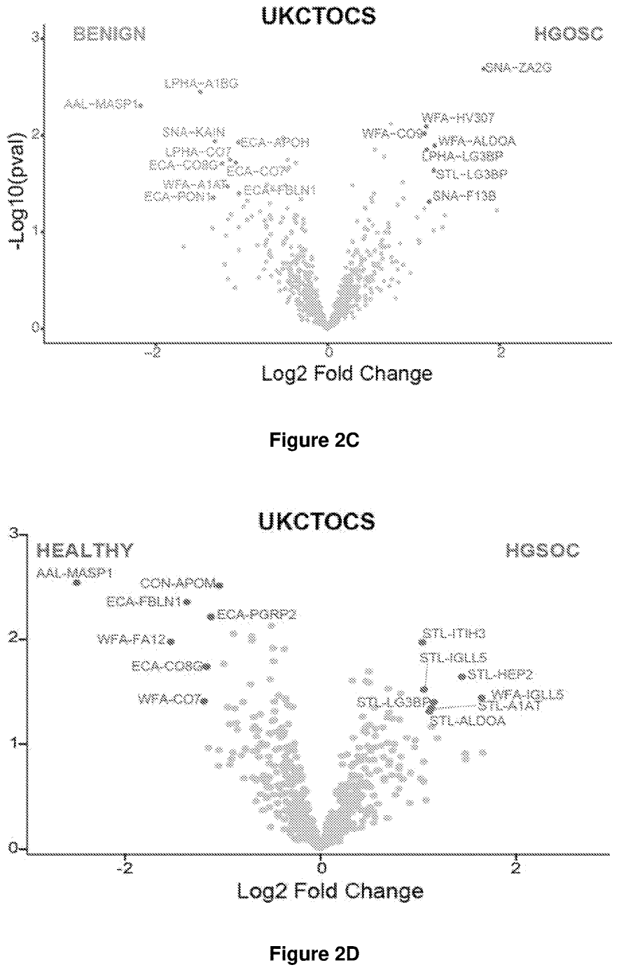

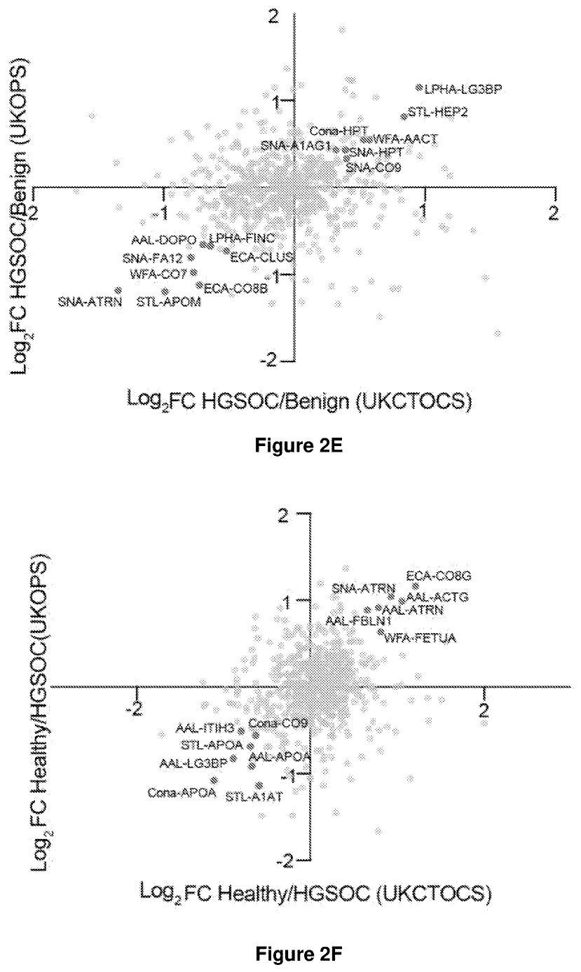

In the UKOPS cohort the inventors found 15 and 16 differentially abundant proteins (p value <0.05) in ovarian cancer samples when compared to benign and healthy samples respectively (FIG. 2A, Table 3). The ovarian cancer samples compared to benign samples, exhibited an increase in AAL lectin associated proteins such as alpha-1-acid-glycoprotein 1 (A1AG1), alpha-1-acid-glycoprotein-2 (A1AG2), coagulation factor V (FA5) and hepatocyte growth factor (HGF2), STL lectin associated fibrinogen alpha (FIBA) and beta chain (FIBB) proteins and LPHA lectin associated inter-alpha-trypsin-inhibitor heavy chain 3 (ITIH3) protein. While a corresponding reduction in SNA lectin associated complement component C8 gamma chain (CO8G) and coagulation factor X (FA10) proteins, and LPHA and STL lectin associated serum paraoxonase/arylesterase 1 (PON1) protein was observed for these ovarian cancer samples compared to the benign samples (FIG. 2A). In contrast when compared to the healthy samples, the UKOPS ovarian cancer samples exhibited an increase in ECA lectin associated proteins including complement component C9 (CO9) and galectin-3-binding protein (LG3BP) (FIG. 2B). While a corresponding reduction of ECA lectin associated proteins, such as, complement component C8 beta (CO8B) and gamma chains (CO8G), lumican (LUM) and thyroxine-binding globulin (THBG), along with STL lectin associated actin (ACTG), corticosteroid-binding globulin (CBG), cholinesterase (CHLE) and PON1 proteins was observed for these ovarian cancer samples when compared to healthy samples (FIG. 2B).

TABLE 3
DISCOVERY PHASE BIOMARKER CANDIDATES

| | HGSOC vs Benign | | HGSOC vs Healthy | | TOTAL |
|---|---|---|---|---|---|
| LECTIN | UKOPS | UKCTOCS | UKOPS | UKCTOCS | NUMBER |
| AAL | A1AG1, | MASP1 | SEPP1 | MASP1 | 6 |
| | A1AG2, FA5, | | | | |
| | HGFL | | | | |
| CON-A | FIBB, FCN3 | — | HV551 | APOM | 4 |
| ECA | LUM, LG3BP | APOH, CO7, | CO9, LG3BP, | CO8G, FBLN1, | 11 |
| | | CO8G, FBLN1, | CO8B, CO8G, | PGRP2 | |
| | | PON1 | LUM, THBG | | |
| L-PHA | PON1, CERU, | — | — | — | 6 |
| | ITIH3 | | | | |
| SNA | FA10, CO8G, | KAIN, F13B, | IGHG2, CBPN, | — | 9 |
| | FIBA | ZA2G | FA10 | | |
| STL | PON1, FIBA, | LG3BP | CD5L, CHLE, | A1AT, ITIH3, | 13 |
| | FIBB | | PON1, FA10, | HEP2, IGLL5, | |
| | | | ACTG, CBG | LG3BP | |
| WFA | GELS | A1AT, CO9, | — | IGLL5, CO7, | 8 |
| | | ALDOA, HV307 | | FA12 | |
| TOTAL | 15 | 15 | 14 | 12 | |
| NUMBER | | | | | |

For each lectin, significant proteins with p-value < 0.05 and log₂FC > 1 identified in each clinical cohort have been detailed. The total number of candidate proteins across each lectin and clinical cohort accounts for overlaps. The proteins are labelled by their UniProt entry name.

In the UKCTOCS cohort, the inventors found 14 and 12 differentially abundant proteins (p value <0.05) in ovarian cancer samples when compared to benign and healthy samples respectively (Table 3). An increase in WFA lectin associated proteins such as fructose bisphosphates aldolase A (ALDOA), CO9 and immunoglobulin heavy-variable 3-7 (HV307), and SNA lectin associated proteins such as coagulation factor XIII B chain (F13B) and zinc-alpha-2-glycoprotein (ZA2G) in the ovarian cancer samples was also observed when compared to benign samples (FIG. 2C). These ovarian cancer samples also exhibited a significant reduction in the AAL lectin associated mannan-binding lectin serine protease 1 (MASP1) protein and ECA lectin associated proteins such as beta-2 glycoprotein-1 (APOH), complement component C7 (CO7), complement component C8 gamma chain (CO8G), fibulin-1 (FBLN1) and PON1 (FIG. 2C). In contrast, an increase in STL lectin associated proteins such as, alpha-1-antitrypsin (A1AT), heparin cofactor 2 (HEP2), ITIH3, immunoglobulin lambda like growth factor (IGLL5) and LG3BP, was seen in the ovarian cancer samples when compared to healthy samples (FIG. 2D). Additionally, these ovarian cancer samples also exhibited a significant reduction in AAL lectin associated MASP1 protein, ECA lectin associated CO8G, FBLN1 and peptidoglycan recognition protein-2 (PGRP2) proteins and WFA lectin associated CO7 and coagulation factor XII (FA12) proteins (FIG. 2D).

### Generation of Candidate List for Validation.

The list of protein candidates discovered from UKOPS and UKCTOCS were combined to generate a list of 44 proteins, which fell short of the target number of ~60 candidate proteins that the inventors previously used for biomarker validation (Shah, 2018; and Shah, 2015). In order to assess additional candidates which may be just outside of the p<0.05 cut-off, the inventors expanded the selection threshold to p-value <0.1 and removed Log₂ fold change filtering. This resulted in an initial list of 102 proteins which was filtered down to a final MRM target list of 59 candidate based on suitability of protein tryptic peptides for MRM. The developed custom MRM assay measured 170 peptides from the 59 candidate proteins, with at least 3 peptides per protein and 4-5 transitions per peptide.

The three lectins with the highest number of candidates in both UKOPS and UKCTOCS cohorts (AAL, SNA and STL) were selected for LeMBA-MRM-MS analyses on an independent validation cohort from AOCS, comprised of 95 age-matched donors. Univariate analysis for ovarian cancer vs benign and ovarian cancer vs healthy was conducted on each lectin dataset using adjusted p-value <0.05 to assess significance (TABLE 4 and TABLE 5).

TABLE 4
PEPTIDES SIGNIFICANTLY OVEREXPRESSED IN OVARIAN CANCER IN VALIDATION COHORT

| LECTIN | GLYCOPROTEIN | PEPTIDE | SEQ ID NO: |
|---|---|---|---|
| AAL | A1AG1 | EQLGEFYEALDCLR | 1 |
| | | TEDTIFLR | 2 |
| | | SDVVYTDWK | 3 |
| | | WFYIASAFR | 4 |
| | | EQLGEFYEALDCLCIPR | 5 |
| | A1AT | QINDYVEK | 6 |
| | | SVLGQLGITK | 7 |
| | | SASLHLPK | 8 |
| | | LLELTGPK | 9 |
| | A1BG | HQFLLTGDTQGR | 10 |
| | | ATWSGAVLAGR | 11 |
| | AACT | ADLSGITGAR | 12 |
| | | EQLSLLDR | 13 |
| | | ITLLSALVETR | 14 |
| | CERU | GAYPLSIEPIGVR | 15 |
| | CFAB | YGLVTYATYPK | 16 |
| | | EELLPAQDIK | 17 |
| | CFAI | GLETSLAECTFTK | 18 |
| | CO2 | EILNINQK | 19 |
| | | RNDYLDIYAIGVGK | 20 |
| | | AVISPGFDVFAK | 21 |
| | CO9 | VVEESELAR | 22 |
| | | LSPIYNLVPVK | 23 |
| | | FTPTETNK | 24 |
| | FA5 | LLSLGAGEFK | 25 |
| | FA10 | ETYDFDIAVLR | 26 |
| | FIBA | VQHIQLLQK | 27 |
| | FIBB | SILENLR | 28 |
| | | QDGSVDFGR | 29 |
| | HPT | DYAEVGR | 30 |
| | | VTSIQDWVQK | 31 |
| | HPTR | NYAEVGR | 32 |
| | IC1 | TLYSSSPR | 33 |
| | ITIH3 | ALDLSLK | 34 |
| | | DYIFGNYIER | 35 |
| | ITIH4 | LGVYELLLK | 36 |
| | | SPEQQETVLDGNLIIR | 37 |
| | | ILDDLSPR | 38 |
| | PEDF | LAAAVSNFGYDLYR | 39 |
| | VTDB | VLEPTLK | 40 |
| | | RTHLPEVFLSK | 41 |
| SNA | A1AG1 | EQLGEFYEALDCLR | 1 |
| | | SDVVYTDWK | 3 |
| | A1AT | QINDYVEK | 6 |
| | | SVLGQLGITK | 7 |
| | | SASLHLPK | 8 |
| | AACT | ADLSGITGAR | 12 |
| | | EQLSLLDR | 13 |
| | | ITLLSALVETR | 14 |
| | CFAB | YGLVTYATYPK | 16 |
| | CO9 | VVEESELAR | 22 |
| | | LSPIYNLVPVK | 23 |
| | | FTPTETNK | 24 |
| | FA10 | ETYDFDIAVLR | 26 |
| | FIBA | VQHIQLLQK | 27 |
| | | LKNSLFEYQK | 42 |
| | FIBB | SILENLR | 28 |
| | | QDGSVDFGR | 29 |
| | HPT | DYAEVGR | 30 |
| | | VTSIQDWVQK | 31 |
| | HPTR | NYAEVGR | 32 |
| | | YVMLPVADQYDCITHYEGSTCPK | 43 |
| | | VVLHPNYHQVDIGLIK | 44 |
| | IC1 | TLYSSSPR | 33 |
| | ITIH3 | ALDLSLK | 34 |
| | | EHLVQATPENLQEAR | 45 |
| | CO9 | VVEESELAR | 22 |
| | | LSPIYNLVPVK | 23 |
| | | FTPTETNK | 24 |
| | FIBA | VQHIQLLQK | 27 |
| | FIBB | SILENLR | 28 |
| | | QDGSVDFGR | 29 |
| | HPT | VTSIQDWVQK | 30 |
| | | DYAEVGR | 31 |
| | HPTR | NYAEVGR | 32 |
| | LUM | ISNIPDEYFK | 46 |

TABLE 5
PEPTIDES SIGNIFICANTLY UNDEREXPRESSED IN OVARIAN CANCER IN VALIDATION COHORT

| LECTIN | GLYCOPROTEIN | PEPTIDE | SEQ ID NO: |
|---|---|---|---|
| AAL | A2MG | NEDSLVFVQTDK | 47 |
| | | QGIPFFGQVR | 48 |
| | | SIYKPGQTVK | 49 |
| | AMBP | QINDYVEK | 50 |
| | C1QC | TNQVNSGGVLLR | 51 |
| | | QTHQPPAPNSLIR | 52 |
| | CBG | ADLSGITGAR | 53 |
| | CHLE | YLTLNTESTR | 54 |
| | FETUA | HTLNQIDEVK | 55 |
| | | CNLLAEK | 56 |
| | | FSVVYAK | 57 |
| | FIBB | YQISVNK | 58 |
| | GELS | QTQVSVLPEGGETPLFK | 59 |
| | IBP3 | ALAQCAPPPAVCAELVR | 60 |
| | | FLNVLSPR | 61 |
| | ITIH1 | VTYDVSR | 62 |
| | | LDAQASFLPK | 63 |
| | ITIH2 | IYLQPGR | 64 |
| | | NDLISATK | 65 |
| | | VQSTITSR | 66 |
| | PON1 | SFNPNSPGK | 67 |
| | | STVELFK | 68 |
| | | IFFYDSENPPASEVLR | 69 |
| | TRFE | EFQLFSSPHGK | 70 |
| | | YLGEEYVK | 71 |
| | | DDTVCLAK | 72 |
| | | RTHLPEVFLSK | 73 |
| SNA | A1AG2 | EHVAHLLFLR | 74 |
| | A2AP | NKFDPSLTQR | 75 |
| | | LFGPDLK | 76 |
| | | LGNQEPGGQTALK | 77 |
| | A2MG | FEVQVTVPK | 78 |
| | | SIYKPGQTVK | 49 |
| | | QGIPFFGQVR | 48 |
| | ALS | LAELPADALGPLQR | 79 |
| | | LEALPNSLLAPLGR | 80 |
| | AMBP | TVAACNLPIVR | 81 |
| | | AFIQLWAFDAVK | 82 |
| | ANGT | SLDFTELDVAAEK | 83 |
| | | DPTFIPAPIQAK | 84 |
| | C1QC | QTHQPPAPNSLIR | 52 |
| | | FQSVFTVTR | 85 |
| | | TNQVNSGGVLLR | 51 |
| | CBG | ITQDAQLK | 86 |
| | | QINSYVK | 87 |
| | CHLE | NIAAFGGNPK | 88 |
| | | YLTLNTESTR | 54 |
| | CO6 | QLEWGLER | 89 |
| | CO7 | VLFYVDSEK | 90 |
| | | LTPLYELVK | 91 |
| | CPN2 | GQVVPALNEK | 92 |
| | | QLVCPVTR | 93 |
| | | LELLSLSK | 94 |
| | FA10 | IVGGQECK | 95 |
| | FA12 | VVGGLVALR | 96 |
| | | EQPPSLTR | 97 |
| | | CFEPQLLR | 98 |
| | FETUA | HTLNQIDEVK | 55 |
| | | FSVVYAK | 57 |
| | | CNLLAEK | 56 |
| | FIBBB | YQISVNK | 58 |
| | GELS | QTQVSVLPEGGETPLFK | 59 |
| | HEMO | EVGTPHGIILDSVDAAFICPGSSR | 99 |
| | | NFPSPVDAAFR | 100 |
| | | GGYTLVSGYPK | 101 |
| | HEP2 | LNILNAK | 102 |
| | | YEITTIHNLFR | 103 |
| | | SVNDLYIQK | 104 |
| | | NYNLVESLK | 105 |
| | IBP3 | ALAQCAPPPAVCAELVR | 60 |
| | | FLNVLSPR | 61 |
| | | YGQPLPGYTTK | 106 |
| | ITIH1 | LDAQASFLPK | 63 |
| | | VTYDVSR | 62 |
| | | EVAFDLEIPK | 107 |
| | ITIH2 | VQSTITSR | 66 |
| | | NDLISATK | 65 |
| | | IYLQPGR | 64 |
| | KNG1 | EGDCPVQSGK | 108 |
| | | ENFLFLTPDCK | 109 |
| | | QVVAGLNFR | 110 |
| | PLMN | EAQLPVIENK | 111 |
| | | WELCDIPR | 112 |
| | PON1 | STVELFK | 68 |
| | | QTHQPPAPNSLIR | 113 |
| | PGRP2 | DTLPSCAVR | 114 |
| | | AGLLRPDYALLGHR | 115 |
| | THRB | ETAASLLQAGYK | 116 |
| | TRFE | EFQLFSSPHGK | 70 |
| | | YLGEEYVK | 71 |
| | | DDTVCLAK | 72 |
| STL | A2MG | FEVQVTVPK | 78 |
| | | NEDSLVFVQTDK | 47 |
| | | SIYKPGQTVK | 49 |
| | | QGIPFFGQVR | 48 |
| | A2AP | NKFDPSLTQR | 75 |
| | | LFGPDLK | 76 |
| | | LGNQEPGGQTALK | 77 |
| | ALS | LAELPADALGPLQR | 79 |
| | | LEALPNSLLAPLGR | 80 |
| | AMBP | TVAACNLPIVR | 81 |
| | | AFIQLWAFDAVK | 82 |
| | ANGT | QPFVQGLALYTPVVLPR | 117 |
| | C1QC | QTHQPPAPNSLIR | 52 |
| | | FQSVFTVTR | 85 |
| | | TNQVNSGGVLLR | 51 |
| | CBG | ITQDAQLK | 86 |
| | | QINSYVK | 87 |
| | CHLE | NIAAFGGNPK | 88 |
| | | YLTLNTESTR | 54 |
| | CPN2 | GQVVPALNEK | 92 |
| | | QLVCPVTR | 93 |
| | | LELLSLSK | 94 |
| | FA10 | IVGGQECK | 95 |
| | FA12 | VVGGLVALR | 96 |
| | | EQPPSLTR | 97 |
| | FETUA | HTLNQIDEVK | 55 |
| | | FSVVYAK | 57 |
| | | CNLLAEK | 56 |
| | FIBBB | YQISVNK | 58 |
| | GELS | QTQVSVLPEGGETPLFK | 59 |
| | HEP2 | LNILNAK | 102 |
| | | SVNDLYIQK | 104 |
| | HPTR | YVMLPVADQYDCITHYEGSTCPK | 118 |
| | | VVLHPNYHQVDIGLIK | 119 |
| | IBP3 | ALAQCAPPPAVCAELVR | 60 |
| | | FLNVLSPR | 61 |
| | | YGQPLPGYTTK | 106 |
| | ITIH1 | LDAQASFLPK | 63 |
| | | VTYDVSR | 62 |
| | | EVAFDLEIPK | 107 |
| | ITIH2 | VQSTITSR | 66 |
| | | NDLISATK | 65 |
| | | IYLQPGR | 64 |
| | ITIH4 | ILDDLSPR | 120 |
| | | LGVYELLLK | 121 |
| | LG3BP | SQLVYQSR | 122 |
| | KNG1 | EGDCPVQSGK | 108 |
| | | ENFLFLTPDCK | 109 |
| | PLMN | NLDENYCR | 123 |
| | | EAQLPVIENK | 111 |
| | | WELCDIPR | 112 |
| | PON1 | STVELFK | 68 |
| | | SFNPNSPGK | 67 |
| | | IFFYDSENPPASEVLR | 69 |
| | PGRP2 | DTLPSCAVR | 114 |
| | TRFE | EFQLFSSPHGK | 70 |
| | | YLGEEYVK | 71 |
| | | DDTVCLAK | 72 |

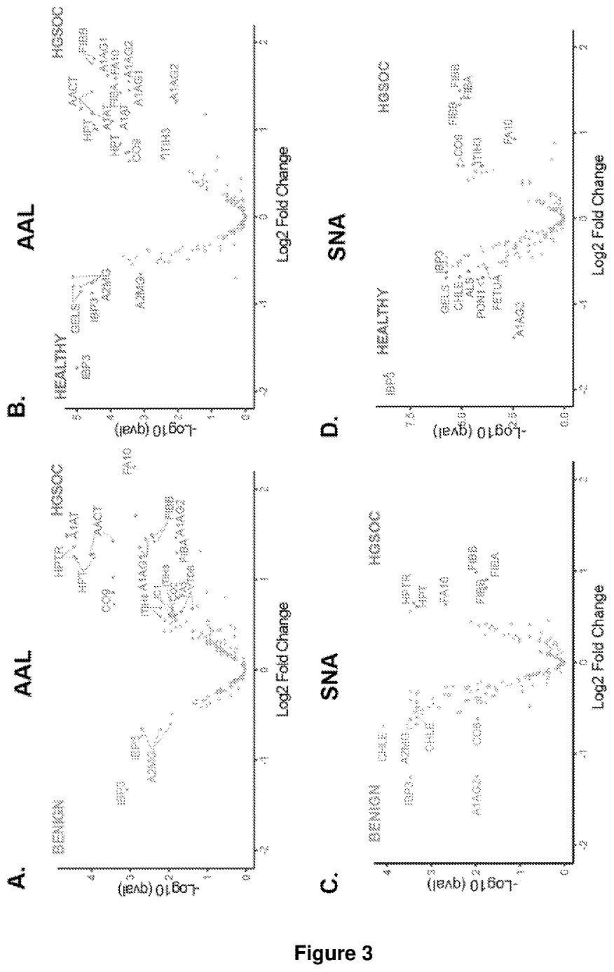

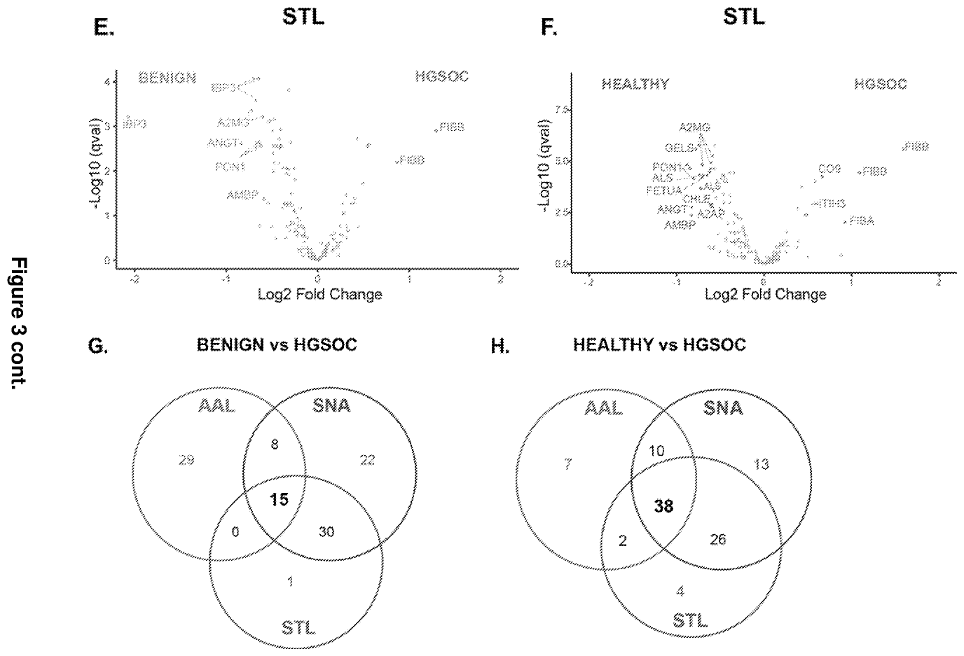

Volcano plots were used for visualisation with cut-offs set at adjusted p-value <0.05 and log 2 fold change >0.5 (FIG. 3). In this analysis, for the ovarian cancer vs benign ovarian neoplasm comparison, the inventors found 53 (AAL), 80 (SNA) and 49 (STL) peptides with an overlap of 15 common peptides that were mapped to 7 proteins. Likewise, for the ovarian cancer vs healthy comparison, 58 (AAL), 88 (SNA) and 74 (STL) peptides were found, with an overlap of 37 peptides that were mapped to 18 proteins (FIG. 3).

Each glycoprotein candidate was measured by three or more non-glycosylated peptides, but not all peptides showed significance or consistent direction of change. Screening for consistent difference (up/down) across all measured peptides identified five proteins elevated in ovarian cancer (A1AT, AACT, CO9, HPT and ITIH3), and four down-regulated proteins (A2MG, ALS, IBP3 and PON1) in ovarian cancer (Table 6).

Glycoproteins with all targeted peptides increased or decreased in HGSOC clinical cohort with adjusted p-value <0.05 and log 2FC >0.5 for each lectin.

TABLE 6
BIOMARKER GLYCOPROTEINS WITH ALL CONSISTENT PEPTIDES

| LECTIN | Overexpressed | Underexpressed | TOTAL NUMBER |
|---|---|---|---|
| AAL | A1AT, AACT, CO9, HPT | A2MG | 5 |
| SNA | A1AT, CO9, ITIH3 | A2MG, ALS, IBP3 | 6 |
| STL | CO9, ITIH3 | A2MG, ALS, IBP3, PON1 | 6 |
| TOTAL | 5 | 4 | |
| NUMBER | | | |

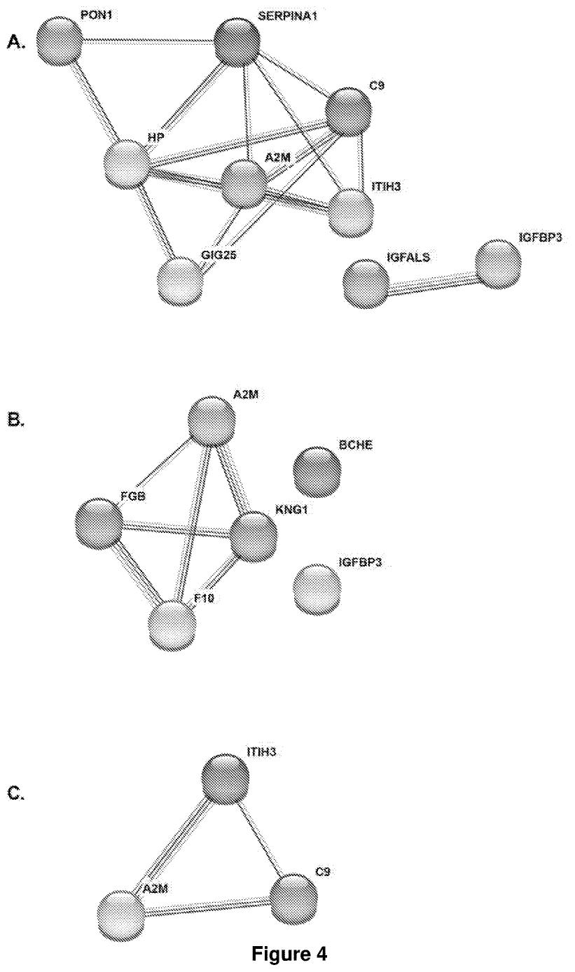

STRING v 11.5 was used to investigate the interactions between the nine validated biomarker proteins. The developed protein-protein interaction (PPI) network had 16 edges (expected 0), and significantly more interactions than expected (PPI enrichment p-value of <1×10⁻¹⁶) (FIG. 4A). Functional enrichment analyses revealed significant enrichments in Gene Ontology Cellular Component terms Blood microparticle (5 out of 115 genes, FDR 1.67×10⁻⁶), extracellular exosome (8 out of 2099 genes, FDR 8.21 1.67×10⁻⁵), insulin-like growth factor ternary complex (2 out of 4 genes, FDR 0.0048), amongst others, as well as KEGG pathway, Complement and coagulation cascades (3 out of 82 genes, FDR 0.0022).

Multi-marker signatures for detection of ovarian cancer were developed for each lectin and a combination of all three lectins. The receiver operating curve (AUC), specificity and sensitivity of the developed multi-peptide models are detailed in Table 6. All four models performed similarly with the AAL signature having the highest AUC (87.5%), sensitivity (70.4%) and specificity (90.7%). To further inspect the stable peptides for each of the models, we filtered peptides chosen in at least 50% of the cross-validation runs (Table 7). This analysis revealed several interesting observations. The IBP3 peptide ALAQCAPPPAVCAELVR was always selected for each lectin signature, indicating strong predictive value for HGSOC. Two peptides were highly stable for SNA, STL and the combined signatures, namely, A2MG_NEDSLVFVQTDK and CHLE_NIAAFGGNPK. For the combined signature, both SNA and STL binding IBP3_ALAQCAPPPAVCAELVR were selected with high stability (100% and 91.6%, respectively). Strikingly, the stable peptides in the combined signature comprised 2 SNA and 5 STL peptides, with no AAL peptides.

TABLE 7
SIGNATURE PEPTIDES FROM LECTIN-PULLDOWNS

| | AAL | SNA | STL | Combined |
|---|---|---|---|---|
| Lectin (sample size) | (n = 70) | (n = 95) | (n = 95) | (n = 95) |
| Model performance (range) | AUC = | AUC = | AUC = | AUC = |
| | 87.6% | 84.6% | 85.0% | 84.8% |
| | (78.4%, | (76.1%, | (76.6%, | (76.4%, |
| | 96.8%) | 93.0%) | 93.4%) | 93.3%) |
| | Sens = | Sens = | Sens = | Sens = |
| | 70.4% | 64.1% | 64.1% | 64.1% |
| | (51.5%, | (48.4%, | (48.4%, | (48.4%, |
| | 84.1%) | 77.3%) | 77.3%) | 77.3%) |
| | Spec = | Spec = | Spec = | Spec = |
| | 90.7% | 87.5% | 85.7% | 89.3% |
| | (78.4%, | (76.4%, | (74.3%, | (78.5%, |
| | 96.3%) | 93.8%) | 92.6%) | 95.0%) |
| | Times | Times | Times | Times |
| UniProt Name_Peptide | selected | selected | selected | selected |
| IBP3_ALAQCAPPPAVCAELVR | 70 | 95 | 95 | SNA 95 |
| | (100%) | (100%) | (100%) | (100%) |
| | | | | STL 87 |
| | | | | (91.6%) |
| A1AT_QINDYVEK | 70 | | | |
| | (100%) | | | |
| HPT_VTSIQDWVQK | 69 | | 83 | |
| | (98.5%) | | (87.4%) | |
| IBP3_FLNVLSPR | 69 | | | |
| | (98.5%) | | | |
| GELS_QTQVSVLPEGGETPLFK | 68 | | | |
| | (97.1%) | | | |
| FETUA_HTLNQIDEVK | 64 | | | |
| | (91.4%) | | | |
| FIBB_YQISVNK | 46 | | | |
| | (65.7%) | | | |
| FA10_ETYDFDIAVLR | | 94 | | |
| | | (98.9%) | | |
| A2MG_NEDSLVFVQTDK | | 93 | 95 | STL 95 |
| | | (97.9%) | (100%) | (100%) |
| CHLE_NIAAFGGNPK | | 93 | 67 | SNA 92 |
| | | (97.9%) | (70.5%) | (96.8%) |
| KNG1_ENFLFLTPDCK | | 93 | | |
| | | (97.9%) | | |
| ITIH2_VQSTITSR | | 90 | | |
| | | (94.7%) | | |
| ITIH3_EHLVQATPENLQEAR | | 86 | 91 | |
| | | (90.5%) | (95.8%) | |
| C1QC_QTHQPPAPNSLIR | | 85 | | |
| | | (89.5%) | | |
| HPT_DYAEVGR | | 84 | | |
| | | (88.4%) | | |
| THRB_SGIECQLWR | | 79 | | |
| | | (83.2%) | | |
| KNG1_EGDCPVQSGK | | | 95 | STL 95 |
| | | | (100%) | (100%) |
| FIBB_QDGSVDFGR | | | 78 | |
| | | | (82.1%) | |
| FA10_IVGGQECK | | | | STL 66 |
| | | | | (69.5%) |
| FIBB_SILENLR | | | | STL 60 |
| | | | | (63.2%) |

AUC, area under the receiver operating curve; Sens, sensitivity; Spec, specificity.

PPI analysis again revealed significant interactions with enrichment value of 1.29×10⁻⁷ (FIG. 4B), and enrichment of the GO Cellular Component term blood microparticle (4 out of 115 genes, FDR 3.28×10⁻⁵), as well as the KEGG pathway component and coagulation cascades (4 out of 82 genes, FDR 1.74×10⁻⁶). Additionally, the GO biological process blood coagulation, fibrin clot formation was also highly enriched (4 out of 26 genes, FDR 8.62×10⁻⁷).

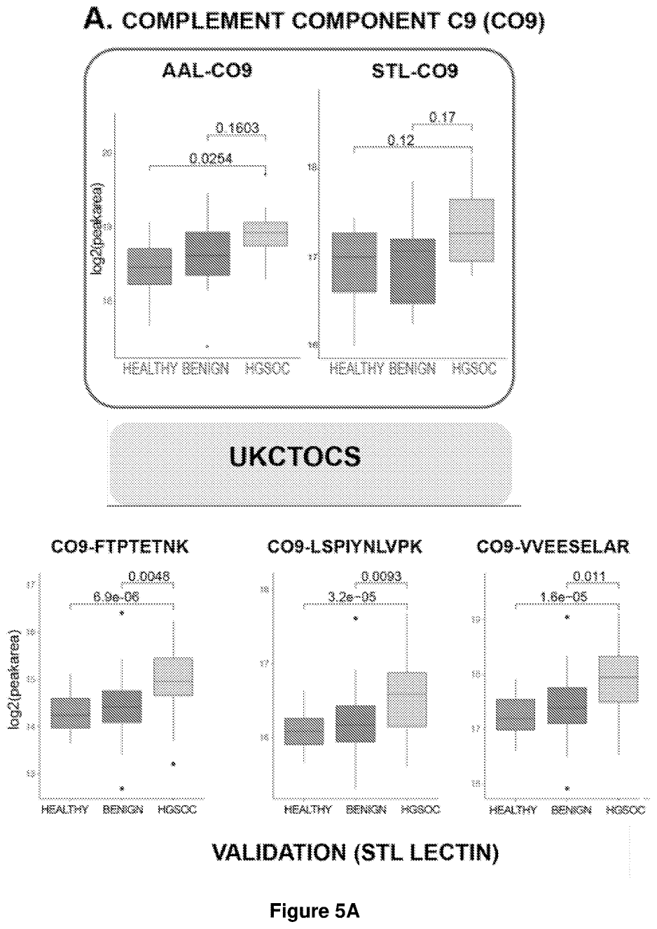

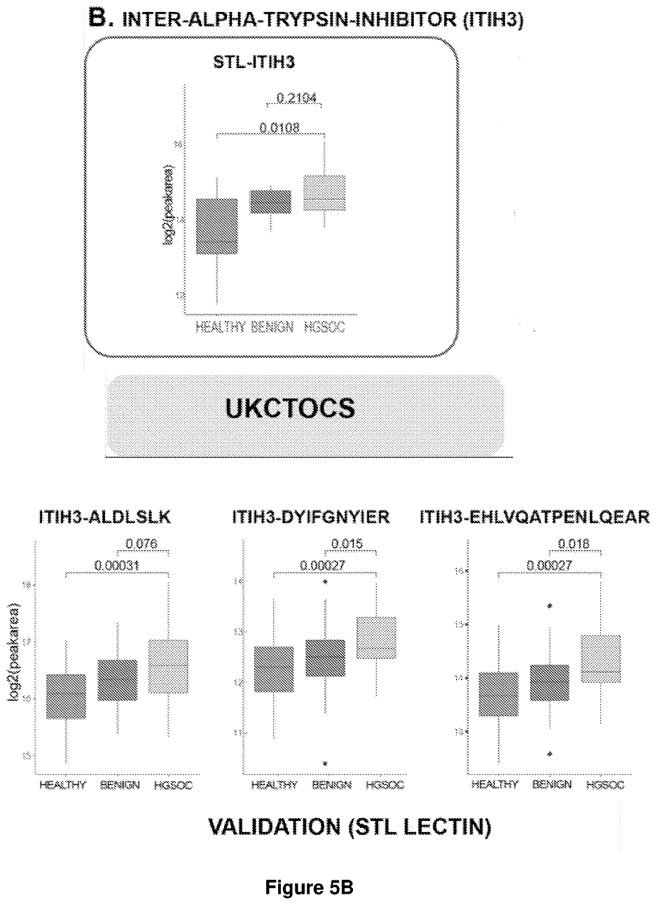

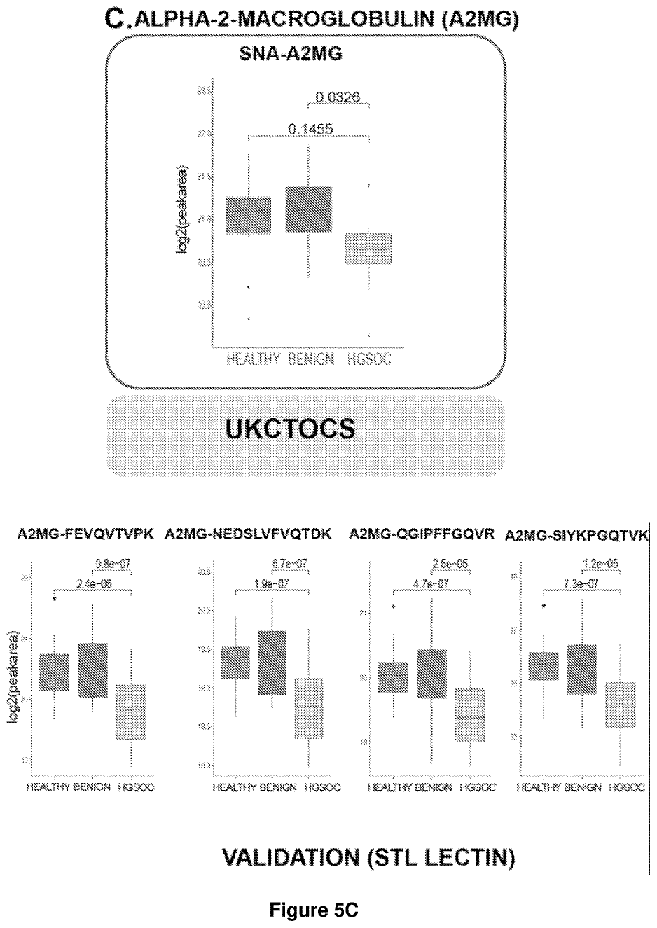

For use in ovarian cancer screening application, the biomarkers should be detectable in asymptomatic subjects. The serum samples from the longitudinal UKCTOCS cohort were collected prior to ovarian cancer diagnosis. On re-examination of the discovery data from UKCTOCS cohort, it was found that three of the nine validated univariate biomarkers were significantly altered at the preclinical time point, namely CO9, ITIH3 and A2MG. In the validation phase, CO9 and A2MG were significant for all three tested lectins, while ITIH3 was significant for STL and SNA (Table 5). FIG. 5 illustrates the comparative data from discovery UKCTOCS (protein level) and validation (peptide level, STL-pulldown) phases. AAL-CO9 (FIG. 5A) and STL-ITIH3 (FIG. 5B) were significantly higher in HGSOC group compared to benign and healthy groups in the UKCTOCS set, while SNA-A2MG was lower in HGSOC group (FIG. 5C). STL-CO9 was also elevated in HGSOC versus other groups in the UKCTOCS set, albeit not statistically significant (FIG. 5A), while STL-A2MG was not detected in the UKCTOCS or UKOPS datasets likely due to the lower sensitivity of DDA-MS compared to MRM-MS. The three early detection biomarkers CO9, ITIH3 and A2MG interacted in a tight network (enrichment p-value 3.91×10⁻⁶) (FIG. 4C), which was functionally enriched in the KEGG pathway Complement and coagulation cascades (FDR 0.00183) and in the UniProt annotated keyword of Serine protease inhibitors (FDR 0.0351).

Using the glycosylation-focussed biomarker pipeline, the inventors discovered and validated serum biomarkers to facilitate ovarian cancer detection through blood testing. Interestingly, several glycoproteins show promise as predictive biomarkers, as they are already altered in serum samples collected 4-18 months prior to ovarian cancer diagnosis.

In addition to the intended utility as blood biomarkers for ovarian cancer detection, the study also revealed intriguing biology in early HGSOC. Protein-protein interaction and functional analysis of the validated glycoprotein biomarkers for HGSOC, as well as the three HGSOC predictive markers concurs with the known hypercoagulability of ovarian cancer (Weeks, Herbach, McDonald, Charlton, & Schweizer, 2020), and implicate blood microparticles. Interestingly, while venous thromboembolisms (VTEs) is mostly highly associated with advanced ovarian cancer and clear cell histology (Weeks et al., 2020), here the inventors identified an enrichment in complement and coagulation cascade in differential diagnosis and predictive HGSOC serum glycoprotein biomarkers. Both sets of biomarkers showed an enrichment in blood microparticles, which is defined by Gene Ontology as a type of extracellular vesicle (EV) devoid of nucleic acids, released from several cell types including platelets and endothelial cells. While the glycoprotein workflow did not isolate EV from sera, the inventors conducted denaturation of the serum sample in the first step, which will have solubilised all EV proteins allowing them to be analysed along with other serum glycoproteins. The findings of this study corroborates with two previous independent cohort studies reporting elevated serum/plasma EV procoagulant activity in ovarian cancer patients (Claussen et al., 2016; Zhang, 2019), and further suggest a potential role of EV complement/coagulation proteins in tumour initiation with the finding of early elevation prior to cancer diagnosis.

One of the top glycoprotein biomarkers discovered in this study, complement component C9, has also been found to be elevated in serum of gastric (Chong et al., 2010), lung (Narayanasamy et al., 2011), colorectal (Chantaraamporn et al., 2020; Murakoshi et al., 2011) and oesophageal cancers (Shah et al., 2015; Shah et al., 2018) through proteomics or glycoproteomic approaches. C9 is the terminal component of the complement cascade, and is the pore-forming protein of the membrane attack complex. The bulk of serum C9 is synthesised and secreted from hepatocytes, and diverse serum C9 glycoforms have been mapped (Franc, Yang, & Heck, 2017). In a previous oesophageal adenocarcinoma biomarker study, C9 glycoforms binding to each of the 6 short-listed lectins (AAL, EPHA, JAC, NPL, PSA, WGA) was found to be significantly elevated in oesophageal adenocarcinoma compared to the precursor benign condition, Barrett's oesophagus but with some variability in healthy groups (Shah et al., 2015). Recently, the inventors reported the release of C9+EV by oesophageal adenocarcinoma cells as a potential mechanism of the elevated serum C9 glycoform in oesophageal cancer (Kolka et al., 2022). However, the specific glycosylation differences in serum and on EVs, as well as the molecular mechanisms underpinning the altered C9 glycosylation in cancer remains to be determined. The current study has shortlisted three lectins (AAL, SNA, STL) for the validation phase, and all three C9 glycoforms measured were significantly elevated in HGSOC. Interestingly, in addition to oesophageal adenocarcinoma (Shah et al., 2015; Shah et al., 2018), AAL-binding (indicative of fucosylated) C9 was reported to be elevated in lung (squamous and adenocarcinoma) and stomach cancers with intermediate levels found in hepatocellular carcinoma and low levels in breast cancer (Narayanasamy et al., 2011).

In addition to AAL-C9 and STL-C9, results revealed that AAL-ITIH3 and STL-ITIH3 are also elevated prior to HGSOC diagnosis, while serum SNA-A2MG wase already lowered prior to HGSOC diagnosis. Of these, only A2MG has previously been reported as diagnostic in ovarian cancer. The inter-alpha-trypsin inhibitor family has been implicated in inflammation and carcinogenesis (Hamm et al., 2008). In addition to its protease inhibitor activity, ITIH3 is thought to stabilise the extracellular matrix through binding to hyaluronic acid (Huang, Yoneda, & Kimata, 1993). Interestingly, previous reports suggest blood ITIH3 to be elevated for a similar range of cancers as C9, namely, lung (Heo, Lee, Ryoo, Park, & Cho, 2007), gastric (Chong, Lee, Zhou, et al., 2010), pancreatic (Liu et al., 2017) and colorectal (Kopylov et al., 2020) cancers. Alpha-2 macroglobulin is a broad-spectrum protease inhibitor with structural and functional similarity to complement component 3 (C3), and directly interacts with several components of the complement system (Vandooren, 2021). Decreased serum A2MG protein was previous reported in ovarian cancer compared to healthy controls (Miyamoto et al., 2018). The same study also measured four A2MG glycopeptides using targeted mass spectrometry with mixed results (Miyamoto et al., 2018). Alternation in A2MG glycosylation has been reported in colorectal cancer using lectin microarray or lectin-assisted glycoproteomics (Chantaraamporn, 2020; Sunderic, 2016).

The LeMBA-MRM mass spectrometry assay reported in this study can be deployed as a lab-developed test to measure a biomarker panel following lectin pulldown. Alternatively, lectin-immunoassays have also been reported for AAL-C9 (Narayanasamy, 2011) and JAC-C9 (Webster, 2021) using microplate and microfluidic immunoassay formats, respectively. In inventor's previous study (Webster, 2021), they compared the diagnostic value of total serum C9 and JAC-C9 in detecting oesophageal adenocarcinoma from benign (Barrett's oesophagus) and healthy in the same cohort, and found JAC-C9 to have a higher diagnostic value for cancer. Therefore, it is envisaged that lectin-assisted assays will provide the throughput and specificity required for ovarian cancer detection. The three final lectins (AAL, SNA and STL) taken to the validation phase suggest alterations in a-fucose, sialic acid and N-acetylglucosamine during HGSOC development. While the STL panel provided the highest diagnostic power in the validation cohort, ideally, additional cohorts are evaluated against all three lectins to ensure selection of the most robust panel, accounting for population differences.

In conclusion, the discovery and validation of serum glycoprotein markers for HGSOC using lectin-assisted workflow is reported to be directly translatable to blood tests. The validated markers show high specificity when bench marked against CA125. Furthermore, several markers were elevated months prior to cancer diagnosis, and should be further evaluated as predictive markers. Functional enrichment of the validated markers highlights blood microparticle (EV)-mediated complement and coagulation activity, which appears to be elevated prior to HGSOC diagnosis. Further investigation on the contribution of EV-mediated complement and coagulation in ovarian cancer development may provide mechanisms for prevention.

### Materials & Methods

### Study Design

The 2-phase biomarker study design using LeMBA-MS is illustrated in FIG. 1. Biomarker discovery phase used 7 lectins: Aleuria aurantia (AAL), Concanavalin-A (Con-A), Erythrina cristagalli (ECA), Phaseolus vulgaris Leucoagglutinin (L-PHA), Sambucus nigra (SNA), Solanum tuberosum (STL) and Wisteria floribunda (WFA), which preferentially target glycoproteins with fucose, mannose, galactose, branched chain glycans, sialic acid, N-acetylglucosamine, and N-acetylgalactosamine, respectively.

Two cohorts were used in the discovery phase, each comprising of 30 age-matched serum samples from ovarian cancer (n=10), benign (n=10) and healthy control (n=10) groups, total 60 samples. Ovarian cancer serum samples from United Kingdom Collaborative Trial of Ovarian Cancer Screening (UKCTOCS) cohort were collected 4-18 months prior to cancer diagnosis, while the United Kingdom Ovarian Population Study (UKOPS) samples were collected within four months prior to diagnosis. A shortlist of candidate proteins and lectins was generated from the discovery data, for validation in a second cohort of 95 serum samples from the Australian Ovarian Cancer Study (AOCS). The cohort characteristics for UKOPS and UKCTOCS are shown in Table 8 and AOCS cohort characteristics shown in Table 9.

TABLE 8
CLINICAL INFORMATION FOR THE DISCOVERY PHASE COHORT

| | UKOPS | | | UKCTOCS | | |
|---|---|---|---|---|---|---|
| | | | Ovarian | | | Ovarian |
| | Healthy | Benign | cancer | Healthy | Benign | cancer |
| Samples | 10 | 10 | 10 | 10 | 10 | 10 |
| Age | 59.5 ± 5.9 | 59.4 ± 6.1 | 59.9 ± 6.3 | 65.2 ± 6.1 | 65.7 ± 5.9 | 65.0 ± 6.0 |
| (median) | | | | | | |
| BMI | 25.4 ± 4.0 | 24.9 ± 3.0 | 24.8 ± 3.5 | 25.6 ± 3.0 | 24.85 ± 2.9 | 28.6 ± 5.0 |
| (median) | | | | | | |
| Smoking | | | | | | |
| history | | | | | | |
| Current | 1 (10%) | 0 (0%) | 4 (40%) | 1 (10%) | 2 (20%) | |
| Former | 1 (10%) | 3 (30%) | 1 (10%) | | | |
| Never | 6 (60%) | 4 (40%) | 3 (30%) | 7 (70%) | 4 (40%) | 1 (10%) |
| Don't know | 2 (20%) | 3 (30%) | 2 (20%) | 2 (20%) | 2 (20%) | 9 (90%) |
| Alcohol | | | | | | |
| status | | | | | | |
| Current | 6 (60%) | 6 (60%) | 7 (70%) | 5 (50%) | 4 (40%) | |
| Not current | 2 (20%) | 0 (0%) | 1 (10%) | 1 (10%) | 2 (20%) | 1 (10%) |
| Don't know | 2 (20%) | 4 (40%) | 2 (20%) | 4 (40%) | 2 (20%) | 9 (90%) |

TABLE 9
AUSTRALIAN OVARIAN CANCER STUDY
(AOCS) COHORT INFORMATION

| Clinical Parameters | Healthy | Benign | Ovarian cancer |
|---|---|---|---|
| Samples | 28 | 28 | 39 |
| Age (median ± SD) | 61.0 ± 10.8 | 60.0 ± 10.4 | 59 ± 10.4 |
| Age at last menopause (median ± SD) | — | 45 ± 7.5 | 49.5 ± 6.3 |
| BMI (median ± SD) | — | 26.2 ± 7.7 | 28.4 ± 6.9 |
| Hysterectomy | | | |
| Yes | — | 9 (32.14%) | 8 (20.51%) |
| No | | 19 (67.85%) | 31 (79.48%) |
| Age at hysterectomy (Median ± SD) | — | 34 (4.470) | 44 (10.582) |
| Smoking history | | | |
| Current | — | — | 6 (15.38%) |
| Former | | | 11 (28.20%) |
| Never | | | 21 (53.84%) |
| Don't know | | | 1 (2.56%) |

### Serum Denaturation

Patient serum samples (n=60) were centrifuged at 16,000 g at 4° C. for 15 min to remove cellular debris and the supernatant protein concentration was determined using BCA protein assay. To minimise batch effects, bulk serum denaturation was performed for the entire project. An aliquot of each serum sample containing 800 μg of protein was diluted to 10 μg/uL in denaturation buffer (20 mM Tris pH 7.4, 1% v/v sodium dodecyl sulfate (SDS) and 5% v/v Triton X-100). The internal standard protein chicken ovalbumin was added to each serum sample at 10 μmol. Protein disulfide bonds were reduced by adding 20 mM dithiothreitol to the samples and incubating at 37° C. for 30 min. Following this, 100 mM iodoacetamide was added to the sample and incubated at room temperature for 30 min in the dark to alkylate free thiol groups. The denatured serum samples were diluted 20 times in LeMBA binding buffer (20 mM Tris, pH 7.4+300 mM NaCl+1 mM CaCl₂)+1 mM MnCl₂+1% Triton+1 unit protease inhibitor cocktail, pH 7.4) to yield a final protein concentration of 0.5 μg/μL. Aliquots of 100 L were transferred to microplates in a randomized layout in preparation for LeMBA. Prepared plates were sealed and frozen at −80° C. until use.

### Lectin Magnetic Bead Pulldown and On-Bead Trypsin Digest

LeMBA-MS with the selected 7 lectins was performed as previously described (Choi et al., 2011; Shah et al., 2015). Firstly, individual lectins were conjugated to MyOne™ tosyl-activated DYNABEADS™ by incubating 50 μg of lectin with 100 μL of Dynabeads™ at 37° C. for 24 h. The resulting lectin-bead conjugate was treated with 2% w/v glycine solution to reduce nonspecific binding, and further incubated at 37° C. for 16 h. The blocked beads were washed and diluted in lectin storage buffer (20 mM Tris, pH 7.4+150 mM NaCl+1 mM CaCl₂)+1 mM MnCl₂+0.5% Triton-X 100+1 unit protease inhibitor cocktail).

Pulldown using the prepared lectin magnetic beads was performed on the AssayMAP Bravo liquid handler workstation using one lectin per microplate. Briefly, 50 μL conjugated beads and 100 μL denatured serum was added to each well of a 96-well microtiter plate, and glycoprotein capture was performed at 4° C. for 1 h under gentle shaking. Post incubation, the conjugated beads with the captured glycoproteins were washed seven times in 50 mM ammonium bicarbonate buffer with two microplate changes to minimise trace detergent, reducing and alkylating agent concentrations. The captured glycoproteins were digested with trypsin at 37° C. for 18 h after adding 50 mM ammonium bicarbonate buffer and 1 μg of sequencing grade porcine trypsin to each well. The plate was sealed for enzyme digestion. The trypsin was inactivated with 1% v/v formic acid (FA) and the digested peptides were collected, dried down in a vacuum concentrator, sealed and stored at −80° C. until use.

### Data Dependent Acquisition and Spectral Library Search

Shotgun proteomics using data-dependent acquisition was performed on a SCIEX 5600 Triple TOF 5600+mass spectrometer coupled to a Shimadzu LC-20AD Prominence nano liquid chromatography system. All solvents and reagents used were of mass spectrometry grade. The mass spectrometer was controlled using Analyst 1.7 software (AB SCIEX). Digested peptides were resuspended in 0.1% v/v FA and injected onto a Protecol C18 analytical column (200 Å, 3 μm, 150 mm×150 μm) connected to a Protecol guard column (Polar 120 Å, 3 μm, 10 mm×300 μm) and the sample injection order was randomised in the worklist. Column compartment was maintained at 45° C. The peptides were eluted using mobile phase A (0.1% v/v FA) over the specified gradient of mobile phase B (95% acetonitrile, 5% v/v water, 0.1% v/v FA) for 60 min at a flow rate of 1.2 μL/min (5% B at 3 min; 30% B at 37 min; 50% B at 45 min; 100% B at 47 min; 100% B at 51 min; 5% B at 53 min until end of run). The nanospray ion source was set as follows: ion source gas 1=35 psi, curtain gas=30 psi, ion spray floating voltage=2400 V and interface heater temperature=180° C. The ion optics parameters were set as declustering potential=100 V and collision energy (survey scan)=10 V. Data acquisition was performed using the information dependent acquisition (IDA) and top 30 precursors from each survey scan were selected for fragmentation. The MS1 spectra was acquired in positive polarity within the mass range=m/z 350-1250 Da, with the accumulation time of 250 ms. The precursor selection mass window in the quadrupole was set to unit resolution (m/z 0.7 window). The MS/MS spectra were acquired using collision induced dissociation (CID) within the mass range=m/z 100-1500 Da with the following parameters: charge states+2 to +5, accumulation time=100 ms, fragmentation threshold=150 cps, dynamic exclusion=15 s and collision energy voltage was set as rolling collision energy with a collision energy spread of 3.

The acquired raw ion spectra for each lectin batch were searched for protein IDs against the reviewed UniProt human proteome database (20,365 proteins, accession date Jan. 1, 2020) using MaxQuant software, v. 1.6.6.0 (Cox & Mann, 2008). The MaxQuant contaminant database (247 entries) was also searched to identify contaminants such as keratin. MaxQuant parameters were set as follows: Digestion=trypsin, with 2 missed cleavages; fixed modification was set to cysteine carbamidomethylation; variable modifications were set as methionine oxidation and N-terminal acetylation; Label free quantification (LFQ) was enabled with minimum ratio count set to 2; unique and razor peptides were used for protein identification; match between runs was set as TRUE; and false discovery rate (FDR) for protein and peptide identification was set at 0.01. AB Sciex Q-TOF was set as instrument type using default settings. First search peptide tolerance was set to 0.07 Da and 0.06 Da for the main search. MS/MS tolerance was set at 40 ppm. The search results were imported into R v software v1.4.1103 for further data processing and statistical analyses.

### Data Processing and Statistical Analysis

The generated protein list for each lectin batch was filtered to remove contaminants, reverse identified protein IDs, proteins with <2 peptide IDs and score <5. Proteins which were missing in <25% of all samples were considered missing at random and imputed using localized least square regression (Ilsimpute) (Kim, Golub, & Park, 2005). Proteins missing in >25% were imputed with the minimum detected value (values drawn randomly from a normal distribution centred at sample minimum and with SD estimated from non-missing proteins). Log₂ transformed data was analysed using the R limma package to identify differentially abundant proteins. All graphical output has been generated using R and figures prepared using Illustrator and Biorender.

### Multiple Reaction Monitoring Assay Development

Multiple reaction monitoring (MRM) assay development was done using Skyline v 21.1.0.278 (http://skyline maccosslab.org/). The discovery phase raw spectral files (n=420) were uploaded to Skyline to generate a unified spectral library for MRM method development. The human proteome was selected as a background proteome and trypsin was selected to perform in silico digest of library peptides. The biomarker candidate list generated from the discovery phase for both TOCS and OPS cohorts was imported into Skyline and the initial transition selection was done based on spectral library matching. Immunoglobulins were manually removed from candidate list.

For each candidate biomarker protein, a minimum of ten peptides were selected, each consisting of at least six transitions (b and y ions). Additionally, the “Unique Peptides” parameter in Skyline was applied to check for peptide uniqueness to a single protein. Digested peptide samples from all the lectin batches and across both discovery cohorts were pooled into a single sample that was used for MRM method development. To monitor retention time across all runs, 3 stable isotope standard (SIS) peptides (VTSIQDWVQK, NLAVSQVVHK, LSPIYNLVPVK) were added. The final dynamic MRM method consisted of 60 proteins (59 candidate biomarker proteins+1 chicken ovalbumin protein), 176 peptides (170 candidate peptides+3 SIS peptides+3 chicken ovalbumin peptides) and 860 transitions with a delta retention time of 1 min.

### LeMBA-MRM-MS

Candidate biomarker validation was performed on patient serum samples (n=95; healthy=28, benign=28, HGOSC=39) from an independent cohort from AOCS. Based on the discovery phase results, AAL, SNA and STL lectins were selected for the validation phase and the serum samples were subjected to the LeMBA workflow as described above. Prior to mass spectrometry injection, three SIS peptides (VTSIQDWVQK, NLAVSQVVHK, LSPIYNLVPVK) were spiked-in to the samples for monitoring retention time stability across runs. Multiple reaction monitoring-mass spectrometry (MRM-MS) was performed on an Agilent 6490 triple quadrupole mass spectrometer coupled to an Agilent 1290 Infinity UHPLC system, equipped with an Agilent jet stream+ESI source. The mass spectrometer was controlled by MassHunter software (Agilent). 10 μL (20 μg) of digested peptides was injected onto a reverse phase AdvanceBio Peptide Mapping analytical column (150×2.1 mm i.d., 2.7 μm, part number 653750-902) connected to a 5 mm long guard column and the sample injection order was randomised in the worklist. The column compartment was maintained at 50° C. The peptides were eluted using mobile phase A (0.1% v/v FA) over the specified gradient of mobile phase B (100% acetonitrile, 0.1% v/v FA) for 35 min at a flow rate of 0.4 mL/min (3% B at 0 min; 30% B at 20 min; 40% B at 24 min; 95% B at 24.5 min; 95% B at 28.5 min and 3% B at 29 min until end of run). The mass spectrometer operated in positive ion mode and the source parameters were set as 150 V high pressure RF, 60 V low pressure RF, 4000 V capillary voltage, 300 V nozzle voltage, 30 psi nebulizer gas flow, 15 L/min drying gas flow at a temperature of 150° C., 11 L/min sheath gas flow at a temperature of 250° C. and 200 V delta EMV. The quadrupole was set at unit resolution (0.7 Da full width at half maximum in the first quadrupole (Q1) and the third quadrupole (Q3)), fragmentor at 380 V and cell accelerator voltage at 4 V.

### Data Analysis for Lectin MRM-MS

The data analysis for each lectin MRM-MS was performed independently with no comparisons performed across the three lectins. MRM-MS raw data for the three lectins were exported to Skyline v 21.1.0.278 (downloaded January 2022, http://skyline.maccosslab.org/) to manually check for correct peak integration and the peak area of each measured transition was exported and further analysed on R v1.4.1103. The peak area distribution for the monitored internal (ovalbumin chicken peptides) and external (SIS peptides) standards were checked to detect any outliers in the dataset. For all three dataset, peak area distribution for the standards was robust and within a range of 30-40% CV. The peak area distribution of all the samples was also within range exhibiting a normal data distribution, thus not requiring application of data normalisation methods to correct for data distribution. For each measured peptide in each sample, the transition peak areas were summed and then Log₂ transformed. Student's T-test was performed between the clinical cohorts and false discovery rate using Benjamini-Hochberg method was applied to identify significantly differing proteins at p adjusted-value <0.05. All graphical output has been generated using R and figures prepared using Illustrator and Biorender.

### Multimarker Panel Development

Generalized regression with binomial distribution and lasso estimation was used to develop multimarker panels using JMP Pro version 16.2.0, with leave-one-out validation. Performance of the multimarker panels were assessed using area under the receiver operating curve, specificity, sensitivity and the number of times each parameter (peptide) was chosen in cross-validation models.

The disclosure of every patent, patent application, and publication cited herein is hereby incorporated herein by reference in its entirety.

The citation of any reference herein should not be construed as an admission that such reference is available as “prior art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Alam, S., Anugraham, M., Huang, Y. L., Kohler, R. S., Hettich, T., Winkelbach, K., Jacob, F. (2017). Altered (neo-) lacto series glycolipid biosynthesis impairs alpha2-6 sialylation on N-glycoproteins in ovarian cancer cells. Sci Rep, 7, 45367.
Bayoumy, S., Hyytia, H., Leivo, J., Talha, S. M., Huhtinen, K., Poutanen, M., Pettersson, K. (2020). Glycovariant-based lateral flow immunoassay to detect ovarian cancer-associated serum CA125. Commun Biol, 3(1), 460.
Biskup, K., Braicu, E. I., Sehouli, J., Fotopoulou, C., Tauber, R., Berger, M., & Blanchard, V. (2013). Serum glycome profiling: a biomarker for diagnosis of ovarian cancer. J Proteome Res, 12(9), 4056-4063.
Biskup, K., Braicu, E. I., Sehouli, J., Tauber, R., & Blanchard, V. (2014). The serum glycome to discriminate between early-stage epithelial ovarian cancer and benign ovarian diseases. Dis Markers, 2014, 238197.
Biskup, K., Braicu, E. I., Sehouli, J., Tauber, R., & Blanchard, V. (2017). The ascites N-glycome of epithelial ovarian cancer patients. J Proteomics, 157, 33-39.
Chantaraamporn, J., Champattanachai, V., Khongmanee, A., Verathamjamras, C., Prasongsook, N., Mingkwan, K., Svasti, J. (2020). Glycoproteomic Analysis Reveals Aberrant Expression of Complement C9 and Fibronectin in the Plasma of Patients with Colorectal Cancer. Proteomes, 8(3).
Choi, E., Loo, D., Dennis, J. W., O'Leary, C. A., & Hill, M. M. (2011). High-throughput lectin magnetic bead array-coupled tandem mass spectrometry for glycoprotein biomarker discovery. Electrophoresis, 32(24), 3564-3575.
Chong, P. K., Lee, H., Loh, M. C., Choong, L. Y., Lin, Q., So, J. B., Lim, Y. P. (2010). Upregulation of plasma C9 protein in gastric cancer patients. Proteomics, 10(18), 3210-3221.
Chong, P. K., Lee, H., Zhou, J., Liu, S. C., Loh, M. C., Wang, T. T., Lim, Y. P. (2010). ITIH3 is a potential biomarker for early detection of gastric cancer. J Proteome Res, 9(7), 3671-3679.
Claussen, C., Rausch, A. V., Lezius, S., Amirkhosravi, A., Davila, M., Francis, J. L., Langer, F. (2016). Microvesicle-associated tissue factor procoagulant activity for the preoperative diagnosis of ovarian cancer. Thromb Res, 141, 39-48.
Cox, J., & Mann, M. (2008). MaxQuant enables high peptide identification rates, individualized p.p.b.-range mass accuracies and proteome-wide protein quantification. Nat Biotechnol, 26(12), 1367-1372.
Dedova, T., Braicu, E. I., Sehouli, J., & Blanchard, V. (2019). Sialic Acid Linkage Analysis Refines the Diagnosis of Ovarian Cancer. Front Oncol, 9, 261.
Franc, V., Yang, Y., & Heck, A. J. (2017). Proteoform Profile Mapping of the Human Serum Complement Component C9 Revealing Unexpected New Features of N—, O—, and C-Glycosylation. Anal Chem, 89(6), 3483-3491.
Gidwani, K., Huhtinen, K., Kekki, H., van Vliet, S., Hynninen, J., Koivuviita, N., Pettersson, K. (2016). A Nanoparticle-Lectin Immunoassay Improves Discrimination of Serum CA125 from Malignant and Benign Sources. Clin Chem, 62(10), 1390-1400.
Hamm, A., Veeck, J., Bektas, N., Wild, P. J., Hartmann, A., Heindrichs, U., Dahl, E. (2008). Frequent expression loss of Inter-alpha-trypsin inhibitor heavy chain (ITIH) genes in multiple human solid tumours: a systematic expression analysis. BMC Cancer, 8, 25.
Hayashi, M., Matsuo, K., Tanabe, K., Ikeda, M., Miyazawa, M., Yasaka, M., Mikami, M. (2019). Comprehensive Serum Glycopeptide Spectra Analysis (CSGSA): A Potential New Tool for Early Detection of Ovarian Cancer. Cancers (Basel), 11(5).
Henderson, J. T., Webber, E. M., & Sawaya, G. F. (2018). Screening for Ovarian Cancer: Updated Evidence Report and Systematic Review for the US Preventive Services Task Force. JAMA, 319(6), 595-606.
Heo, S. H., Lee, S. J., Ryoo, H. M., Park, J. Y., & Cho, J. Y. (2007). Identification of putative serum glycoprotein biomarkers for human lung adenocarcinoma by multilectin affinity chromatography and LC-MS/MS. Proteomics, 7(23), 4292-4302.
Huang, L., Yoneda, M., & Kimata, K. (1993). A serum-derived hyaluronan-associated protein (SHAP) is the heavy chain of the inter alpha-trypsin inhibitor. J Biol Chem, 268(35), 26725-26730.
Huttenhain, R., Choi, M., Martin de la Fuente, L., Oehl, K., Chang, C. Y., Zimmermann, A. K., Aebersold, R. (2019). A Targeted Mass Spectrometry Strategy for Developing Proteomic Biomarkers: A Case Study of Epithelial Ovarian Cancer. Mol Cell Proteomics, 18(9), 1836-1850.
Kim, H., Golub, G. H., & Park, H. (2005). Missing value estimation for DNA microarray gene expression data: local least squares imputation. Bioinformatics, 21(2), 187-198.
Kohler, R. S., Anugraham, M., Lopez, M. N., Xiao, C., Schoetzau, A., Hettich, T., Heinzelmann-Schwarz, V. (2016). Epigenetic activation of MGAT3 and corresponding bisecting GlcNAc shortens the survival of cancer patients. Oncotarget, 7(32), 51674-51686.
Kolka, C. M., Webster, J., Lepletier, A., Winterford, C., Brown, I., Richards, R. S., Hill, M. M. (2022). C5b-9 Membrane Attack Complex Formation and Extracellular Vesicle Shedding in Barrett's Esophagus and Esophageal Adenocarcinoma. Front Immunol, 13, 842023.
Kopylov, A. T., Stepanov, A. A., Malsagova, K. A., Soni, D., Kushlinsky, N. E., Enikeev, D. V., Kaysheva, A. L. (2020). Revelation of Proteomic Indicators for Colorectal Cancer in Initial Stages of Development. Molecules, 25(3)
Kuzmanov, U., Musrap, N., Kosanam, H., Smith, C. R., Batruch, I., Dimitromanolakis, A., & Diamandis, E. P. (2013). Glycoproteomic identification of potential glycoprotein biomarkers in ovarian cancer proximal fluids. Clin Chem Lab Med, 51(7), 1467-1476.
Liu, X., Zheng, W., Wang, W., Shen, H., Liu, L., Lou, W., Yang, P. (2017). A new panel of pancreatic cancer biomarkers discovered using a mass spectrometry-based pipeline. Br J Cancer, 117(12), 1846-1854.
Lu, J., Isaji, T., Im, S., Fukuda, T., Kameyama, A., & Gu, J. (2016). Expression of N-Acetylglucosaminyltransferase III Suppresses alpha2,3-Sialylation, and Its Distinctive Functions in Cell Migration Are Attributed to alpha2,6-Sialylation Levels. J Biol Chem, 291(11), 5708-5720.
Menon, U., Gentry-Maharaj, A., Burnell, M., Singh, N., Ryan, A., Karpinskyj, C., Parmar, M. (2021). Ovarian cancer population screening and mortality after long-term follow-up in the UK Collaborative Trial of Ovarian Cancer Screening (UKCTOCS): a randomised controlled trial. Lancet, 397(10290), 2182-2193.
Mitra, I., Alley, W. R., Jr., Goetz, J. A., Vasseur, J. A., Novotny, M. V., & Jacobson, S. C. (2013). Comparative profiling of N-glycans isolated from serum samples of ovarian cancer patients and analyzed by microchip electrophoresis. J Proteome Res, 12(10), 4490-4496.
Miyamoto, S., Ruhaak, L. R., Stroble, C., Salemi, M. R., Phinney, B., Lebrilla, C. B., & Leiserowitz, G. S. (2016). Glycoproteomic Analysis of Malignant Ovarian Cancer Ascites Fluid Identifies Unusual Glycopeptides. J Proteome Res, 15(9), 3358-3376.
Miyamoto, S., Stroble, C. D., Taylor, S., Hong, Q., Lebrilla, C. B., Leiserowitz, G. S., Ruhaak, L. R. (2018). Multiple Rovarian cancertion Monitoring for the Quantitation of Serum Protein Glycosylation Profiles: Application to Ovarian Cancer. J Proteome Res, 17(1), 222-233.
Muinao, T., Deka Boruah, H. P., & Pal, M. (2019). Multi-biomarker panel signature as the key to diagnosis of ovarian cancer. Heliyon, 5(12), e02826.
Murakoshi, Y., Honda, K., Sasazuki, S., Ono, M., Negishi, A., Matsubara, J., Yamada, T. (2011). Plasma biomarker discovery and validation for colorectal cancer by quantitative shotgun mass spectrometry and protein microarray. Cancer Sci, 102(3), 630-638.
Narayanasamy, A., Ahn, J. M., Sung, H. J., Kong, D. H., Ha, K. S., Lee, S. Y., & Cho, J. Y. (2011). Fucosylated glycoproteomic approach to identify a complement component 9 associated with squamous cell lung cancer (SQLC). J Proteomics, 74(12), 2948-2958.
Nash, Z., & Menon, U. (2020). Ovarian cancer screening: Current status and future directions. Best Pract Res Clin Obstet Gynaecol, 65, 32-45.
Oungsakul, P., Choi, E., Shah, A. K., Mohamed, A., O'Leary, C., Duffy, D., Bielefeldt-Ohmann, H. (2021). Candidate Glycoprotein Biomarkers for Canine Visceral Hemangiosarcoma and Validation Using Semi-Quantitative Lectin/Immunohistochemical Assays. Vet Sci, 8(3).
Perez-Riverol, Y., Bai, J., Bandla, C., Garcia-Seisdedos, D., Hewapathirana, S., Kamatchinathan, S., Vizcaino, J. A. (2022). The PRIDE database resources in 2022: a hub for mass spectrometry-based proteomics evidences. Nucleic Acids Res, 50 (D1), D543-D552.
Pinho, S. S., & Reis, C. A. (2015). Glycosylation in cancer: mechanisms and clinical implications. Nat Rev Cancer, 15(9), 540-555.
Pinsky, P. F., Yu, K., Kramer, B. S., Black, A., Buys, S. S., Partridge, E., Prorok, P. C. (2016). Extended mortality results for ovarian cancer screening in the PLCO trial with median 15 years follow-up. Gynecol Oncol, 143(2), 270-275.
Schultz, M. J., Holdbrooks, A. T., Chakraborty, A., Grizzle, W. E., Landen, C. N., Buchsbaum, D. J., Bellis, S. L. (2016). The Tumour-Associated Glycosyltransferase ST6Gal-I Regulates Stem Cell Transcription Factors and Confers a Cancer Stem Cell Phenotype. Cancer Res, 76(13), 3978-3988.
Shah, A. K., Cao, K. A., Choi, E., Chen, D., Gautier, B., Nancarrow, D., Hill, M. M. (2015). Serum Glycoprotein Biomarker Discovery and Qualification Pipeline Reveals Novel Diagnostic Biomarker Candidates for Esophageal Adenocarcinoma. Mol Cell Proteomics, 14(11), 3023-3039.
Shah, A. K., Hartel, G., Brown, I., Winterford, C., Na, R., Cao, K. L., Hill, M. M. (2018). Evaluation of Serum Glycoprotein Biomarker Candidates for Detection of Esophageal Adenocarcinoma and Surveillance of Barrett's Esophagus. Mol Cell Proteomics, 17(12), 2324-2334.
Sheta, R., Bachvarova, M., Plante, M., Gregoire, J., Renaud, M. C., Sebastianelli, A., Bachvarov, D. (2017). Altered expression of different GalNActransferases is associated with disease progression and poor prognosis in women with high-grade serous ovarian cancer. Int J Oncol, 51(6), 1887-1897.
Sunderic, M., Sediva, A., Robajac, D., Miljus, G., Gemeiner, P., Nedic, O., & Katrlik, J. (2016). Lectin-based protein microarray analysis of differences in serum alpha-2-macroglobulin glycosylation between patients with colorectal cancer and persons without cancer. Biotechnol Appl Biochem, 63(4), 457-464.
Trinidad, C. V., Tetlow, A. L., Bantis, L. E., & Godwin, A. K. (2020). Reducing Ovarian Cancer Mortality Through Early Detection: Approaches Using Circulating Biomarkers. Cancer Prev Res (Phila), 13(3), 241-252.
Vandooren, J., & Itoh, Y. (2021). Alpha-2-Macroglobulin in Inflammation, Immunity and Infections. Front Immunol, 12, 803244.
Webster, J. A., Wuethrich, A., Shanmugasundaram, K. B., Richards, R. S., Zelek, W. M., Shah, A. K., Hill, M. M. (2021). Development of EndoScreen Chip, a Microfluidic Pre-Endoscopy Triage Test for Esophageal Adenocarcinoma. Cancers (Basel), 13(12).
Weeks, K. S., Herbach, E., McDonald, M., Charlton, M., & Schweizer, M. L. (2020). Meta-Analysis of VTE Risk: Ovarian Cancer Patients by Stage, Histology, Cytoreduction, and Ascites at Diagnosis. Obstet Gynecol Int, 2020, 2374716.
Wu, J., Xie, X., Liu, Y., He, J., Benitez, R., Buckanovich, R. J., & Lubman, D. M. (2012). Identification and confirmation of differentially expressed fucosylated glycoproteins in the serum of ovarian cancer patients using a lectin array and LC-MS/MS. J Proteome Res, 11(9), 4541-4552.
Wu, J., Xie, X., Nie, S., Buckanovich, R. J., & Lubman, D. M. (2013). Altered expression of sialylated glycoproteins in ovarian cancer sera using lectin-based ELISA assay and quantitative glycoproteomics analysis. J Proteome Res, 12(7), 3342-3352.
Zhang, W., Peng, P., Ou, X., Shen, K., & Wu, X. (2019). Ovarian cancer circulating extracelluar vesicles promote coagulation and have a potential in diagnosis: an iTRAQ based proteomic analysis. BMC Cancer, 19(1), 1095.

1. A method of:
(a) determining an indicator used in assessing a likelihood of the presence or absence of an ovarian cancer in a subject through screening or diagnosis, or
(b) determining an indicator used in monitoring therapy response or progression of an ovarian cancer present in a subject,
the method comprising, consisting, or consisting essentially of:
(i) determining a biomarker value that is measured or derived for a glycospecies of at least one glycoprotein biomarker in a sample obtained from the subject, wherein the at least one glycoprotein is selected from insulin-like growth factor-binding protein 3 (IBP3), complement component C9 (CO9), alpha-2-macroglobulin (A2MG), inter-alpha-trypsin inhibitor heavy chain 3 protein (ITIH3), alpha-1-antitrypsin (A1AT), haptoglobin (HPT), alpha-1-antichymotrypsin (AACT), alpha-1-acid glycoprotein 1 (A1AG1), alpha-1-acid glycoprotein 2 (A1AG2), alpha-2-HS-glycoprotein (FETUA), complement factor B (CFAB), fibrinogen alpha (FIBA), fibrinogen beta (FIBB), galectin-3-binding protein (LG3BP), gelsolin (GELS), inter-alpha-trypsin inhibitor heavy chain 2 protein (ITIH2), and inter-alpha-trypsin inhibitor heavy chain 4 protein (ITIH4),
(ii) determining the indicator using the biomarker value(s), wherein the indicator is at least partially indicative of the likelihood of the presence or absence of the ovarian cancer in the subject or the likelihood of the progression of the ovarian cancer in the subject;
optionally, wherein in (b), the indicator is determined by comparing a biomarker value in a first sample to a respective biomarker value in a second sample, wherein the second sample was taken at a later time than the first sample.

2-3. (canceled)

4. The method of claim 1, wherein the one or more glycoprotein is selected from IBP3, CO9, A2MG, ITIH3, A1AT, HPT, and AACT.

5. A method of treating an ovarian cancer in a subject, the method comprising administering to the subject an anti-cancer treatment,
wherein the subject is classified as likely to have ovarian cancer based on an indicator derived from biomarker values measured or derived for a glycospecies of one or more glycoprotein biomarkers in a sample obtained from the subject,
wherein the one or more glycoprotein is selected from insulin-like growth factor-binding protein 3 (IBP3), complement component C9 (CO9), alpha-2-macroglobulin (A2MG), inter-alpha-trypsin inhibitor heavy chain 3 protein (ITIH3), alpha-1-antitrypsin (A1AT), haptoglobin (HPT), alpha-1-antichymotrypsin (AACT), alpha-1-acid glycoprotein 1 (A1AG1), alpha-1-acid glycoprotein 2 (A1AG2 alpha-2-HS-glycoprotein (FETUA), coagulation factor X (FA10), complement factor B (CFAB), fibrinogen alpha (FIBA), fibrinogen beta (FIBB), galectin-3-binding protein (LG3BP), gelsolin (GELS), inter-alpha-trypsin inhibitor heavy chain 2 protein (ITIH2), and inter-alpha-trypsin inhibitor heavy chain 4 protein (ITIH4), and
wherein the indicator is at least partially indicative of the likelihood of the presence of ovarian cancer in the subject,
preferably wherein the anti-cancer treatment is surgery, chemotherapy, radiotherapy, and/or immunotherapy,
optionally wherein the surgery comprises removing all or part of the ovaries.

6. The method of claim 5, wherein the one or more glycoprotein is selected from IBP3, CO9, A2MG, ITIH3, A1AT, HPT, and AACT.

7-10. (canceled)

11. The method of claim 1, wherein individual glycospecies that are differentially expressed between a sample from a subject with ovarian cancer and a sample from a healthy subject are selected from Table 1:
TABLE 1

| | Glycoprotein | |
|---|---|---|
| Lectin | Overexpressed | Underexpressed |
| AAL | A1AG1, A1AG2, A1AT, | A2MG, |
| | [[A1BG,]] AACT, [[CERU,]] | FETUA, GELS, |
| | CFAB, CO9, | IBP3, [[ITIH1,]] ITIH2, |
| | [[FA5,]] FA10, FIBA, FIBB, | |
| | HPT, ITIH3, | |
| | ITIH4 | |
| SNA | A1AG1, A1AT, AACT, | A1AG2, A2AP, A2MG, |
| | CFAB, CO9, FA10, FIBA, | ALS, AMBP, ANGT, APOB, |
| | FIBB, HPT, HPTR, IC1, | C1QC, CBG, CHLE, CO6, |
| | ITIH3 | CO7, CPN2, FA10, FA12, |
| | | FETUA, GELS, HEMO, |
| | | HEP2, IBP3, ITIH1, ITIH2, |
| | | KNG1, PLMN, PON1, |
| | | PGRP2, THRB, TRFE |
| STL | CO9, FIBA, FIBB, HPT, | A2MG, A2AP, ALS, AMBP, |
| | HPTR, ITIH3, LUM | ANGT, C1QC, CBG, CHLE, |
| | | CPN2, FA10, FA12, FETUA, |
| | | GELS, HEP2, HPTR, IBP3, |
| | | ITIH1, ITIH2, ITIH4, |
| | | LG3BP, KNG1, PGRP2, |
| | | PLMN, PON1, TRFE |

12. A method of determining an indicator used in differentiating between the presence of an ovarian cancer or a benign ovarian neoplasm in a subject, the method comprising, consisting, or consisting essentially of:
(i) determining a biomarker value that is measured or derived for a glycospecies of at least one glycoprotein biomarker in a sample obtained from the subject, wherein the at least one glycoprotein is selected from insulin-like growth factor-binding protein 3 (IBP3), complement component 9(CO9), alpha-2-macroglobulin (A2MG), inter-alpha-trypsin inhibitor heavy chain 3 protein (ITIH3), alpha-1-antitrypsin (A1AT), haptoglobin (HPT), alpha-1-antichymotrypsin (AACT), alpha-1-acid glycoprotein 1 (A1AG1), alpha-1-acid glycoprotein 2 (A1AG2), complement factor B (CFAB), alpha-2-HS-glycoprotein (FETUA), fibrinogen alpha (FIBA), fibrinogen beta (FIBB), gelsolin (GELS), inter-alpha-trypsin inhibitor heavy chain 2 protein (ITIH2), inter-alpha-trypsin inhibitor heavy chain 4 protein (ITIH4), and galectin-3 binding protein (LG3BP),
(ii) determining the indicator using the biomarker value(s), wherein the indicator is at least partially indicative of the presence or absence of ovarian cancer or a benign ovarian neoplasm in the subject.

13. The method of claim 12, wherein the one or more glycoprotein is selected from IBP3, CO9, A2MG, ITIH3, A1AT, HPT, and AACT.

14. (canceled)

15. The method of claim 1, wherein one or more or all of the following apply:
(i) the biomarker value is at least partially indicative of a concentration of the glycospecies of one or more glycoproteins in the sample obtained from the subject;
(ii) the biomarker value includes the abundance of the glycospecies of one or more glycoproteins;
(iii) the biomarker value comprises a ratio of the level of the glycospecies of a glycoprotein to the total level of the glycoprotein in the sample;
(iv) the level of the glycospecies of a glycoprotein is reduced relative to the level of the biomarker that correlates with a healthy subject, and the indicator is thereby determined to be at least partially indicative of the subject having ovarian cancer;
(v) the level of the glycospecies of a glycoprotein is about the same as the level of the biomarker that correlates with a healthy subject, and the indicator is determined to be at least partially indicative of the absence of ovarian cancer in the subject;
(vi) the indicator comprises a biomarker value for at least 2 glycoprotein biomarkers; or
(vii) a first biomarker value is measured or derived for a first glycospecies of a glycoprotein and a second biomarker value is measured or derived for a second glycospecies of the glycoprotein, wherein the first glycospecies of the glycoprotein is differently expressed between a sample from a subject with ovarian cancer and a sample from a healthy subject, and the second glycospecies of the glycoprotein is not so differentially expressed.

16-21. (canceled)

22. The method of claim 1, wherein the level of an individual glycospecies of a glycoprotein is determined by contacting the sample with a glycan-binding molecule specific for the glycospecies of the glycoprotein, under conditions that permit binding of the glycan-binding molecule to the glycospecies of the glycoprotein.

23. The method of claim 22, wherein the glycan-binding molecule is selected from the group consisting of a lectin, a glycospecific antibody, a glycospecific aptamer, a glycospecific peptide, and a glycospecific small molecule.

24. The method of claim 23, wherein the lectin is selected from Aleuria aurantia lectin (AAL), Erythrina cristagalli agglutinin (ECA), floribunda agglutinin (WFA), Solanum tuberosum lectin (STL), concanavalin A (ConA), Sambucus nigra agglutinin (SNA), and Phaseolus vulgaris Leucoagglutinin (L-PHA), preferably wherein the lectin is selected from AAL, SNA and STL.

25. (canceled)

26. The method of claim 12, wherein individual glycospecies that are differentially expressed between a sample from a subject with ovarian cancer and a sample from a healthy subject are selected from Table 2:
TABLE 2
Glycoprotein

| Lectin | Overexpressed | Underexpressed |
|---|---|---|
| AAL | A1AG1, A1AG2, A1AT, | A2MG, GELS, IBP3, ITIH2 |
| | [[A1BG,]] AACT, | |
| | CFAB, CO9, | |
| | [[FA5,]] FA10, FIBA, FIBB, | |
| | HPT, ITIH3, ITIH4, | |
| SNA | A1AT, AACT, CO9, FA10, | A1AG2, A2AP, A2MG, ALS, |
| | FIBA, FIBB, HPT, HPTR, ITIH3 | AMBP, ANGT, APOB, C1QC, |
| | | CBG, CHLE, CO6, CO7, CPN2, |
| | | FA10, FA12, FETUA, GELS, |
| | | IBP3, ITIH1, ITIH2, KNG1, |
| | | PLMN, PON1, PGRP2, THRB, |
| | | TRFE |
| STL | CO9, FIBB, HPT, HPTR | A2MG, A2AP, ALS, AMBP, |
| | | ANGT, CBG, CHLE, CPN2, |
| | | FA10, FETUA, GELS, HPTR, |
| | | IBP3, ITIH1, ITIH2, KNG1, |
| | | PLMN, PON1, TRFE |

27. The method of claim 1, comprising determining two or more biomarker values that are measured or derived from two or more glycospecies of the same glycoprotein.

28. The method of claim 1, comprising determining three or more biomarker values that are measured or derived from three or more glycospecies of the same glycoprotein.

29. The method of claim 1, wherein the ovarian cancer is high grade serous ovarian cancer.

30. The method of claim 1, wherein upon determining that a subject is likely to have an ovarian cancer, further comprising exposing the subject to a treatment regimen for treating the ovarian cancer.

31. The method of claim 30, wherein the treatment regimen comprises surgery, radiotherapy, chemotherapy, or an immunotherapy.

32. The method of claim 31, wherein the surgery removes all or part of the ovaries.

33. The method of claim 1, wherein the determination method is performed by a person who exposes the subject to the treatment regimen.

34. The method of claim 1, wherein the sample from the subject is provided to another person who performs the determination method and provides the results of the determination method to the person who exposes the subject to the treatment regimen.

35-43. (canceled)
